SEQUENCE LISTING (116 sequences)

SEQ ID NO: 1 - DNA, 652 bp, Homo sapiens
gaattgccaattccttgtttgtgcaaaatggatttcatgtcaatgaggagtttttgcaaatgatgaaaaaatattttaatgcagcagtaaatcatgtggacttcagtcaaaatgtagccgtggccaactacatcaataagtgggtggagaataacacaaacaatctggtgaaagatttggtatccccaagggattttgatgctgccacttatctggccctcattaatgctgtctatttcaaggggaactggaagtcgcagtttaggcctgaaaatactagaaccttttctttcactaaagatgatgaaagtgaagtccaaattccaatgatgtatcagcaaggagaattttattatggggaatttagtgatggctccaatgaagctggtggtatctaccaagtcctagaaataccatatgaaggagatgaaataagcatgatgctggtgctgtccagacaggaagttcctcttgctactctggagccattagtcaaagcacagctggttgaagaatgggcaaactctgtgaagaagcaaaaagtagaagtatacctgcccaggttcacagtggaacaggaaattgatttaaaagatgttttgaaggctcttggaataactgaaaattttcatcaaagatgcaaattttgaca

SEQ ID NO: 2 - DNA, 1450 bp, Homo sapiens
ctggatagaacagctcaagccttgccacttcgggcttctcactgcagctgggcttggacttcggagttttgccattgccagtgggacgtctgagactttctccttcaagtacttggcagatcactctcttagcagggtctgcgcttcgcagccgggatgaagctggtttccgtcgccctgatgtacctgggttcgctcgccttcctaggcgctgacaccgctcggttggatgtcgcgtcggagtttcgaaagaagtggaataagtgggctctgagtcgtgggaagagggaactgcggatgtccagcagctaccccaccgggctcgctgacgtgaaggccgggcctgcccagacccttattcggccccaggacatgaagggtgcctctcgaagccccgaagacagcagtccggatgccgcccgcatccgagtcaagcgctaccgccagagcatgaacaacttccagggcctccggagctttggctgccgcttcgggacgtgcacggtgcagaagctggcacaccagatctaccagttcacagataaggacaaggacaacgtcgcccccaggagcaagatcagcccccagggctacggccgccggcgccggcgctccctgcccgaggccggcccgggtcggactctggtgtcttctaagccacaagcacacggggctccagcccccccgagtggaagtgctccccactttctttaggatttaggcgcccatggtacaaggaatagtcgcgcaagcatcccgctggtgcctcccgggacgaaggacttcccgagcggtgtggggaccgggctctgacagccctgcggagaccctgagtccgggaggcaccgtccggcggcgagctctggctttgcaagggcccctccttctgggggcttcgcttccttagccttgctcaggtgcaagtgccccagggggcggggtgcagaagaatccgagtgtttgccaggcttaaggagaggagaaactgagaaatgaatgctgagacccccggagcaggggtctgagccacagccgtgctcgcccacaaactgatttctcacggcgtgtcaccccaccagggcgcaagcctcactattacttgaactttccaaaacctaaagaggaaaagtgcaatgcgtgttgtacatacagaggtaactatcaatatttaagtttgttgctgtcaagattttttttgtaacttcaaatatagagatatttttgtacgttatatattgtattaagggcattttaaaagcaattatattgtcctccccctattttaagacgtgaatgtctcagcgaggtgtaaagttgttcgccgcgtggaatgtgagtgtgtttgtgtgcatgaaagagaaagactgattacctcctgtgtggaagaaggaaacaccgagtctctgtataatctatttacataaaatgggtgatatgcgaacagcaaacc

SEQ ID NO: 3 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 4 - DNA, 809 bp, Homo sapiens
atgcctgtcttcatcttgaaagaaaagctccaggtcccttctccagccacccagccccaagatggtgatgctgctgctgctgctttccgcactggctggcctcttcggtgcggcagagggacaagcatttcatcttgggaagtgccccaatcctccggtgcaggagaattttgacgtgaataagtatctcggaagatggtacgaaattgagaagatcccaacaacctttgagaatggacgctgcatccaggccaactactcactaatggaaaacggaaagatcaaagtgttaaaccaggagttgagagctgatggaactgtgaatcaaatcgaaggtgaagccaccccagttaacctcacagagcctgccaagctggaagttaagttttcctggtttatgccatcggcaccgtactggatcctggccaccgactatgagaactatgccctcgtgtattcctgtacctgcatcatccaactttttcacgtggattttgcttggatcttggcaagaaaccctaatctccctccagaaacagtggactctctaaaaaatatcctgacttctaataacattgatgtcaagaaaatgacggtcacagaccaggtgaactgccccaagctctcgtaaccaggttctacagggaggctgcacccactccatgttacttctgcttcgctttcccctaccccacccccccccataaagacaaaccaatcaaccacgacaaaggaagttgacctaaacatgtaaccatgccctaccctgttaccttgctagctgcaaaataaacttgttgctgacctgc

SEQ ID NO: 5 - protein, 189 aa, Homo sapiens
MVMLLLLLSALAGLFGAAEGQAFHLGKCPNPPVQENFDVNKYLGRWYEIEKIPTTFENGRCIQANYSLMENGKIKVLNQELRADGTVNQIEGEATPVNLTEPAKLEVKFSWFMPSAPYWILATDYENYALVYSCTCIIQLFHVDFAWILARNPNLPPETVDSLKNILTSNNIDVKKMTVTDQVNCPKLS

SEQ ID NO: 6 - DNA, 2127 bp, Homo sapiens
gaaagatggatcactccagctcaaagagaacatgtgggaatgaaaggacaggctgggcccaaaggagaaaagggtgatgctggggaggagcttcctggccctcctgaaccttctgggcctgttggacccacggcaggagcagaagcagagggctctggcctaggctggggctcggacgtcggctctggctctggtgacctggtgggcagtgagcagctgctgagaggtcctccaggacccccagggccacctggcttacctgggattccaggaaaaccaggaactgatgttttcatgggaccccctggatctcctggagaggatggacctgctggtgaacctgggcccccgggccctgagggacagcctggagttgatggagccaccggccttcccgggatgaaaggggagaagggagcaagagggcctaatggctcagttggtgaaaagggtgaccctggcaacagaggcttacctggacccccggggaaaaagggacaagctggccctcctggggtcatgggacccccagggcctcctggaccccctgggcccccaggccctggatgcacaatgggacttggattcgaggataccgaaggctctggaagcacccagctattgaatgaacccaaactctccagaccaacggctgcaattggtctcaaaggagagaaaggagaccggggacccaagggagaaagggggatggatggagccagtattgtgggaccccctgggccgagagggccacctgggcacatcaaggtcttgtctaattccttgatcaatatcacccatggattcatgaatttctcggacattcctgagctggtggggcctccggggccggacgggttgcctgggctgccaggatttccaggtcctagaggaccaaaaggtgacactggtttacctggctttccaggactaaaaggagaacagggcgagaagggagagccgggtgccatcctgacagaggacattcctctggaaaggctgatggggaaaaagggtgaacctggaatgcatggagccccaggaccaatggggcccaaaggaccaccaggacataaaggagaatttggccttcccgggcgacctggtcgcccaggactgaatggcctcaagggtaccaaaggagatccaggggtcattatgcagggcccacctggcttacctggccctccaggcccccctgggccacctggagctgtgattaacatcaaaggagccattttcccaatacccgtccgaccacactgcaaaatgccagttgatactgctcatcctgggagtccagagctcatcacttttcacggtgttaaaggagagaaaggatcctggggtcttcctggctcaaagggagaaaaaggcgaccagggagcccagggaccaccaggtcctccacttgatctagcttacctgagacactttctgaacaacttgaagggggagaatggagacaaggggttcaaaggtgaaaaaggagaaaaaggagacattaatggcagcttccttatgtctgggcctccaggcctgcccggaaatccaggcccggctggccaaaaaggggagacagtcgttgggccccaaggacccccaggtgctcctggtctgcctgggccacctggctttggaagacctggtgatcctgggccaccggggcccccggggccaccaggacctccagctatcctgggagcagctgtggcccttccaggtccccctggccctccaggacagccagggcttcccggatccagaaacctggtcacagcattcagcaacatggatgacatgctgcagaaagcgcatttggttatagaaggaacattcatctacctgagggacagcactgagtttttcattcgtgttagagatggctggaaaaaattacagctgggagaactgatccccattcctgccgacagccctccaccccctgcgctttccagcaacccacatcagcttctgcctccaccaaaccctatttcaagtgccaattatgagaagcctgctctgcatttggctgctctgaacatgccattttctggggacattcgagctgattttcagtgcttcaagcaggccagagctgcaggactgttgtccacctaccgagca

SEQ ID NO: 7 - DNA, 2384 bp, Homo sapiens
cctgctcgttcctgacagaattaacggcacagccaacaagatgaacggagctttggatcactcagaccaaccagacccagatgccattaagatgtttgtcggacagatcccccggtcatggtcggaaaaggagctgaaagaactttttgagccttacggagccgtctaccagatcaacgtcctccgggaccggagtcagaaccctccgcagagtaaaggttgttgtttcgtaacattttatacaagaaaagctgcacttgaggcccagaatgcactgcacaatattaaaactttacctgggatgcatcatcccattcagatgaaacctgcagatagtgaaaagtccaacgctgtggaagacagaaaattgttcataggaatggtttcgaagaaatgtaatgagaacgacatcagggtgatgttctctccatttggccagatagaagaatgccggatcctccggggacctgatgggctgagtcgaggctgtgcgtttgtcacattttctacaagggcaatggcacagaatgcaatcaaagccatgcatcagtctcagaccatggagggctgctcttcacctatcgtggtgaagtttgctgacactcagaaggacaaagagcaaaggcgcctccagcagcagctcgctcagcagatgcagcagctcaacactgccacctgggggaacctgacagggctgggcggactgaccccacagtatctggcgctcctgcagcaggccacctcctccagcaacctgggtgcgttcagcggcattcaacaaatggcaggcatgaatgctttacagttgcagaacctggcgacgctggctgctgctgcagctgcggcccagacctcagccaccagcaccaatgcaaaccctctctctaccacgagcagcgccctgggagccctcacgagtcccgtggctgcttcaacccccaactccactgctggtgcagccatgaactccttgacctctctcgggactctgcaaggactggctggagccactgttggactgaataatattaatgcactagcagttgctcaaatgctctcaggtatggcggctctgaatggaggacttggcgccacaggcttgacgaatggcacggctggcaccatggacgccctcacccaggcctactcaggaattcaacagtacgcagccgccgcgctgcccactctgtacagccagagcctgctgcagcagcagagcgctgcaggcagccagaaggaaggtccagagggggcaaacctctttatttaccaccttccacaggaatttggagaccagcacattctgcagatgttcatgccttttggaaatgttatctctgctaaagtcttcattgacaaacagaccaatctgagcaagtgctttggttttgttagctacgacaatccagtctctgcacaagctgctatccaagctatgaatggctttcagatcggcatgaaacgcttgaaggtgcagctgaagcgttccaaaaacgacagcaaaccttactgatcctaaccccagaggctccctgctctcattttagctttcttaggacatcttcatgcccgttagttcatcgtttgcctagcatgtccctgtggcgtctcaaaaaaaagtttcatcgtcccgtcattgtttctgatgtctttctgacctcacatcatatttggttctcctactgacctttgatctagtttgacctttgaaatttgcatgtgacctcatctagctatgaattctgggaagtcaatgtgaaaaacattgctgcattcatgcaagactgaaatttattattagacaaattcattatagaaaaaacctgtggcaaaaacgtttctttcttattttttttcttttcctaaaacagacttgaaagtattatacagggattggcattcttcccggtcactggtaacaatagcaatatgtgtccagggacacagaatgttggtttctaacagactacttccaaaaacagtttgagaaaaaaactgtctgattttaagtctctagaggtctgtaatagtttttacatttttcaggcagtgtaaagttttttgataaggccattttaggtggctcactttctcattaagatatatatatagaaccactttttgtagattagtataagaaaaatatttaccctgttttggggcaaatgctacctatttgtgtcaccttttgctgaactcacagttagacaatccatggtttaatgcacatgaaattacctatattttatactgtttcaatgtacaggagaaaggttactgtaaactgtgttatgttggtgcttctgtgaattaagttgtggtttcatcatgagtcttaatgttctttgttgataagacaagtttagaattggtttacttaatac

SEQ ID NO: 8 - protein, 490 aa, Homo sapiens
MNGALDHSDQPDPDAIKMFVGQIPRSWSEKELKELFEPYGAVYQINVLRDRSQNPPQSKGCCFVTFYTRKAALEAQNALHNIKTLPGMHHPIQMKPADSEKSNAVEDRKLFIGMVSKKCNENDIRVMFSPFGQIEECRILRGPDGLSRGCAFVTFSTRAMAQNAIKAMHQSQTMEGCSSPIVVKFADTQKDKEQRRLQQQLAQQMQQLNTATWGNLTGLGGLTPQYLALLQQATSSSNLGAFSGIQQMAGMNALQLQNLATLAAAAAAAQTSATSTNANPLSTTSSALGALTSPVAASTPNSTAGAAMNSLTSLGTLQGLAGATVGLNNINALAVAQMLSGMAALNGGLGATGLTNGTAGTMDALTQAYSGIQQYAAAALPTLYSQSLLQQQSAAGSQKEGPEGANLFIYHLPQEFGDQHILQMFMPFGNVISAKVFIDKQTNLSKCFGFVSYDNPVSAQAAIQAMNGFQIGMKRLKVQLKRSKNDSKPY

SEQ ID NO: 9 - DNA, 419 bp, Homo sapiens
tttttttttttttttttcagcatagtgaacgttaatatttttatttcttttgtaatgaagagtacaaatggttgggagcaggacatcacaggaggaggaaagatagcgccatctctgcagaagaactcctgagccacacacagaaggaaagttgatccccagggcagcctttcccaccaaaaaaatcaggcccaatccaggagagtttgccagtagctccccagggttccagggtgtctgccagccttcctaggaatcgtgggcaggcttctaggtgccagtgactcaaactcctttttccacttcccagttcaacctggtcactctcatccccacaagttcccaatctgaatcccattctctgaccattctctgcttccttgtttttaatctcatttgagagtgatcctcacgggttc

SEQ ID NO: 10 - DNA, 762 bp, Homo sapiens
gccgggaggagcggcgcgcgctcggacctctcccgccctgctcgttcgctctccagcttgggatggccggctacctgcgggtcgtgcgctcgctctgcagagcctcaggctcgcggccggcctgggcgccggcggccctgacagcccccacctcgcaagagcagccgcggcgccactatgccgacaaaaggatcaaggtggcgaagccgtggtggagatggatggtgatgagatgacccgtattatctggcagttcatcaaggagaagctcatcctgccccacgtggacatccagctaaagtattttgacctcgggctcccaaaccgtgaccagactgatgaccaggtcaccattgactctgcactggccacccagaagtacagtgtggctgtcaagtgtgccaccatcacccctgatgaggcccgtgtggaagagttcaagctgaagaagatgtggaaaagtcccaatggaactatccggaacatcctgggggggactgtcttccgggagcccatcatctgcaaaaacatcccacgcctagtccctggctggaccaagcccatcaccattggcaggcacgcccatggcgaccagtacaaggccacagactttgtggcagaccgggccggcactttcaaaatggtcttcaccccaaaagatggcagtggtgtcaaggagtgggaagtgtacaacttccccgcaggcgcgcgtgggcatgggcatgtacmacaccgacgagtccatctcaggttttgcgcaca

SEQ ID NO: 11 - DNA, 689 bp, Homo sapiens
aactccatctttgaaaaacatttaataatgtaatgtgtttgtggtacagggtgagtacagatgcacaggaggccatagggtttaggcaaaggggagcacaaaagttgaagatgaggcgctgccatcaatgctgggacttcaggccaagggcaggaactgaggaagccacaagggaggacattttctgcagttgctgaaccagtagcaactaggtcctgagaaagccctctctcgtggaagaataacagccaggcgggaaagcttttcatcctgcaaagctggggaagaagattcttccttaaattgtcatctgcacttcagctcaggaatcctgttggctgaagtccagagtgtcctttctgattcctgaagtagatgaacagcccggccccaaggaagagcaggcccagcacaaagcccccgactccactcagcatcttgctctgtgcagattcagaccgtgcactccattccactgtgagagggctcgttacgcttgggtgctccacttggcaggtgtaaacctctccactccgaggaactgtttctagcatcaccagggtctggaatgtccagtctccattctggatcaggcgcgtggacaccaccccagccttctcttcctgcccgttccggaaccagctgacttcaatgctgcctggatagaaaccactcacagagcagaccag

SEQ ID NO: 12 - DNA, 1959 bp, Homo sapiens
gggagcacgcgggagtccggctgcccacccatggcagggtctaccagaagggccacgagctggtgttggccaatattgctgaaagtgatgctggtgtctacacctgccacgcggccaacctggctggtcagcggagacaggatgtcaacatcactgtggccactgtgccctcctggctgaagaagccccaagacagccagctggaggagggcaaacccggctacttggattgcctgacccaggccacaccaaaacctacagttgtctggtacagaaaccagatgctcatctcagaggactcacggttcgaggtgttcctggcaaaggctcagggcttggaggagggagtggcagagaccctggtacttgtgaagagcctgcagagcaaggatgagcagcagcagctggacttccggagggagttggagatgtttgggaagctgaaccacgccaacgtggtgcggctcctggggctgtgccgggaggctgagccccactacatggtgctggaatatgtggatctgggagacctcaagcagttcctgaggatttccaagagcaaggatgaaaaattgaagtcacagcccctcagcaccaagcagaaggtggccctatgcacccaggtagccctgggcatggagcacctgtccaacaaccgctttgtgcataaggacttggctgcgcgtaactgcctggtcagtgcccagagacaagtgaaggtgtctgccctgggcctcagcaaggatgtgtacaacagtgagtactaccacttccgccaggcctgggtgccgctgcgctggatgtcccccgaggccatcctggagggtgacttctctaccaagtctgatgtctgggccttcggtgtgctgatgtgggaagtgtttacacatggagagatgccccatggtgggcaggcagatgatgaagtactggcagatttgcaggctgggaaggctagacttcctcagcccgagggctgcccttccaaactctatcggctgatgcagcgctgctgggccctcagccccaaggaccggccctccttcagtgagattgccagcgccctgggagacagcaccgtggacagcaagccgtgaggagggagcccgctcaggatggcctgggcaggggaggacatctctagagggaagctcacagcatgatgggcaagatccctgtcctcctgggccctgaggcccctgccctagtgcaacaggcattgctgaggtctgagcagggcctggcctttcctcctcttcctcaccctcatcctttgggaggctgacttggacccaaactgggcgactagggctttgagctgggcagttttccctgccacctcttcctctatcagggacagtgtgggtgccacaggtaaccccaatttctggccttcaacttctccccttgaccgggtccaactctgccactcatctgccaactttgcctggggagggctaggcttgggatgagctgggtttgtggggagttccttaatattctcaagttctgggcacacagggttaatgagtctcttggcccactggtcccacttgggggtctagaccaggattatagaggacacagcaagtgagtcctccccactctgggcttgtgcacactgacccagacccacgtcttccccacccttctctcctttcctcatcctaagtgcctggcagatgaaggagttttcaggagcttttgacactatataaaccgccctttttgtatgcaccacgggcggcttttatatgtaattgcagcgtggggtgggtgggcatgggaggtaggggtgggccctggagatgaggagggtgggccatccttaccccacacttttattgttgtcgttttttgtttgttttgtttttttgtttttgtttttgtttttacactcgctgctctcaataaataagccttttttacaaaaaaaaaaaaaaaaaaaaaaaaaaaaaa

SEQ ID NO: 13 - protein, 364 aa, Homo sapiens
EHAGVRLPTHGRVYQKGHELVLANIAESDAGVYTCHAANLAGQRRQDVNITVATVPSWLKKPQDSQLEEGKPGYLDCLTQATPKPTVVWYRNQMLISEDSRFEVFLAKAQGLEEGVAETLVLVKSLQSKDEQQQLDFRRELEMFGKLNHANVVRLLGLCREAEPHYMVLEYVDLGDLKQFLRISKSKDEKLKSQPLSTKQKVALCTQVALGMEHLSNNRFVHKDLAARNCLVSAQRQVKVSALGLSKDVYNSEYYHFRQAWVPLRWMSPEAILEGDFSTKSDVWAFGVLMWEVFTHGEMPHGGQADDEVLADLQAGKARLPQPEGCPSKLYRLMQRCWALSPKDRPSFSEIASALGDSTVDSKP

SEQ ID NO: 14 - DNA, 430 bp, Homo sapiens
ggttgatgtttcaacaaagtagttttaagttccatccattcagattttaagcattttgatttatttaaaaagggattgttcatagaaaaaaattactttgaacagtatacaggactggtggagattggctatttcacaacatcagacagtacaatcagtatctgccatatggctggtctctgtagacgccctttgctgtcctcgctgaaggtgccttgtgtcttgagttagtccactcttcttgcccgtaggaatcataagtctcctcactgagtccatgtccgtaatcatagtaatcagcaccactttgggctggggtgctatagctgttatcataggaatcataactctgttcatcataagcagtgccatatccatcatcatagtcatattctccataagtctcttggtgtcgggggtggcggtggaa

SEQ ID NO: 15 - DNA, 616 bp, Homo sapiens
ggtatgatgaccaaacataaaaagtgttttataattgttggtgttttaataacaactaatattattactctgatagttaaactaactcgagattctcagagtttatgcccctatgattggattggtttccaaaacaaatgctattatttctctaaagaagaaggagattggaattcaagtaaatacaactgttccactcaacatgccgacctaactataattgacaacatagaagaaatgaattttcttaggcggtataaatgcagttctgatcactggattggactgaagatggcaaaaaatcgaacaggacaatgggtagatggagctacatttaccaaatcgtttggcatgagagggagtgaaggatgtgcctacctcagcgatgatggtgcagcaacagctagatgttacaccgaaagaaaatggatttgcaggaaaagaatacactaagttaatgtctaagataatggggaaaatagaaaataacattattaagtgtaaaaccagcaaagtacttttttaattaaacaaagttcgagttttgtacctgtctggttaattctgcttacgtgtcaggctacacataaaagccacttcaaagattggcaaaaaa

SEQ ID NO: 16 - protein, 149 aa, Homo sapiens
MMTKHKKCFIIVGVLITTNIITLIVKLTRDSQSLCPYDWIGFQNKCYYFSKEEGDWNSSKYNCSTQHADLTIIDNIEEMNFLRRYKCSSDHWIGLKMAKNRTGQWVDGATFTKSFGMRGSEGCAYLSDDGAATARCYTERKWICRKRIH

SEQ ID NO: 17 - DNA, 1973 bp, Homo sapiens
gggatattggagtagcaagaggctgggaagccatcacttaccttgcactgagaaagaagacaaaggccagtatgcacagctttcctccactgctgctgctgctgttctggggtgtggtgtctcacagcttcccagcgactctagaaacacaagagcaagatgtggacttagtccagaaatacctggaaaaatactacaacctgaagaatgatgggaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgaggggaaccctcgctgggagcaaacacatctgacctacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcggctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaggttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaattgaacattactaatttgaatggaaaacacatggtgtgagtccaaagaaggtgttttcctgaagaactgtctattttctcagtcatttttaacctctagagtcactgatacacagaatataatcttatttatacctcagtttgcatatttttttactatttagaatgtagccctttttgtactgatataatttagttccacaaatggtgggtacaaaaagtcaagtttgtggcttatggattcatataggccagagttgcaaagatcttttccagagtatgcaactctgacgttgatcccagagagcagcttcagtgacaaacatatcctttcaagacagaaagagacaggagacatgagtctttgccggaggaaaagcagctcaagaacacatgtgcagtcactggtgtcaccctggataggcaagggataactcttctaacacaaaataagtgttttatgtttggaataaagtcaaccttgtttctactgtttt

SEQ ID NO: 18 - protein, 469 aa, Homo sapiens
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 19 - DNA, 1141 bp, Homo sapiens
ccgccaggcgctttctcggacgccttgcccagcgggccgcccgaccccctgcaccatggaccccgctcgccccctggggctgtcgattctgctgcttttcctgacggaggctgcactgggcgatgctgctcaggagccaacaggaaataacgcggagatctgtctcctgcccctagactacggaccctgccgggccctacttctccgttactactacgacaggtacacgcagagctgccgccagttcctgtacgggggctgcgagggcaacgccaacaatttctacacctgggaggcttgcgacgatgcttgctggaggatagaaaaagttcccaaagtttgccggctgcaagtgagtgtggacgaccagtgtgaggggtccacagaaaagtatttctttaatctaagttccatgacatgtgaaaaattcttttccggtgggtgtcaccggaaccggattgagaacaggtttccagatgaagctacttgtatgggcttctgcgcaccaaagaaaattccatcattttgctacagtccaaaagatgagggactgtgctctgccaatgtgactcgctattattttaatccaagatacagaacctgtgatgctttcacctatactggctgtggagggaatgacaataactttgttagcagggaggattgcaaacgtgcatgtgcaaaagctttgaaaaagaaaaagaagatgccaaagcttcgctttgccagtagaatccggaaaattcggaagaagcaattttaaacattcttaatatgtcatcttgtttgtctttatggcttatttgcctttatggttgtatctgaagaataatatgacagcatgaggaaacaaatcattggtgatttattcaccagtttttattaatacaagtcactttttaaaaaatttggatttttttatatataactagctgctattcaaatgtgagtctaccatttttaatttatggttcaactgtttgtgagactgaattcttgcaatgcataagatataaaagcaaatatgactcactcatttcttggggtcgtattcctgatttcagaagaggatcataactgaaacaacataagacaatataatcatgtgcttttaacatatttgagaataaaaaggactagc

SEQ ID NO: 20 - DNA, 22 bp, Artificial
  Sequenceserine (or cysteine) proteinase inhibitor sense primer
ccaaactttgaaagccaaggtg

SEQ ID NO: 21 - DNA, 21 bp, Artificial
  serine (or cysteine) proteinase inhibitor antisense primer
tgcacttcaaagaccagcagg

SEQ ID NO: 22 - DNA, 21 bp, Artificial
  adrenomedullin sense primer
aggaatagtcgcgcaagcatc

SEQ ID NO: 23 - DNA, 22 bp, Artificial
  adrenomedullin antisense primer
cacgcattgcacttttcctctt

SEQ ID NO: 24 - DNA, 21 bp, Artificial
  apolipoprotein D sense primer
tgaagccaccccagttaacct

SEQ ID NO: 25 - DNA, 23 bp, Artificial
  apolipoprotein D antisense primer
ggtcaacttcctttgtgcgtggt

SEQ ID NO: 26 - DNA, 22 bp, Artificial
  collagen type 15 alpha 1 sense primer
accaaaccctatttcaagtgcc

SEQ ID NO: 27 - DNA, 21 bp, Artificial
  collagen type 15 alpha 1 antisense primer
tagttatccacaaggcggacg

SEQ ID NO: 28 - DNA, 21 bp, Artificial
  CUG triplet  repeat  RNA-binding protein 2 sense primer
atgtttgtcggacagatcccc

SEQ ID NO: 29 - DNA, 21 bp, Artificial
  CUG triplet  repeat  RNA-binding protein 2 antisense primer
atgtgacaaacgcacagcctc

SEQ ID NO: 30 - DNA, 20 bp, Artificial
  dermatopontin sense primer
atttgaaccggcaaggcttc

SEQ ID NO: 31 - DNA, 21 bp, Artificial
  dermatopontinanti sense primer
aagtggttgttgctcctcgga

SEQ ID NO: 32 - DNA, 20 bp, Artificial
  isocitrate dehydrogenase sense primer
tgccatccagaagaaatggc

SEQ ID NO: 33 - DNA, 19 bp, Artificial
  isocitrate dehydrogenase antisense primer
aggcaaagatgctggcgat

SEQ ID NO: 34 - DNA, 22 bp, Artificial
  MHC Class 2 DR beta3 sense primer
tagctgtggacaaagccaacct

SEQ ID NO: 35 - DNA, 22 bp, Artificial
  MHC Class 2 DR beta3 antisense primer
ggcacacaccacgttctctgta

SEQ ID NO: 36 - DNA, 22 bp, Artificial
  protein tyrosine kinase 7 sense primer
tggaaccagagcgtacgactgt

SEQ ID NO: 37 - DNA, 19 bp, Artificial
  protein tyrosine kinase 7 antisense primer
tggctttgcagcgcttctt

SEQ ID NO: 38 - DNA, 22 bp, Artificial
  Sam-68 like phosphotyrosine protein, T-STAR sense primer
aggagaaggcaaggatgaagaa

SEQ ID NO: 39 - DNA, 22 bp, Artificial
  Sam-68 like phosphotyrosine protein, T-STAR antisense primer
tgttaactcctggagctgtgct

SEQ ID NO: 40 - DNA, 22 bp, Artificial
  C-type lectin superfamily member 2 sense primer
gcccctatgattggattggttt

SEQ ID NO: 41 - DNA, 22 bp, Artificial
  C-type lectin superfamily member 2 antisense primer
catccttcactccctctcatgc

SEQ ID NO: 42 - DNA, 25 bp, Artificial
  MMP (martix metalloproteinase) 1 sense primer
cgactctagaaacacaagagcaaga

SEQ ID NO: 43 - DNA, 25 bp, Artificial
  MMP (martix metalloproteinase) 1 antisense primer
aaggttagcttactgtcacacgctt

SEQ ID NO: 44 - DNA, 17 bp, Artificial
  TFPI (tissue factor pathway inhibitor) 2 sense primer
gtcgattctgcttttcc

SEQ ID NO: 45 - DNA, 20 bp, Artificial
  TFPI (tissue factor pathway inhibitor) 2 antisense primer
atggaattttctttggtgcg

SEQ ID NO: 46 - DNA, 1045 bp, Homo sapiens
ctcccgagctctactgactcccaacagagcgcccaagcaagaaaatggccataagtggagtccctgtgctaggatttttcatcatagctgtgctgatgagcgctcaggaatcatgggctatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaatgtacctccagaggtaactgtgctcacgaacagccctgtggaactgagagagcccaacgtcctcatctgtttcatcgacaagttcaccccaccagtggtcaatgtcacgtggcttcgaaatggaaaacctgtcaccacaggagtgtcagagacagtcttcctgcccagggaagaccaccttttccgcaagttccactatctccccttcctgccctcaactgaggacgtttacgactgcagggtggagcactggggcttggactgagcctcttctcaagcactgggaggttgatgctccaagcccgtctccagagacttacgagacgtggtgtgtgccctggggctgactgtgggtctggtcgggcttcctttgggaccctctttcatcatccacggcgagtgcgccaaaagcattgcccagaaacccagggggccctctgtaaggcctgtgcgggggcagaacggtcccttaaaaagaagaacctcgtgggcaaaacctggcgcaagacgtcatacaaagggggggcatataacacgcgtgccccagaacgaccgcactcagggctcgccgcctataacaccccggtgtggtacccccaaggcgcgctaaaactaaacagcgcgcacatagtagctcaggcgatacaccactcacatatataatagcaccgctaa

SEQ ID NO: 47 - DNA, 18 bp, Artificial
  adrenomedullin primer for RT-PCR
ggtttccgtcgccctgat

SEQ ID NO: 48 - DNA, 24 bp, Artificial
  adrenomedullin primer for RT-PCR
gagcccacttattccacttctttc

SEQ ID NO: 49 - DNA, 22 bp, Artificial
  matrix metaroproteinase 1 primer for RT-PCR
gatggacctggaggaaatcttg

SEQ ID NO: 50 - DNA, 24 bp, Artificial
  matrix metaroproteinase 1 primer for RT-PCR
ccgcaacacgatgtaagttgtact

SEQ ID NO: 51 - DNA, 21 bp, Artificial
  tissue factor pathway inhibitor 2 primer for RT-PCR
ggcaacgccaacaatttctac

SEQ ID NO: 52 - DNA, 26 bp, Artificial
  tissue factor pathway inhibitor 2 primer for RT-PCR
caaactttgggaactttttctatcct

SEQ ID NO: 53 - DNA, 23 bp, Artificial
  serine (or cysteine) proteinase inhibitor primer for PT-PCR
tgggtggagaataacacaaacaa

SEQ ID NO: 54 - DNA, 22 bp, Artificial
  serine (or cysteine) proteinase inhibitor primer for RT-PCR
ccagataagtggcagcatcaaa

SEQ ID NO: 55 - DNA, 28 bp, Artificial
  protein tyrosine kinase 7 primer for RT-PCR
gggagttccttaatattctcaagttctg

SEQ ID NO: 56 - DNA, 27 bp, Artificial
  protein tyrosine kinase 7
gctgtgtcctctataatcctggtctag

SEQ ID NO: 57 - DNA, 20 bp, Artificial
  collagen, type XV, alpha 1 primer for RT-PCR
ccagcaacccacatcagctt

SEQ ID NO: 58 - DNA, 23 bp, Artificial
  collagen, type XV, alpha 1 primer for RT-PCR
atgcagagcaggcttctcataat

SEQ ID NO: 59 - DNA, 29 bp, Artificial
  Sam68-like phosphotyrosine protein primer for RT-PCR
tcacaacatcagacagtacaatcagtatc

SEQ ID NO: 60 - DNA, 22 bp, Artificial
  Sam68-like phosphotyrosine protein primer for RT-PCR
acgggcaagaagagtggactaa

SEQ ID NO: 61 - DNA, 23 bp, Artificial
  apolipoprotein D primer for RT-PCR
tgagaagatcccaacaacctttg

SEQ ID NO: 62 - DNA, 24 bp, Artificial
  apolipoprotein D primer for PT-PCR
tgatctttccgttttccattagtg

SEQ ID NO: 63 - DNA, 22 bp, Artificial
  major histocompatibility complex  DR beta
ggctgaagtccagagtgtcctt

SEQ ID NO: 64 - DNA, 18 bp, Artificial
  major histocompatibility complex  DR beta primer for RT-PCR
gctgggcctgctcttcct

SEQ ID NO: 65 - DNA, 19 bp, Artificial
  major histocompatibility complex  DR alpha primer for RT-PCR
gcccagggaagaccacctt

SEQ ID NO: 66 - DNA, 23 bp, Artificial
  major histocompatibility complex  DR alpha primer for RT-PCR
cagtcgtaaacgtcctcagttga

SEQ ID NO: 67 - DNA, 27 bp, Artificial
  C-type lectin primer for RT-PCR
atccattttctttcggtgtaacatcta

SEQ ID NO: 68 - DNA, 23 bp, Artificial
  C-type lectin primer for RT-PCR
catgagagggagtgaaggatgtg

SEQ ID NO: 69 - DNA, 24 bp, Artificial
  CUG triplet repeat RNA-binding protein 2 primer for RT-PCR
catgaatgctttacagttgcagaa

SEQ ID NO: 70 - DNA, 19 bp, Artificial
  CUG triplet repeat RNA-binding protein 2 primer for RT-PCR
gcgctgctcgtggtagaga

SEQ ID NO: 71 - DNA, 22 bp, Artificial
  INTEGRIN, ALPHA 6 (ITGA6), MRNA
gatgacagtgttccccgatacc

SEQ ID NO: 72 - DNA, 20 bp, Artificial
  INTEGRIN, ALPHA 6 (ITGA6), MRNA
tgtaagtcagccacgccaaa

SEQ ID NO: 73 - DNA, 21 bp, Artificial
  SOLUTE CARRIER FAMILY 20 (PHOSPHATE TRANSPORTER), MEMBER 1
aatggagaagctgacatggcc

SEQ ID NO: 74 - DNA, 21 bp, Artificial
  SOLUTE CARRIER FAMILY 20 (PHOSPHATE TRANSPORTER), MEMBER 1
ccaaacccacagaccaacaca

SEQ ID NO: 75 - DNA, 21 bp, Artificial
  RIBONUCLEOTIDE REDUCTASE M2 POLYPEPTIDE (RRM2)
tccaaggacattcagcactgg

SEQ ID NO: 76 - DNA, 19 bp, Artificial
  RIBONUCLEOTIDE REDUCTASE M2 POLYPEPTIDE (RRM2), MRNA
atcgacgcaaaagaaccgg

SEQ ID NO: 77 - DNA, 22 bp, Artificial
  FOLLISTATIN (FST), TRANSCRIPT VARIANT FST317, MRNA
ggaagtccagtaccaaggcaga

SEQ ID NO: 78 - DNA, 19 bp, Artificial
  FOLLISTATIN (FST), TRANSCRIPT VARIANT FST317, MRNA
tggcattgtcactggcaca

SEQ ID NO: 79 - DNA, 20 bp, Artificial
  SPROUTY (DROSOPHILA) HOMOLOG 2 (SPRY2), MRNA
gccatccgaaacaccaatga

SEQ ID NO: 80 - DNA, 22 bp, Artificial
  SPROUTY (DROSOPHILA) HOMOLOG 2 (SPRY2), MRNA
tgccacagtcctcacacctgta

SEQ ID NO: 81 - DNA, 21 bp, Artificial
  RAB3B, MEMBER RAS ONCOGENE FAMILY (RAB3B), MRNA
atgctgatgacacgttcaccc

SEQ ID NO: 82 - DNA, 22 bp, Artificial
  RAB3B, MEMBER RAS ONCOGENE FAMILY (RAB3B), MRNA
ggcctgccttacactgatgttc

SEQ ID NO: 83 - DNA, 21 bp, Artificial
  SOLUTE CARRIER FAMILY 2 (FACILITATED GLUCOSE TRANSPORTER)
gccttttcgttaaccgctttg

SEQ ID NO: 84 - DNA, 19 bp, Artificial
  SOLUTE CARRIER FAMILY 2 (FACILITATED GLUCOSE TRANSPORTER)
tcctcgttgcggttgatga

SEQ ID NO: 85 - DNA, 21 bp, Artificial
  INTERLEUKIN 13 RECEPTOR, ALPHA 2 (IL13RA2), MRNA
tgaaggctatttgaagtcgcc

SEQ ID NO: 86 - DNA, 19 bp, Artificial
  INTERLEUKIN 13 RECEPTOR, ALPHA 2 (IL13RA2), MRNA
cttcgcttcaatgcccttg

SEQ ID NO: 87 - DNA, 21 bp, Artificial
  SERINE/THREONINE KINASE 12 (STK12), MRNA
tggaaacgtgtacttggctcg

SEQ ID NO: 88 - DNA, 21 bp, Artificial
  SERINE/THREONINE KINASE 12 (STK12), MRNA
accagccgaagtcagcaatct

SEQ ID NO: 89 - DNA, 22 bp, Artificial
  MINICHROMOSOME MAINTENANCE DEFICIENT (S. CEREVISIAE) 5
aagtttggcctgactaccagca

SEQ ID NO: 90 - DNA, 22 bp, Artificial
  MINICHROMOSOME MAINTENANCE DEFICIENT (S. CEREVISIAE)
ccagacgtgtataccccaatgg

SEQ ID NO: 91 - DNA, 22 bp, Artificial
  THYROID HORMONE RECEPTOR INTERACTOR 13 (TRIP13), MRNA
cctgtgtaaagcgttagcccag

SEQ ID NO: 92 - DNA, 22 bp, Artificial
  THYROID HORMONE RECEPTOR INTERACTOR 13 (TRIP13), MRNA
gtggcccaatgtactgcttgat

SEQ ID NO: 93 - DNA, 22 bp, Artificial
  KINESIN-LIKE 6 (MITOTIC CENTROMERE-ASSOCIATED KINESIN)
ccagctttaacgaagccatgac

SEQ ID NO: 94 - DNA, 22 bp, Artificial
  KINESIN-LIKE 6 (MITOTIC CENTROMERE-ASSOCIATED KINESIN)
gcatttaccagaacctgtccca

SEQ ID NO: 95 - DNA, 22 bp, Artificial
  CYCLIN-DEPENDENT KINASE INHIBITOR 3
tcggtttatgtgctcttccagg

SEQ ID NO: 96 - DNA, 22 bp, Artificial
  CYCLIN-DEPENDENT KINASE INHIBITOR 3
atcctcttaggtctcgcaggct

SEQ ID NO: 97 - DNA, 22 bp, Artificial
  CHROMOSOME CONDENSATION PROTEIN G (HCAP-G), MRNA
gctcgccagaaattcgagtcta

SEQ ID NO: 98 - DNA, 22 bp, Artificial
  CHROMOSOME CONDENSATION PROTEIN G (HCAP-G), MRNA
catgatcactttcagagtcggc

SEQ ID NO: 99 - DNA, 21 bp, Artificial
  CDC28 PROTEIN KINASE 1 (CKS1), MRNA
gcccactacccaagaaaccaa

SEQ ID NO: 100 - DNA, 22 bp, Artificial
  CDC28 PROTEIN KINASE 1 (CKS1), MRNA
cataaatccgcaagtcaccaca

SEQ ID NO: 101 - DNA, 20 bp, Artificial
  PROTEIN REGULATOR OF CYTOKINESIS 1 (PRC1), MRNA
cccaagtggaattgatgcga

SEQ ID NO: 102 - DNA, 22 bp, Artificial
  PROTEIN REGULATOR OF CYTOKINESIS 1 (PRC1), MRNA
cccctcacacactgcttcattt

SEQ ID NO: 103 - DNA, 21 bp, Artificial
  CELL DIVISION CYCLE 2, G1 TO S AND G2 TO M (CDC2), MRNA
ctgattttggccttgccagag

SEQ ID NO: 104 - DNA, 22 bp, Artificial
  CELL DIVISION CYCLE 2, G1 TO S AND G2 TO M (CDC2), MRNA
ggatgattcagtgccattttgc

SEQ ID NO: 105 - DNA, 21 bp, Artificial
  CDC20 (CELL DIVISION CYCLE 20, S. CEREVISIAE, HOMOLOG)
acagttcgcgttcgagagtga

SEQ ID NO: 106 - DNA, 21 bp, Artificial
  CDC20 (CELL DIVISION CYCLE 20, S. CEREVISIAE, HOMOLOG)
ttccactgagccgaaggatct

SEQ ID NO: 107 - DNA, 22 bp, Artificial
  LIKELY ORTHOLOG OF MATERNAL EMBRYONIC LEUCINE ZIPPER KINAS
tggctctctcccagtagcattc

SEQ ID NO: 108 - DNA, 21 bp, Artificial
  LIKELY ORTHOLOG OF MATERNAL EMBRYONIC LEUCINE ZIPPER KINAS
tcacttgcggtcacatcttcc

SEQ ID NO: 109 - DNA, 22 bp, Artificial
  MINICHROMOSOME MAINTENANCE DEFICIENT (S. CEREVISIAE) 7
agagaacacaaggattgcccag

SEQ ID NO: 110 - DNA, 22 bp, Artificial
  MINICHROMOSOME MAINTENANCE DEFICIENT (S. CEREVISIAE) 7
atgacaggagctgagacttggc

SEQ ID NO: 111 - DNA, 22 bp, Artificial
  CYCLIN A2 (CCNA2), MRNA
ggcactgctgctatgctgttag

SEQ ID NO: 112 - DNA, 21 bp, Artificial
  CYCLIN A2 (CCNA2), MRNA
tgaaggtccatgagacaaggc

SEQ ID NO: 113 - DNA, 19 bp, Artificial
  THYMIDINE KINASE 1, SOLUBLE (TK1), MRNA
ggcgcaatgagctgcatta

SEQ ID NO: 114 - DNA, 22 bp, Artificial
  THYMIDINE KINASE 1, SOLUBLE (TK1), MRNA
aaatggcttcctctggaaggtc

SEQ ID NO: 115 - DNA, 21 bp, Artificial
  cyclin-dependent kinase inhibitor 2A (melanoma, p16, inhibits CDK 4) (CDKN2A), mRNA
tccccgattgaaagaaccaga

SEQ ID NO: 116 - DNA, 22 bp, Artificial
  cyclin-dependent kinase inhibitor 2A (melanoma, p16, inhibits CDK 4) (CDKN2A), mRNA
aagagaagccagtaacccccct

(19) United States
(12) Patent Application Publication
(10) Pub. No.: US 2006/0166214 A1
(43) Pub. Date: Jul. 27, 2006

(54) Marker for detecting mesenchymal stem cell and method of distinguishing mesenchymal stem cell using the marker

(76) Inventors: Yukio Kato, Hiroshima (JP); Koichiro Tsuji, Hiroshima (JP); Chika Koike, Hiroshima (JP)

Correspondence Address: MORGAN & FINNEGAN, L.L.P., 3 WORLD FINANCIAL CENTER, NEW YORK, NY, 10281-2101, US

(21) Appl. No.: 10/548,681

(22) PCT Filed: Feb. 27, 2004
(86) PCT No.: PCT/JP04/02457

§ 371 Date: Jan. 17, 2006

(30) Foreign Application Priority Data
Mar. 10, 2003 (JP) 2003-63077

(51) Int. Cl. C12Q 1/68; C07H 21/04

(52) U.S. Cl. 435/6; 536/24.3

(57) ABSTRACT

The present invention provides a marker for detecting, separating/distinguishing a mesenchymal stem cell and a method for detecting, separating/distinguishing a mesenchymal stem cell by using the marker. A gene shown in the sequence listing is expressed specifically in a mesenchymal stem cell. The present invention comprises use of the gene as a marker for detecting mesenchymal stem cells. In addition, the present invention comprises a probe for detecting the mesenchymal stem cell marker gene and a PCR primer for amplifying the gene in a test cell in detecting the mesenchymal stem cell gene marker, and further comprises a polypeptide marker for detecting mesenchymal stem cells comprising a polypeptide wherein the mesenchymal stem cell marker gene of the present invention is expressed, an antibody for detecting the polypeptide marker which specifically binds to the polypeptide marker, and still further, a method for distinguishing and separating a mesenchymal stem cell by using a probe for detecting the mesenchymal stem cell marker gene, and an antibody which specifically binds to a polypeptide marker.

### TECHNICAL FIELD

The present invention relates to detection and separation/distinguishment of mesenchymal stem cells; particularly, a gene marker for detecting mesenchymal stem cells and a polypeptide marker for detecting mesenchymal stem cells used for detecting, separating/distinguishing mesenchymal stem cells; and a method for detecting and distinguishing mesenchymal stem cells by using the markers.

### BACKGROUND ART

Mesenchymal stem cells exist in the bone marrow, etc., of mammals, and are known as multipotent stem cells which differentiate into adipocytes, chondrocytes, and osteocytes. Due to their multipotency in differentiation, mesenchymal stem cells draw attention as xenograft, homograft and autograft materials for regenerative medicine of many tissues, such as bone, cartilage, tendon, muscle, fat, and periodontal tissue (Gene & Medicine, Vol. 4, No. 2 (2000) p 58-61). A general statement as to the present condition and prospect of study of mesenchymal stem cells has been issued recently, and there is a report concerning collection and culture of mesenchymal stem cells (Experimental Medicine, Vol. 19, No. 3 (February issue) 2001, p 350-356). Further, it has been reported that mesenchymal stem cells also exist in an adipose tissue (Tissue Engineering, P. A. Zuk et al., Multilineage cells from human adipose tissue: implications for cell-based therapies. 7: 211-228, 2001).

In recent years, some patent applications relating to culture, differentiation, etc., of mesenchymal stem cells have been published. For instance, Published Japanese Translation of PCT International Publication No. 11-506610 discloses a composition and method for maintaining the viability of human mesenchymal precursor cells in a serum-free environment; Published Japanese Translation of PCT International Publication No. 10-512756 discloses a method comprising contacting the mesenchymal stem cells with a bioactive factor, such as osteoinductive factor, adjunct factor for differentiation, chondroinductive factor which comprise prostaglandin, ascorbic acid, collagenous extracellular matrix, etc., in order to induce differentiation of mesenchymal stem cells; Japanese Laid-Open Patent Application No. 2000-217576 discloses a method for differentiating a multipotential mesenchymal stem cell into adipocytes, which includes incubation of the mulitpotential mesenchymal stem cell in the presence of prolactin or a substance with an equivalent effect, respectively.

The present inventors previously found that it is possible to proliferate mesenchymal stem cells remarkably rapidly while maintaining their differentiation capacity, by culturing mesenchymal stem cells in the presence of extracellular matrix of basement membrane, or in a medium containing a fibroblast growth factor (FGF), etc., and disclosed a culturing method capable of obtaining significantly larger amount of mesenchymal stem cells than a conventional culturing method (Japanese Laid-Open Patent Application No. 2003-52360). In addition, the present inventors disclosed a method for separating and collecting mesenchymal stem cells from oral tissues, in order to conduct separation and collection being safe for a collected parent body and easy for collecting, upon collecting mesenchymal stem cells (Japanese Laid-Open Patent Application No. 2003-52365).

Recently, with the development of regenerative medicine, mesenchymal stem cells draw attention as xenograft, homograft and autograft materials for regenerative medicine of many tissues, such as bone, cartilage, tendon, muscle, fat, periodontal tissue, because of their multipotency in differentiation. In order to use mesenchymal stem cells for regenerative medicine of tissues, it is necessary to collect the stem cells from body tissues first, to proliferate them, and to further differentiate and proliferate them to prepare a tissue. Mesenchymal stem cells exist in the bone marrow and periosteum, and it is necessary to develop a method for collecting mesenchymal stem cells from these tissues safely and easily for the practical use of these cells in tissue regenerative medicine. In addition, for the practical use of mesenchymal stem cells in tissue regenerative medicine, it is necessary to develop a technique for securing a sufficient amount of mesenchymal stem cells. In order to do this, it is important to develop a technique for culturing and proliferating the collected mesenchymal stem cells while maintaining their differentiation capacity.

As stated above, the present inventors have developed the method for conducting separation and collection being safe for a collected parent body and easy for collecting, upon collecting mesenchymal stem cells. Further, the present inventors have developed a method for explosively proliferating mesenchymal stem cells while maintaining their differentiation capacity by culturing mesenchymal stem cells in the presence of extracellular matrix of basement membrane, or in a medium containing a fibroblast growth factor (FGF), etc. However, for the purpose of practical use of the cultured and proliferated mesenchymal stem cells in regenerative medicine, it is necessary to confirm that the cultured cells are mesenchymal stem cells, and to develop a method for detecting and distinguishing the mesenchymal stem cells. As for mesenchymal stem cells, marker genes that characterize the cells have not been identified conventionally. Therefore, for the purpose of practical use of mesenchymal stem cells in regenerative medicine, the identification of marker genes that characterize mesenchymal stem cells, and the development of a method for detecting, and separating/distinguishing mesenchymal stem cells are important problems to be solved.

The present invention provides a gene marker for detecting mesenchymal stem cells and a polypeptide marker for detecting mesenchymal stem cells used for detecting and distinguishing mesenchymal stem cells; a primer for PCR amplification for detecting the gene marker; and a method for detecting and distinguishing mesenchymal stem cells by using the markers and the primer.

In order to find a gene which can serve as a marker for detecting mesenchymal stem cells, the present inventors compared the expression of various genes in mesenchymal stem cells, and searched for a gene which can serve as a marker. As a result, the present inventors found that there was a difference between fibroblasts and mesenchymal stem cells in the expression levels of 13 genes shown in the sequence listing, and the genes were expressed specifically in mesenchymal stem cells, and that the genes could serve as a marker for detecting mesenchymal stem cells. The present invention has been thus completed. The present invention comprises a probe for detecting mesenchymal stem cell marker genes, and a PCR primer for amplifying the genes in test cell upon detecting the mesenchymal stem cell gene markers Further, the present invention comprises a polypeptide marker for detecting mesenchymal stem cells comprising a polypeptide wherein the mesenchymal stem cell marker gene of the present invention is expressed, and an antibody for detecting the polypeptide marker, which specifically binds to the polypeptide marker. Still further, the present invention comprises a method for distinguishing and separating mesenchymal stem cells using the probe for detecting mesenchymal stem cell marker genes, and the antibody which specifically binds to the polypeptide marker.

The process of the completion of the present invention is as follows: among various stem cells, mesenchymal stem cells are capable of differentiating into many tissues such as bone, cartilage, tendon, fat, skeletal muscle, cardiac muscle, blood vessel, nerve, etc., and are significantly expected as cells for regenerative medicine. However, marker genes that characterize mesenchymal stem cells have not been identified. Therefore, the present inventors have conducted a keen search for identifying marker genes that characterize mesenchymal stem cells. In brief, the search comprises the following steps: proliferating mesenchymal stem cells with the method, previously developed by the present inventors, wherein mesenchymal stem cells derived from bone marrow are cultured in the presence of FGF, etc., thereby explosively proliferating the cells while maintaining their differentiation capacity; culturing the mesenchymal stem cells obtained by this method and human fibroblasts in the presence of FGF; extracting mRNA from the cultured cells; by using the mRNA as a template and a DNA chip (Incyte) as a probe, comparing the genes expressed in human-derived mesenchymal stem cells and fibroblasts by DNA microarray technology.

Among 9400 genes, 63 genes exhibited significantly high expression, and 141 genes exhibited significantly low expression in mesenchymal stem cells. Among them, 87 genes (29 genes exhibiting high expression and 58 genes exhibiting low expression in mesenchymal stem cells) exhibited a difference, of three-fold or more, in expression levels. Total RNAs were extracted from mesenchymal stem cells derived from 3 individuals and fibroblasts derived from 3 individuals, and by using them as a template, the expression levels of the above-mentioned 87 genes were examined by semiquantitative RT-PCR. As a result, most genes exhibited no difference in the expression levels. In addition, there were many cases wherein differences in the expression were considered to be caused by individual differences, because differences in the expression levels were observed between mesenchymal stem cells, or fibroblasts, from different individuals.

There were 3 genes (tissue factor pathway inhibitor 2, major histocompatibility complex class2 DR beta 3, serine (or cysteine) proteinase inhibitor) whose high expression in mesenchymal stem cells was confirmed by RT-PCR, and 10 genes (matrixmetalloprotease 1, collagenase type XV alpha, CUG triplet repeat RNA binding protein, dermatopontin, protein tyrosine kinase 7, isocitrate dehydrogenase 2, Sam68-like phosphotyrosine protein, C-type lectin superfamily member 2, adrenomedullin, apolopoprotein D) whose low expression in mesenchymal stem cells was confirmed by RT-PCR. By measuring the expression levels of these 13 genes, it has become possible to identify mesenchymal stem cells, which are difficult to be determined, in a short time and at a low cost. In addition, as a result of gene search in the present invention, many genes exhibiting differences in the expression levels between mesenchymal stem cells, or fibroblasts, have been confirmed. These genes serve as a gene marker for detecting mesenchymal stem cells, and can be used for detecting mesenchymal stem cells as microarray, DNA chip or the like by constructing a probe for detecting genes.

### DISCLOSURE OF THE INVENTION

The present invention specifically comprises: a gene marker for detecting a mesenchymal stem cell which is a gene having a base sequence shown in SEQ ID NO: 1, 2, 4, 6, 7, 9, 10, 11, 12, 14, 15, 17, 19 or 46 in the sequence listing (“1”), a gene marker for detecting a mesenchymal stem cell which is a gene of INTEGRIN, ALPHA 6 (ITGA6), MRNA (NM_{—}000210); a gene of SOLUTE CARRIER FAMILY 20 (PHOSPHATE TRANSPORTER), MEMBER 1 (NM_{—}005415); a gene of RIBONUCLEOTIDE REDUCTASE M2 POLYPEPTIDE (RRM2), MRNA (NM_{—}001034); a gene of FOLLISTATIN (FST), TRANSCRIPT VARIANT FST317, MRNA (NM_{—}006350); a gene of SPROUTY (DROSOPHILA) HOMOLOG 2 (SPRY2), MRNA (NM_{—}005842); a gene of RAB3B, MEMBER RAS ONCOGENE FAMILY (RAB3B), MRNA (NM_{—}002867); a gene of SOLUTE CARRIER FAMILY 2 (FACILITATED GLUCOSE TRANSPORTER) (NM_{—}006516); a gene of INTERLEUKIN 13 RECEPTOR, ALPHA 2 (IL13RA2), MRNA (NM_{—}000640); a gene of SERINE/THREONINE KINASE 12 (STK12), MRNA (NM_{—}004217); a gene of MINICHROMOSOME MAINTENANCE DEFICIENT (S. CEREVISIAE) 5 (CE) (NM_{—}006739); a gene of THYROID HORMONE RECEPTOR INTERACTOR 13 (TRIP13), MRNA (NM_{—}004237); a gene of KINESIN-LIKE 6 (MITOTIC CENTROMERE-ASSOCIATED KINESIN) (K) (NM_{—}006845); a gene of CYCLIN-DEPENDENT KINASE INHIBITOR 3 (CDK2-ASSOCIATED DUAL (NM_{—}005192); a gene of CHROMOSOME CONDENSATION PROTEIN G (HCAP-G), MRNA (NM_{—}022346); a gene of CDC28 PROTEIN KINASE 1 (CKS1), MRNA (NM_{—}001826); a gene of PROTEIN REGULATOR OF CYTOKINESIS 1 (PRC1), MRNA (NM_{—}003981); a gene of CELL DIVISION CYCLE 2, G1 TO S AND G2 TO M (CDC2), MRNA (NM_{—}001786); a gene of CDC20 (CELL DIVISION CYCLE 20, S. CEREVISIAE, HOMOLOG) (CD) (NM_{—}001255); a gene of LIKELY ORTHOLOG OF MATERNAL EMBRYONIC LEUCINE ZIPPER KINAS (NM_{—}014791); a gene of MINICHROMOSOME MAINTENANCE DEFICIENT (S. CEREVISIAE) 7 (M) (NM_{—}005916); a gene of CYCLIN A2 (CCNA2), MRNA (NM_{—}001237); a gene of THYMIDINE KINASE 1, SOLUBLE (TK1), MRNA (NM_{—}003258); or a gene of cyclin-dependent kinase inhibitor 2A (melanoma, p16, inhibits CDK4) (CDKN2A), mRNA (NM_{—}000077) (“2”), a gene marker for detecting a mesenchymal stem cell which is a gene of EGF-containing fibulin-like extracellular matrix protein 1 (NM_{—}018894); a gene of Human insulin-like growth factor binding protein 5 (IGFBP5) mRNA (AU132011); a gene of Homo sapiens clone 24775 mRNA sequence (AA402981); proteoglycan 1, secretory granule (AV734015); a gene of insulin-like growth factor binding protein 5 (AA374325); a gene of solute carrier family 21 (organic anion transporter), member 3 (AF085224); a gene of transglutaminase 2 (C polypeptide, protein-glutamine-gamma-glutamyl transferase) (AL552373); a gene of coagulation factor II (thrombin) receptor (M62424); a gene of plasminogen activator, urokinase (NM_{—}002658); a gene of tissue inhibitor of metalloproteinase 3 (Sorsby fundus dystrophy, pseudoinflammatory) (W96324); a gene of matrix Gla protein (BF668572); a gene of B-cell CLL/lymphoma 1 (Z23022); a gene of CD74 antigen (invariant polypeptide of major histocompatibility complex, class II antigen-associated) (BG333618); a gene of adducin 3 (gamma) (AL135243); a gene of solute carrier family 16 (monocarboxylic acid transporters), member 4 (NM_{—}004696); a gene of protein S (alpha) (NM_{—}000313); a gene of phosphatidylinositol (4,5) bisphosphate 5-phosphatase homolog; phosphatidylinositol polyphosphate 5-phosphatase type IV (AF187891); a gene of progesterone membrane binding protein (BE858855); a gene of brain-derived neurotrophic factor (X60201); or a gene of apolipoprotein E (BF967316) (“3”), a gene marker for detecting a mesenchymal stem cell which is a gene of HOMO SAPIENS WNT INHIBITORY FACTOR-1 (WIF-1), MRNA(NM_{—}007191); a gene of HOMO SAPIENS HYPOTHETICAL PROTEIN FLJ11175 (FLJ11175), MRNA (NM_{—}018349); a gene of HOMO SAPIENS RHO GDP DISSOCIATION INHIBITOR (GDI) BETA (ARHGDIB), MRNA (NM_{—}001175); a gene of HOMO SAPIENS POTASSIUM INTERMEDIATE/SMALL CONDUCTANCE CALCIUM-ACTIVATE (NM_{—}021614); a gene of HOMO SAPIENS NUCLEAR FACTOR (ERYTHROID-DERIVED 2)-LIKE 3 (NFE2L3), MRNA (NM_{—}004289); a gene of HOMO SAPIENS GS3955 PROTEIN (GS3955), MRNA (NM_{—}021643); a gene of HOMO SAPIENS CDNA 3′ END SIMILAR TO SIMILAR TO GLYCOPROTEIN MUC18 (AA302605); a gene of HOMO SAPIENS HYPOTHETICAL PROTEIN FLJ21841 (FLJ21841), MRNA (NM_{—}024609); a gene of HOMO SAPIENS ZINC FINGER PROTEIN 185 (LIM DOMAIN) (ZNF185), MRNA (NM_{—}007150); a gene of SC1=PUTATIVE TRANS-ACTING FACTOR INVOLVED IN CELL CYCLE CONTROL [HUMAN, MRNA, 24] (S53374); a gene of HOMO SAPIENS NADH DEHYDROGENASE (UBIQUINONE) 1 ALPHA SUBCOMPLEX, 8 (19K) (NM_{—}014222); a gene of HOMO SAPIENS HOMOLOG OF YEAST MCM10; HYPOTHETICAL PROTEIN PRO2249 (PRO2) (NM_{—}018518); a gene of HOMO SAPIENS COLLAGEN, TYPE VII, ALPHA 1 (EPIDERMOLYSIS BULLOSA, DYSTRO (NM_{—}000094); a gene of HOMO SAPIENS AUTOCRINE MOTILITY FACTOR RECEPTOR (AMFR), MRNA (NM_{—}001144); a gene of HOMO SAPIENS CLONE 24421 MRNA SEQUENCE /CDS=UNKNOWN/GB=AF070641/GI=3283914/UG=(AF070641); a gene of HOMO SAPIENS MRNA; a gene of CDNA DKFZP586E1621 (FROM CLONE DKFZP586E1621)/CDS=UNKNOWN/GB (AL080235) (“4”), a gene marker for detecting a mesenchymal stem cell which is a gene of HOMO SAPIENS RESERVED (KCNK12), MRNA (NM_{—}022055); a gene of HOMO SAPIENS UBIQUITIN-CONJUGATING ENZYME E2C (UBE2C), MRNA (NM_{—}007019); a gene of HOMO SAPIENS CDNA FLJ10517 FIS, CLONE NT2RP2000812/CDS=(61,918)/GB=AK001379/G (AK001379); a gene of HOMO SAPIENS CARTILAGE LINKING PROTEIN 1 (CRTL1), MRNA (NM_{—}001884); a gene of HOMO SAPIENS MRNA; CDNA DKFZP434F2322 (FROM CLONE DKFZP434F2322)/CDS=UNKNOWN/GB (AL133105); a gene of HOMO SAPIENS, CLONE MGC:9549 IMAGE:3857382, MRNA, COMPLETE CDS/CDS=(92,1285)/G (BC012453); a gene of HOMO SAPIENS HYPOTHETICAL PROTEIN DKFZP586J1119 (DKFZP586J1119), MRNA (NM_{—}032270); a gene of HOMO SAPIENS GLIAMATURATION FACTOR, GAMMA (GMFG), MRNA (NM_{—}004877); a gene of HOMO SAPIENS CDNA: FLJ23095 FIS, CLONE LNG07413/CDS=UNKNOWN/GB=AK026748/GI=10 (AK026748); a gene of HOMO SAPIENS HYPOTHETICAL PROTEIN DKFZP762E1312 (DKFZP762E1312), MRNA (NM_{—}018410); a gene of HOMO SAPIENS IROQUOIS-CLASS HOMEODOMAIN PROTEIN (IRX-2A), MRNA (NM_{—}005853); or a gene of HOMO SAPIENS HYPOTHETICAL PROTEIN FLJ10540 (FLJ10540), MRNA (NM_{—}018131) (“5”), a gene marker for detecting a mesenchymal stem cell which is a gene of HOMO SAPIENS MRNA; CDNA DKFZP434C1915 (FROM CLONE DKFZP434C1915); PARTIAL CDS/C (AL137698); a gene of HOMO SAPIENS MINICHROMOSOME MAINTENANCE DEFICIENT (S. CEREVISIAE) 7 (M) (NM_{—}005916); a gene of HOMO SAPIENS DYNEIN, CYTOPLASMIC, INTERMEDIATE POLYPEPTIDE 1 (DNCI1), M (NM_{—}004411); a gene of HUMAN MRNA FOR PROTEIN GENE PRODUCT (PGP) 9.5. (X04741); a gene of HOMO SAPIENS RAD51-INTERACTING PROTEIN (PIR51), MRNA (NM_{—}006479); a gene of HOMO SAPIENS BACULOVIRAL IAP REPEAT-CONTAINING 5 (SURVIVIN) (BIRC5), MR (NM_{—}001168); a gene of HOMO SAPIENS UDP-N-ACETYL-ALPHA-D-GALACTOSAMINE:POLYPEPTIDE N-ACETYLGAL (NM_{—}004482); a gene of HOMO SAPIENS CYCLIN B1 (CCNB1), MRNA (NM_{—}031966); a gene of HOMO SAPIENS FLAP STRUCTURE-SPECIFIC ENDONUCLEASE 1 (FEN1), MRNA (NM_{—}004111); a gene of HOMO SAPIENS SERINE/THREONINE KINASE 15 (STK15), MRNA (NM_{—}003600); a gene of HOMO SAPIENS MAD2 (MITOTIC ARREST DEFICIENT, YEAST, HOMOLOG)-LIKE 1 (MA) (NM_{—}002358); a gene of HOMO SAPIENS NUCLEOLAR PROTEIN ANKT (ANKT), MRNA (NM_{—}018454); a gene of HOMO SAPIENS HSPC150 PROTEIN SIMILAR TO UBIQUITIN-CONJUGATING ENZYME (H) (NM_{—}014176); a gene of HOMO SAPIENS CYTOCHROME P450 RETINOID METABOLIZING PROTEIN (P450RAI-2), (NM_{—}019885); a gene of HOMO SAPIENS NIMA (NEVER IN MITOSIS GENE A)-RELATED KINASE 2 (NEK2), MR (NM_{—}002497); a gene of HOMO SAPIENS BONE MORPHOGENETIC PROTEIN 4 (BMP4), MRNA (NM_{—}001202); a gene of HOMO SAPIENS CDNA FLJ10674 FIS, CLONE NT2RP2006436/CDS=UNKNOWN/GB=AK001536/GI (AK001536); or a gene of HOMO SAPIENS CENTROMERE PROTEIN A (17 KD) (CENPA), MRNA (NM_{—}001809) (“6”), a gene marker for detecting a mesenchymal stem cell which is a gene of HOMO SAPIENS MRNA; CDNA DKFZP564P116 (FROM CLONE DKFZP564P116)/CDS=UNKNOWN/GB=A (AL049338); a gene of HOMO SAPIENS TTK PROTEIN KINASE (TTK), MRNA (NM_{—}003318); a gene of HOMO SAPIENS KARYOPHERIN ALPHA 2 (RAG COHORT 1, IMPORTIN ALPHA 1) (KPNA (NM_{—}002266); a gene of HOMO SAPIENS POLYMERASE (DNA DIRECTED), THETA (POLQ), MRNA (NM_{—}006596); a gene of HOMO SAPIENS ETS VARIANT GENE 5 (ETS-RELATED MOLECULE) (ETV5), MRNA (NM_{—}004454); a gene of HOMO SAPIENS TRANSFORMING, ACIDIC COILED-COIL CONTAINING PROTEIN 3 (TAC) (NM_{—}006342); a gene of HOMO SAPIENS KINESIN-LIKE 1 (KNSL1), MRNA (NM_{—}004523); a gene of HOMO SAPIENS CENTROMERE PROTEIN E (312 KD) (CENPE), MRNA (NM_{—}001813); a gene of HOMO SAPIENS CDNA, 3′ END/CLONE=IMAGE:1651289/CLONE_END=3′/GB=AI12 (AI123815); a gene of HOMO SAPIENS DISTAL-LESS HOMEO BOX5 (DLX5), MRNA (NM_{—}005221); a gene of HOMO SAPIENS V-MYB AVIAN MYELOBLASTOSIS VIRAL ONCOGENE HOMOLOG-LIKE 2 (NM_{—}002466); or a gene of HOMO SAPIENS SERUM DEPRIVATION RESPONSE (PHOSPHATIDYLSERINE-BINDING PRO (NM_{—}004657) (“7”), a gene marker for detecting a mesenchymal stem cell which is a gene of HOMO SAPIENS HYPOTHETICAL PROTEIN FLJ10604 (FLJ10604), MRNA (NM_{—}018154); a gene of HOMO SAPIENS RIBONUCLEASE HI, LARGE SUBUNIT (RNASEHI), MRNA (NM_{—}006397); a gene of HOMO SAPIENS ANILLIN (DROSOPHILA SCRAPS HOMOLOG), ACTIN BINDING PROTEIN (NM_{—}018685); a gene of HOMO SAPIENS H4 HISTONE FAMILY, MEMBER G (H4FG), MRNA (NM_{—}003542); a gene of HOMO SAPIENS G PROTEIN-COUPLED RECEPTOR 37 (ENDOTHELIN RECEPTOR TYPE B-(NM_{—}005302) HOMO SAPIENS UBIQUITIN CARRIER PROTEIN (E2-EPF), MRNA (NM_{—}014501); a gene of HOMO SAPIENS, SIMILAR TO TUMOR DIFFERENTIALLY EXPRESSED 1, CLONE IMAGE:3639252, (BC007375); a gene of HOMO SAPIENS HYPOTHETICAL PROTEIN MGC2577 (MGC2577), MRNA (NM_{—}031299); a gene of HOMO SAPIENS HYPOTHETICAL PROTEIN FLJ13912 (FLJ13912), MRNA (NM_{—}022770); a gene of HOMO SAPIENS ANNEXIN A3 (ANXA3), MRNA (NM_{—}005139); a gene of HOMO SAPIENS STATHMIN 1/ONCOPROTEIN 18 (STMN1), MRNA (NM_{—}005563) HOMO SAPIENS, SIMILAR TO RIBOSOMAL PROTEIN L39, CLONE MGC:20168 IMAGE:4555759, M (BC012328); or a gene of HOMO SAPIENS POLO (DROSOPHIA)-LIKE KINASE (PLK), MRNA (NM_{—}005030) (“8”), a gene marker for detecting a mesenchymal stem cell which is a gene of HOMO SAPIENS PROTEASOME (PROSOME, MACROPAIN) 26S SUBUNIT, ATPASE, 5 (PS) (NM_{—}002805); a gene of HOMO SAPIENS EUKARYOTIC TRANSLATION INITIATION FACTOR 4 GAMMA, 2 (EIF4G) (NM_{—}001418); a gene of HOMO SAPIENS DIHYDROPYRIMIDINASE-LIKE 3 (DPYSL3), MRNA) (NM 001387); a gene of HOMO SAPIENS ADAPTOR-RELATED PROTEIN COMPLEX 1, SIGMA 2 SUBUNIT (AP1S2) (NM_{—}003916); a gene of HOMO SAPIENS PLECTIN 1, INTERMEDIATE FILAMENT BINDING PROTEIN, 500 KD (P) (NM_{—}000445); a gene of HOMO SAPIENS HETEROGENEOUS NUCLEAR RIBONUCLEOPROTEIN U (SCAFFOLD ATTAC (NM_{—}031844); a gene of HOMO SAPIENS HYPOTHETICAL PROTEIN MGC5306 (MGC5306), MRNA (NM_{—}024116); a gene of HOMO SAPIENS MHC CLASS I POLYPEPTIDE-RELATED SEQUENCE A (MICA), MRNA (NM_{—}000247); a gene of HOMO SAPIENS PROTEIN ASSOCIATED WITH PRK1 (AWP1), MRNA (NM_{—}019006); a gene of HOMO SAPIENS PROGESTERONE RECEPTOR MEMBRANE COMPONENT 2 (PGRMC2), MRNA (NM_{—}006320); a gene of HOMO SAPIENS POLYGLUTAMINE BINDING PROTEIN 1 (PQBP1), MRNA (NM_{—}005710); a gene of HOMO SAPIENS S-ADENOSYLMETHIONINE DECARBOXYLASE 1 (AMD1), MRNA (NM_{—}001634); HOMO SAPIENS INTERFERON—INDUCED, HEPATITIS C-ASSOCIATED MICROTUBULAR AG (NML006417); a gene of HOMO SAPIENS PROTEIN KINASE, AMP-ACTIVATED, GAMMA 1 NON-CATALYTIC SUBUN (NM_{—}002733); HOMO SAPIENS PROCOLLAGEN-PROLINE, 2-OXOGLUTARATE 4-DIOXYGENASE (PROLINE (NM_{—}004199); a gene of HOMO SAPIENS ENDOTHELIAL DIFFERENTIATION, LYSOPHOSPHATIDIC ACID G-PROTE (NM_{—}012152); or a gene of HOMO SAPIENS KIAA0127 GENE PRODUCT (KIAA0127), MRNA (NM_{—}014755) (“9”), a probe for detecting a mesenchymal stem cell marker gene having a DNA sequence which hybridizes with the marker gene for detecting a mesenchymal stem cell according to any one of “1” to “9” under a stringent condition (“10”), the probe for detecting a mesenchymal stem cell marker gene according to “10”, which comprises whole or part of an antisense strand of the base sequence according to any one of “1” to “9” (“11”), a microarray or a DNA chip for detecting a mesenchymal stem cell marker gene, wherein at least one of the DNA according to “10” or “11” is immobilized (“12”), the microarray or the DNA chip for detecting a mesenchymal stem cell marker gene according to “12”, wherein a probe for detecting two or more genes in a group of genes having a base sequence shown in SEQ ID NO: 1, 2, 4, 6, 7, 9, 10, 11, 12, 14, 15, 17, 19 or 46 in the sequence listing and a group of the genes according to any one of “2” to “9” is immobilized (“13”), a polypeptide marker for detecting a mesenchymal stem cell which is a polypeptide having an amino acid sequence shown in SEQ ID NO: 3, 5, 8, 13, 16 or 18 in the sequence listing (“14”), an antibody which is induced by using the polypeptide according to “14” and which specifically binds to the polypeptide (“15”), the antibody according to “15”, which is a monoclonal antibody (“16”), and the antibody according to “15”, which is a polyclonal antibody (“17”).

The present invention also comprises: a method for distinguishing a mesenchymal stem cell wherein expression of the mesenchymal stem cell marker gene according to any one of “1” to “9” in a test cell is detected (“18”), the method for distinguishing a mesenchymal stem cell according to “18”, wherein the expression of the mesenchymal stem cell marker gene is detected by using Northern blotting (“19”), the method for distinguishing a mesenchymal stem cell according to “18”, wherein the expression of the mesenchymal stem cell marker gene is detected by using the probe for detecting a mesenchymal stem cell marker gene according to “10” or “11” (“20”), the method for distinguishing a mesenchymal stem cell according to “18”, wherein the expression of the mesenchymal stem cell marker gene is detected by using the microarray or the DNA chip for detecting a mesenchymal stem cell marker gene according to “12” or “13” (“21”), a method for distinguishing a mesenchymal stem cell, wherein the detection of the expression of the mesenchymal stem cell marker gene according to any one of “18” to “21” comprises use of quantitative or semiquantitative PCR (“22”), and the method for distinguishing a mesenchymal stem cell according to “22”, wherein the use of quantitative or semiquantitative PCR is RT-PCR method (“23”).

The present invention further comprises: an RT-PCR primer for amplifying a mesenchymal stem cell marker gene which comprises a sense primer having a base sequence shown in SEQ ID NO: 20 in the sequence listing, and an antisense primer having a base sequence shown in SEQ ID NO: 21 in the sequence listing; a sense primer having a base sequence shown in SEQ ID NO: 22 in the sequence listing, and an antisense primer having a base sequence shown in SEQ ID NO: 23 in the sequence listing; a sense primer having a base sequence shown in SEQ ID NO: 24 in the sequence listing, and an antisense primer having a base sequence shown in SEQ ID NO: 25 in the sequence listing; a sense primer having a base sequence shown in SEQ ID NO: 26 in the sequence listing, and an antisense primer having a base sequence shown in SEQ ID NO: 27 in the sequence listing; a sense primer having a base sequence shown in SEQ ID NO: 28 in the sequence listing, and an antisense primer having a base sequence shown in SEQ ID NO: 29 in the sequence listing; a sense primer having a base sequence shown in SEQ ID NO: 30 in the sequence listing, and an antisense primer having a base sequence shown in SEQ ID NO: 31 in the sequence listing; a sense primer having a base sequence shown in SEQ ID NO: 32 in the sequence listing, and an antisense primer having a base sequence shown in SEQ ID NO: 33 in the sequence listing; a sense primer having a base sequence shown in SEQ ID NO: 34 in the sequence listing, and an antisense primer having a base sequence shown in SEQ ID NO: 35 in the sequence listing; a sense primer having a base sequence shown in SEQ ID NO: 36 in the sequence listing, and an antisense primer having a base sequence shown in SEQ ID NO: 37 in the sequence listing; a sense primer having a base sequence shown in SEQ ID NO: 38 in the sequence listing, and an antisense primer having a base sequence shown in SEQ ID NO: 39 in the sequence listing; a sense primer having a base sequence shown in SEQ ID NO: 40 in the sequence listing, and an antisense primer having a base sequence shown in SEQ ID NO: 41 in the sequence listing; a sense primer having a base sequence shown in SEQ ID NO: 42 in the sequence listing, and an antisense primer having a base sequence shown in SEQ ID NO: 43 in the sequence listing; or a sense primer having a base sequence shown in SEQ ID NO: 44 in the sequence listing, and an antisense primer having a base sequence shown in SEQ ID NO: 45 in the sequence listing (“24”), a real time PCR primer for amplifying a mesenchymal stem cell marker gene which comprises a sense primer having a base sequence shown in SEQ ID NO: 47 in the sequence listing, and an antisense primer having a base sequence shown in SEQ ID NO: 48 in the sequence listing; a sense primer having a base sequence shown in SEQ ID NO: 49 in the sequence listing, and an antisense primer having a base sequence shown in SEQ ID NO: 50 in the sequence listing; a sense primer having a base sequence shown in SEQ ID NO: 51 in the sequence listing, and an antisense primer having a base sequence shown in SEQ ID NO: 52 in the sequence listing; a sense primer having a base sequence shown in SEQ ID NO: 53 in the sequence listing, and an antisense primer having a base sequence shown in SEQ ID NO: 54 in the sequence listing; a sense primer having a base sequence shown in SEQ ID NO: 55 in the sequence listing, and an antisense primer having a base sequence shown in SEQ ID NO: 56 in the sequence listing; a sense primer having a base sequence shown in SEQ ID NO: 57 in the sequence listing, and an antisense primer having a base sequence shown in SEQ ID NO: 58 in the sequence listing; a sense primer having a base sequence shown in SEQ ID NO: 59 in the sequence listing, and an antisense primer having a base sequence shown in SEQ ID NO: 60 in the sequence listing; a sense primer having a base sequence shown in SEQ ID NO: 61 in the sequence listing, and an antisense primer having a base sequence shown in SEQ ID NO: 62 in the sequence listing; a sense primer having a base sequence shown in SEQ ID NO: 63 in the sequence listing, and an antisense primer having a base sequence shown in SEQ ID NO: 64 in the sequence listing; a sense primer having a base sequence shown in SEQ ID NO: 65 in the sequence listing, and an antisense primer having a base sequence shown in SEQ ID NO: 66 in the sequence listing; a sense primer having a base sequence shown in SEQ ID NO: 67 in the sequence listing, and an antisense primer having a base sequence shown in SEQ ID NO: 68 in the sequence listing; or a sense primer having a base sequence shown in SEQ ID NO: 69 in the sequence listing, and an antisense primer having a base sequence shown in SEQ ID NO: 70 in the sequence listing (“25”), and an RT-PCR primer for amplifying a mesenchymal stem cell marker gene which comprises a sense primer having a base sequence shown in SEQ ID NO: 71 in the sequence listing, and an antisense primer having a base sequence shown in SEQ ID NO: 72 in the sequence listing; a sense primer having abase sequence shown in SEQ ID NO: 73 in the sequence listing, and an antisense primer having a base sequence shown in SEQ ID NO: 74 in the sequence listing; a sense primer having a base sequence shown in SEQ ID NO: 75 in the sequence listing, and an antisense primer having a base sequence shown in SEQ ID NO: 76 in the sequence listing; a sense primer having a base sequence shown in SEQ ID NO: 77 in the sequence listing, and an antisense primer having a base sequence shown in SEQ ID NO: 78 in the sequence listing; a sense primer having a base sequence shown in SEQ ID NO: 79 in the sequence listing, and an antisense primer having a base sequence shown in SEQ ID NO: 80 in the sequence listing; a sense primer having a base sequence shown in SEQ ID NO: 81 in the sequence listing, and an antisense primer having a base sequence shown in SEQ ID NO: 82 in the sequence listing; a sense primer having a base sequence shown in SEQ ID NO: 83 in the sequence listing, and an antisense primer having a base sequence shown in SEQ ID NO: 84 in the sequence listing; a sense primer having a base sequence shown in SEQ ID NO: 85 in the sequence listing, and an antisense primer having a base sequence shown in SEQ ID NO: 86 in the sequence listing; a sense primer having a base sequence shown in SEQ ID NO: 87 in the sequence listing, and an antisense primer having a base sequence shown in SEQ ID NO: 88 in the sequence listing; a sense primer having a base sequence shown in SEQ ID NO: 89 in the sequence listing, and an antisense primer having a base sequence shown in SEQ ID NO: 90 in the sequence listing; a sense primer having a base sequence shown in SEQ ID NO: 91 in the sequence listing, and an antisense primer having a base sequence shown in SEQ ID NO: 92 in the sequence listing; a sense primer having a base sequence shown in SEQ ID NO: 93 in the sequence listing, and an antisense primer having a base sequence shown in SEQ ID NO: 94 in the sequence listing; a sense primer having a base sequence shown in SEQ ID NO: 95 in the sequence listing, and an antisense primer having a base sequence shown in SEQ ID NO: 96 in the sequence listing; a sense primer having a base sequence shown in SEQ ID NO: 97 in the sequence listing, and an antisense primer having a base sequence shown in SEQ ID NO: 98 in the sequence listing; a sense primer having a base sequence shown in SEQ ID NO: 99 in the sequence listing, and an antisense primer having a base sequence shown in SEQ ID NO: 100 in the sequence listing; a sense primer having a base sequence shown in SEQ ID NO: 101 in the sequence listing, and an antisense primer having a base sequence shown in SEQ ID NO: 102 in the sequence listing; a sense primer having a base sequence shown in SEQ ID NO: 103 in the sequence listing, and an antisense primer having a base sequence shown in SEQ ID NO: 104 in the sequence listing; a sense primer having a base sequence shown in SEQ ID NO: 105 in the sequence listing, and an antisense primer having a base sequence shown in SEQ ID NO: 106 in the sequence listing; a sense primer having a base sequence shown in SEQ ID NO: 107 in the sequence listing, and an antisense primer having a base sequence shown in SEQ ID NO: 108 in the sequence listing; a sense primer having a base sequence shown in SEQ ID NO: 109 in the sequence listing, and an antisense primer having a base sequence shown in SEQ ID NO: 110 in the sequence listing; a sense primer having a base sequence shown in SEQ ID NO: 111 in the sequence listing, and an antisense primer having a base sequence shown in SEQ ID NO: 112 in the sequence listing; a sense primer having a base sequence shown in SEQ ID NO: 113 in the sequence listing, and an antisense primer having a base sequence shown in SEQ ID NO: 114 in the sequence listing; or a sense primer having a base sequence shown in SEQ ID NO: 115 in the sequence listing, and an antisense primer having a base sequence shown in SEQ ID NO: 116 in the sequence listing (“26”). The present invention still further comprises: the method for distinguishing a mesenchymal stem cell according to “18”, wherein a gene in a test cell is amplified by using at least one pair of primers comprising the sense primer and the antisense primer according to any one of “24” to “26”, and the amplified gene is detected by using the probe for detecting a mesenchymal stem cell marker gene according to “10” or “11” (“27”), a method for distinguishing a mesenchymal stem cell, wherein expression of a mesenchymal stem cell marker polypeptide in a test cell is detected by using the antibody according to any one of “15” to “17” (“28”), a method for distinguishing a mesenchymal stem cell, wherein expression of one or more genes in a group of mesenchymal stem cell marker genes comprising SEQ ID NOs: 1, 11 and 19 in the sequence listing, and expression of one or more genes in a group of mesenchymal stem cell marker genes comprising SEQ ID NOs: 2, 4, 6, 7, 9, 10, 12, 14, 15 and 17 in the sequence listing, in a test cell, are evaluated in combination (“29”), a method for distinguishing and separating a mesenchymal stem cell, wherein expression of a marker gene for detecting a mesenchymal stem cell and/or a marker polypeptide for detecting a mesenchymal stem cell in a test cell is detected by using the probe for detecting a mesenchymal stem cell marker gene according to “10” or “11” and/or the antibody according to any one of “15” to “17” and the distinguished mesenchymal stem cell is separated (“30”), the method for distinguishing and separating a mesenchymal stem cell according to “30”, wherein a mesenchymal stem cell in a test cell is labeled by a fluorescent antibody method using the antibody according to any one of “15” to “17”, and the labeled mesenchymal stem cell is separated (“31”), and a kit for distinguishing a mesenchymal stem cell which comprises at least one of the probe for detecting a mesenchymal stem cell marker gene according to “10” or “11”, the microarray or the DNA chip for detecting a mesenchymal stem cell marker gene according to “12” or “13” in which the probe is immobilized, and the antibody according to any one of “15” to “17” (“32”).

### BRIEF DESCRIPTION OF DRAWINGS

FIG. 1 shows genes exhibiting considerable individual differences between human mesenchymal stem cells or human fibroblasts, according to the semiquantitative RT-PCR method in the Examples of the present invention.

FIG. 2 shows genes exhibiting high expression in MSC, according to the semiquantitative RT-PCR in the Examples of the present invention.

FIG. 3 shows genes exhibiting low expression in MSC, according to the semiquantitative RT-PCR in the Examples of the present invention.

FIG. 4 shows the result of confirmation of the expression levels of 13 genes confirmed to exhibit high/low expression in mesenchymal stem cells, by using semiquantitative RT-PCR with total RNA extracted from mesenchymal stem cells derived from 7 individuals and fibroblasts derived from 4 individuals as a template.

FIG. 5 shows the test result of the expression state of the marker gene of the present invention in mesenchymal stem cells and fibroblasts in the Examples of the present invention.

FIG. 6 shows the expression state of differential gene of each marker gene in mesenchymal stem cells and osteoblasts in the Examples of the present invention.

FIG. 7 shows the expression state of differential gene of each marker gene in mesenchymal stem cells and osteoblasts in the Examples of the present invention.

FIG. 8 shows the expression state of differential gene of each marker gene in mesenchymal stem cells and osteoblasts in the Examples of the present invention.

FIG. 9 shows the expression state of each marker gene in mesenchymal stem cells and human fibroblasts in the Examples of the present invention.

FIG. 10 shows the expression state of each marker gene in mesenchymal stem cells and human fibroblasts in the Examples of the present invention.

FIG. 11 shows the result of flow cytometric measurement of the expression of mesenchymal stem cell DR in human cells in the Examples of the present invention.

FIG. 12 shows the measurement result of the expression state of each gene in inducing differentiation of mesenchymal stem cells into osteoblasts in the Examples of the present invention.

FIG. 13 shows the changes in gene expression of each marker gene from day 4 to day 28 in the induction of osteogenic differentiation in the Examples of the present invention.

FIG. 14 shows the changes in gene expression of each marker gene from day 4 to day 28 in the induction of osteogenic differentiation in the Examples of the present invention.

FIG. 15 shows the changes in gene expression of each marker gene from day 4 to day 28 in the induction of osteogenic differentiation in the Examples of the present invention.

FIG. 16 shows the changes in gene expression of each marker gene from day 4 to day 28 in the induction of osteogenic differentiation in the Examples of the present invention.

### BEST MODE OF CARRYING OUT THE INVENTION

The present invention comprises distinguishing mesenchymal stem cells by detecting the expression of a marker gene for detecting mesenchymal stem cells or a marker polypeptide for detecting mesenchymal stem cells, which are expressed specifically in mesenchymal stem cells. In the present invention, a gene which can serve as a gene marker for detecting mesenchymal stem cells is a gene having a base sequence shown in SEQ ID NO: 1, 2, 4, 6, 7, 9, 10, 11, 12, 14, 15, 17 or 19 in the sequence listing.

Further, in the present invention, a polypeptide which serves as a polypeptide marker for detecting mesenchymal stem cells is a polypeptide having an amino acid sequence shown in SEQ ID NO: 3, 5, 8, 13, 16 or 18 in the sequence listing.

The genes shown in SEQ ID NOs: 1, 2, 4, 6, 7, 9, 10, 11, 12, 14, 15, 17, 19 and 46 in the sequence listing are genes of [serine (or cysteine) proteinase inhibitor], [adrenomedullin], [apolopoprotein D], [collagenase type XV alpha 1], [CUG triplet repeat RNA binding protein2], [dermatopontin], [isocitrate dehydrogenase 2], [major histocompatibility complex class2, DR beta 3], [protein tyrosine kinase 7], [Sam68-like phosphotyrosine protein], [C-type lectin superfamily member 2], [matrix metalloprotease 1], [tissue factor pathway inhibitor 2], [major histocompatibility complex DR alpha], respectively.

The DNA sequence information of the marker genes for detecting mesenchymal stem cells of the present invention can be approached in NCBI gene database with Accession Nos. AI133613(SEQ ID NO: 1), NM_{—}001124(SEQ ID NO: 2), NM_{—}001647(SEQ ID NO: 4), L01697(SEQ ID NO: 6), U69546(SEQ ID NO: 7), AW016451(SEQ ID NO: 9), AL545953(SEQ ID NO: 10), BF732822(SEQ ID NO: 11), AL157486(SEQ ID NO: 12), AA112001(SEQ ID NO: 14), XM_{—}006626(SEQ ID NO: 15), NM_{—}002421(SEQ ID NO: 17), AL550357(SEQ ID NO: 19), and BF795929(SEQ ID NO: 46), respectively.

In addition, as genes which are confirmed in the present invention that they exhibit higher expression in mesenchymal stem cells than in fibroblasts, the following is exemplified:

A gene marker for detecting mesenchymal stem cells which is: a gene of INTEGRIN, ALPHA 6 (ITGA6), MRNA (NM_{—}000210); a gene of SOLUTE CARRIER FAMILY 20 (PHOSPHATE TRANSPORTER), MEMBER 1 (NM_{—}005415); a gene of RIBONUCLEOTIDE REDUCTASE M2 POLYPEPTIDE (RRM2), MRNA (NM 001034); a gene of FOLLISTATIN (FST), TRANSCRIPT VARIANT FST317, MRNA (NM_{—}006350); a gene of SPROUTY (DROSOPHILA) HOMOLOG 2 (SPRY2), MRNA (NM_{—}005842); a gene of RAB3B, MEMBER RAS ONCOGENE FAMILY (RAB3B), MRNA (NM_{—}002867); a gene of SOLUTE CARRIER FAMILY 2 (FACILITATED GLUCOSE TRANSPORTER) (NM_{—}006516); a gene of INTERLEUKIN 13 RECEPTOR, ALPHA 2 (IL13RA2), MRNA (NM_{—}000640); a gene of SERINE/THREONINE KINASE 12 (STK12), MRNA (NM_{—}004217); a gene of MINICHROMOSOME MAINTENANCE DEFICIENT (S. CEREVISIAE) 5 (CE) (NM_{—}006739); a gene of THYROID HORMONE RECEPTOR INTERACTOR 13 (TRIP13), MRNA (NM_{—}004237); a gene of KINESIN-LIKE 6 (MITOTIC CENTROMERE-ASSOCIATED KINESIN) (K) (NM_{—}006845); a gene of CYCLIN-DEPENDENT KINASE INHIBITOR 3 (CDK2-ASSOCIATED DUAL (NM_{—}005192); a gene of CHROMOSOME CONDENSATION PROTEIN G (HCAP-G), MRNA (NM_{—}022346); a gene of CDC28 PROTEIN KINASE 1 (CKS1), MRNA (NM_{—}001826); a gene of PROTEIN REGULATOR OF CYTOKINESIS 1 (PRC1), MRNA (NM_{—}003981); a gene of CELL DIVISION CYCLE 2, G1 TO S AND G2 TOM (CDC2), MRNA (NM_{—}001786); a gene of CDC20 (CELL DIVISION CYCLE 20, S. CEREVISIAE, HOMOLOG) (CD) (NM_{—}001255); a gene of LIKELY ORTHOLOG OF MATERNAL EMBRYONIC LEUCINE ZIPPER KINAS (NM_{—}014791); a gene of MINICHROMOSOME MAINTENANCE DEFICIENT (S. CEREVISIAE) 7 (M) (NM_{—}005916); a gene of CYCLIN A2 (CCNA2), MRNA (NM_{—}001237); a gene of THYMIDINE KINASE 1, SOLUBLE (TK1), MRNA (NM_{—}003258); or a gene of cyclin-dependent kinase inhibitor 2A (melanoma, p16, inhibits CDK4) (CDKN2A), mRNA (NM_{—}000077).

A gene of EGF-containing fibulin-like extracellular matrix protein 1 (NM_{—}018894); a gene of Human insulin-like growth factor binding protein 5 (IGFBP5) mRNA (AU132011); a gene of Homo sapiens clone 24775 mRNA sequence (AA402981); proteoglycan 1, secretory granule (AV734015); a gene of insulin-like growth factor binding protein 5 (AA374325); a gene of solute carrier family 21 (organic anion transporter), member 3 (AF085224); a gene of transglutaminase 2 (C polypeptide, protein-glutamine-gamma-glutamyl transferase) (AL552373); a gene of coagulation factor II (thrombin) receptor (M62424); a gene of plasminogen activator, urokinase (NM_{—}002658); a gene of tissue inhibitor of metalloproteinase 3 (Sorsby fundus dystrophy, pseudoinflammatory) (W96324); a gene of matrix Gla protein (BF668572); a gene of B-cell CLL/lymphoma 1(Z23022); a gene of CD74 antigen (invariant polypeptide of major histocompatibility complex, class II antigen-associated) (BG333618); a gene of adducin 3 (gamma) (AL135243); a gene of solute carrier family 16 (monocarboxylic acid transporters), member 4 (NM_{—}004696); a gene of protein S (alpha) (NM_{—}000313); a gene of phosphatidylinositol (4,5) bisphosphate 5-phosphatase homolog; phosphatidylinositol polyphosphate 5-phosphatase type IV(AF187891); a gene of progesterone membrane binding protein (BE858855); a gene of brain-derived neurotrophic factor (X60201); or a gene of apolipoprotein E (BF967316).

A gene of HOMO SAPIENS WNT INHIBITORY FACTOR-1 (WIF-1), MRNA (NM_{—}007191); a gene of HOMO SAPIENS HYPOTHETICAL PROTEIN FLJ11175 (FLJ11175), MRNA(NM_{—}018349); a gene of HOMO SAPIENS RHO GDP DISSOCIATION INHIBITOR (GDI) BETA (ARHGDIB), MRNA (NM_{—}001175); a gene of HOMO SAPIENS POTASSIUM INTERMEDIATE/SMALL CONDUCTANCE CALCIUM-ACTIVATE(NM_{—}021614); a gene of HOMO SAPIENS NUCLEAR FACTOR (ERYTHROID-DERIVED 2)-LIKE 3 (NFE2L3), MRNA (NM_{—}004289); a gene of HOMO SAPIENS GS3955 PROTEIN (GS3955), MRNA (NM_{—}021643); a gene of HOMO SAPIENS CDNA 3′ END SIMILAR TO SIMILAR TO GLYCOPROTEIN MUC18 (AA302605); a gene of HOMO SAPIENS HYPOTHETICAL PROTEIN FLJ21841 (FLJ21841), MRNA (NM_{—}024609); a gene of HOMO SAPIENS ZINC FINGER PROTEIN 185 (LIM DOMAIN) (ZNF185), MRNA (NM_{—}007150); a gene of SC1=PUTATIVE TRANS-ACTING FACTOR INVOLVED IN CELL CYCLE CONTROL [HUMAN, MRNA, 24] (S53374); a gene of HOMO SAPIENS NADH DEHYDROGENASE (UBIQUINONE) 1 ALPHA SUBCOMPLEX, 8 (19K) (NM_{—}014222); a gene of HOMO SAPIENS HOMOLOG OF YEAST MCM10; HYPOTHETICAL PROTEIN PRO2249 (PRO2) (NM_{—}018518); a gene of HOMO SAPIENS COLLAGEN, TYPE VII, ALPHA 1 (EPIDERMOLYSIS BULLOSA, DYSTRO (NM_{—}000094); a gene of HOMO SAPIENS AUTOCRINE MOTILITY FACTOR RECEPTOR (AMFR), MRNA (NM_{—}001144); a gene of HOMO SAPIENS CLONE 24421 MRNA SEQUENCE /CDS=UNKNOWN/GB=AF070641/GI=3283914/UG=(AF070641); a gene of HOMO SAPIENS MRNA; a gene of CDNA DKFZP586E1621 (FROM CLONE DKFZP586E1621)/CDS=UNKNOWN/GB(AL080235).

A gene of HOMO SAPIENS RESERVED (KCNK12), MRNA (NM_{—}022055); a gene of HOMO SAPIENS UBIQUITIN-CONJUGATING ENZYME E2C (UBE2C), MRNA (NM_{—}007019); a gene of HOMO SAPIENS CDNA FLJ10517 FIS, CLONE NT2RP2000812/CDS=(61,918)/GB=AK001379/G(AK001379); a gene of HOMO SAPIENS CARTILAGE LINKING PROTEIN 1 (CRTL1), MRNA (NM_{—}001884); a gene of HOMO SAPIENS MRNA; CDNA DKFZP434F2322 (FROM CLONE DKFZP434F2322)/CDS=UNKNOWN/GB (AL133105); a gene of HOMO SAPIENS, CLONE MGC:9549 IMAGE:3857382, MRNA, COMPLETE CDS/CDS=(92,1285)/G (BC012453); a gene of HOMO SAPIENS HYPOTHETICAL PROTEIN DKFZP586J1119 (DKFZP586J1119), MRNA (NM_{—}032270); a gene of HOMO SAPIENS GLIA MATURATION FACTOR, GAMMA (GMFG), MRNA (NM_{—}004877); a gene of HOMO SAPIENS CDNA: FLJ23095 FIS, CLONE LNG07413/CDS=UNKNOWN/GB=AK026748/GI=10 (AK026748); a gene of HOMO SAPIENS HYPOTHETICAL PROTEIN DKFZP762E1312 (DKFZP762E1312), MRNA (NM_{—}018410); a gene of HOMO SAPIENS IROQUOIS-CLASS HOMEODOMAIN PROTEIN (IRX-2A), MRNA (NM_{—}005853); or a gene of HOMO SAPIENS HYPOTHETICAL PROTEIN FLJ10540 (FLJ10540), MRNA (NM_{—}018131).

A gene of HOMO SAPIENS MRNA; CDNA DKFZP434C1915 (FROM CLONE DKFZP434C1915); PARTIAL CDS/C (AL137698); a gene of HOMO SAPIENS MINICHROMOSOME MAINTENANCE DEFICIENT (S. CEREVISIAE) 7 (M) (NM_{—}005916); a gene of HOMO SAPIENS DYNEIN, CYTOPLASMIC, INTERMEDIATE POLYPEPTIDE 1 (DNCI1), M (NM_{—}004411); a gene of HUMAN MRNA FOR PROTEIN GENE PRODUCT (PGP) 9.5. (X04741); a gene of HOMO SAPIENS RAD51-INTERACTING PROTEIN (PIR51), MRNA (NM_{—}006479); a gene of HOMO SAPIENS BACULOVIRAL IAP REPEAT-CONTAINING 5 (SURVIVIN) (BIRC5), MR (NM_{—}001168); a gene of HOMO SAPIENS UDP-N-ACETYL-ALPHA-D-GALACTOSAMINE: POLYPEPTIDE N-ACETYLGAL (NM_{—}004482); a gene of HOMO SAPIENS CYCLIN B1 (CCNB1), MRNA (NM_{—}031966); a gene of HOMO SAPIENS FLAP STRUCTURE-SPECIFIC ENDONUCLEASE 1 (FEN1), MRNA (NM_{—}004111); a gene of HOMO SAPIENS SERINE/THREONINE KINASE 15 (STK15), MRNA (NM_{—}003600); a gene of HOMO SAPIENS MAD2 (MITOTIC ARREST DEFICIENT, YEAST, HOMOLOG)-LIKE 1 (MA) (NM_{—}002358); a gene of HOMO SAPIENS NUCLEOLAR PROTEIN ANKT (ANKT), MRNA (NM_{—}018454); a gene of HOMO SAPIENS HSPC150 PROTEIN SIMILAR TO UBIQUITIN-CONJUGATING ENZYME (H) (NM_{—}014176); a gene of HOMO SAPIENS CYTOCHROME P450 RETINOID METABOLIZING PROTEIN (P450RAI-2), (NM_{—}019885); a gene of HOMO SAPIENS NIMA (NEVER IN MITOSIS GENE A)-RELATED KINASE 2 (NEK2), MR (NM_{—}002497); a gene of HOMO SAPIENS BONE MORPHOGENETIC PROTEIN 4 (BMP4), MRNA (NM_{—}001202); a gene of HOMO SAPIENS CDNA FLJ10674 FIS, CLONE NT2RP2006436/CDS=UNKNOWN/GB=AK001536/GI (AK001536); or a gene of HOMO SAPIENS CENTROMERE PROTEIN A (17 KD) (CENPA), MRNA (NM_{—}001809).

A gene of HOMO SAPIENS MRNA; CDNA DKFZP564P116 (FROM CLONE DKFZP564P116)/CDS=UNKNOWN/GB=A (AL049338); a gene of HOMO SAPIENS TTK PROTEIN KINASE (TTK), MRNA (NM_{—}003318); a gene of HOMO SAPIENS KARYOPHERIN ALPHA 2 (RAG COHORT 1, IMPORTIN ALPHA 1) (KPNA (NM_{—}002266); a gene of HOMO SAPIENS POLYMERASE (DNA DIRECTED), THETA (POLQ), MRNA (NM 006596); a gene of HOMO SAPIENS ETS VARIANT GENE 5 (ETS-RELATED MOLECULE) (ETV5), MRNA (NM_{—}004454); a gene of HOMO SAPIENS TRANSFORMING, ACIDIC COILED-COIL CONTAINING PROTEIN 3 (TAC) (NM_{—}006342); a gene of HOMO SAPIENS KINESIN-LIKE 1 (KNSL1), MRNA (NM_{—}004523); a gene of HOMO SAPIENS CENTROMERE PROTEIN E (312 KD) (CENPE), MRNA (NM_{—}001813); a gene of HOMO SAPIENS CDNA, 3′ END/CLONE=IMAGE:1651289/CLONE_END=3′/GB=AI12 (AI123815); a gene of HOMO SAPIENS DISTAL-LESS HOMEO BOX 5 (DLX5), MRNA (NM_{—}005221); a gene of HOMO SAPIENS V-MYB AVIAN MYELOBLASTOSIS VIRAL ONCOGENE HOMOLOG-LIKE 2 (NM_{—}002466); or a gene of HOMO SAPIENS SERUM DEPRIVATION RESPONSE (PHOSPHATIDYLSERINE-BINDING PRO (NM_{—}004657).

A gene of HOMO SAPIENS HYPOTHETICAL PROTEIN FLJ10604 (FLJ10604), MRNA (NM_{—}018154); a gene of HOMO SAPIENS RIBONUCLEASE HI, LARGE SUBUNIT (RNASEHI), MRNA (NM_{—}006397); a gene of HOMO SAPIENS ANILLIN (DROSOPHILA SCRAPS HOMOLOG), ACTIN BINDING PROTEIN (NM_{—}018685); a gene of HOMO SAPIENS H4 HISTONE FAMILY, MEMBER G (H4FG), MRNA (NM_{—}003542); a gene of HOMO SAPIENS G PROTEIN-COUPLED RECEPTOR 37 (ENDOTHELIN RECEPTOR TYPE B-(NM 005302) HOMO SAPIENS UBIQUITIN CARRIER PROTEIN (E2-EPF), MRNA (NM_{—}014501); a gene of HOMO SAPIENS, SIMILAR TO TUMOR DIFFERENTIALLY EXPRESSED 1, CLONE IMAGE:3639252, (BC007375); a gene of HOMO SAPIENS HYPOTHETICAL PROTEIN MGC2577 (MGC2577), MRNA(NM_{—}031299); a gene of HOMO SAPIENS HYPOTHETICAL PROTEIN FLJ13912 (FLJ13912), MRNA (NM_{—}022770); a gene of HOMO SAPIENS ANNEXIN A3 (ANXA3), MRNA (NM_{—}005139); a gene of HOMO SAPIENS STATHMIN 1/ONCOPROTEIN 18 (STMN1), MRNA (NM_{—}005563) HOMO SAPIENS, SIMILAR TO RIBOSOMAL PROTEIN L39, CLONE MGC:20168 IMAGE:4555759, M (BC012328); a gene of HOMO SAPIENS POLO (DROSOPHIA)-LIKE KINASE (PLK), MRNA (NM_{—}005030).

Further, as genes which are confirmed in the present invention that they exhibit no or extremely lower expression in mesenchymal stem cells than in fibroblasts, the following is exemplified:

A gene marker for detecting mesenchymal stem cells which is: a gene of HOMO SAPIENS PROTEASOME (PROSOME, MACROPAIN) 26S SUBUNIT, ATPASE, 5 (PS) (NM_{—}002805); a gene of HOMO SAPIENS EUKARYOTIC TRANSLATION INITIATION FACTOR 4 GAMMA, 2 (EIF4G) (NM_{—}001418); a gene of HOMO SAPIENS DIHYDROPYRIMIDINASE-LIKE 3 (DPYSL3), MRNA) (NM_{—}001387); a gene of HOMO SAPIENS ADAPTOR-RELATED PROTEIN COMPLEX 1, SIGMA 2 SUBUNIT (AP1S2) (NM_{—}003916); a gene of HOMO SAPIENS PLECTIN 1, INTERMEDIATE FILAMENT BINDING PROTEIN, 500 KD (P) (NM_{—}000445); a gene of HOMO SAPIENS HETEROGENEOUS NUCLEAR RIBONUCLEOPROTEIN U (SCAFFOLD ATTAC (NM_{—}031844); a gene of HOMO SAPIENS HYPOTHETICAL PROTEIN MGC5306 (MGC5306), MRNA (NM_{—}024116); a gene of HOMO SAPIENS MHC CLASS I POLYPEPTIDE-RELATED SEQUENCE A (MICA), MRNA (NM_{—}000247); a gene of HOMO SAPIENS PROTEIN ASSOCIATED WITH PRK1 (AWP1), MRNA (NM_{—}019006); a gene of HOMO SAPIENS PROGESTERONE RECEPTOR MEMBRANE COMPONENT 2 (PGRMC2), MRNA (NM_{—}006320); a gene of HOMO SAPIENS POLYGLUTAMINE BINDING PROTEIN 1 (PQBP1), MRNA (NM_{—}005710); a gene of HOMO SAPIENS S-ADENOSYLMETHIONINE DECARBOXYLASE 1 (AMD1), MRNA (NM_{—}001634); HOMO SAPIENS INTERFERON—INDUCED, HEPATITIS C-ASSOCIATED MICROTUBULAR AG (NM_{—}006417); a gene of HOMO SAPIENS PROTEIN KINASE, AMP-ACTIVATED, GAMMA 1 NON-CATALYTIC SUBUN (NM_{—}002733); HOMO SAPIENS PROCOLLAGEN-PROLINE, 2-OXOGLUTARATE 4-DIOXYGENASE (PROLINE (NM_{—}004199); a gene of HOMO SAPIENS ENDOTHELIAL DIFFERENTIATION, LYSOPHOSPHATIDIC ACID G-PROTE (NM_{—}012152); or a gene of HOMO SAPIENS KIAA0127 GENE PRODUCT (KIAA0127), MRNA(NM_{—}014755).

In the present invention, known methods for detecting gene expression can be used to detect the expression of the mesenchymal stem cell marker gene of the present invention. For example, Northern blotting can be used to detect the expression of the mesenchymal stem cell marker gene of the present invention. In addition, it is possible to use a probe having a DNA sequence which hybridizes with a DNA sequence of the mesenchymal stem cell gene marker of the present invention under a stringent condition in order to detect and distinguish mesenchymal stem cells by the gene marker of the present invention. For the detection of mesenchymal stem cells by using the probe, known methods can be appropriately used. For instance, the detection of mesenchymal stem cells is conducted by the following process: constructing a DNA probe with appropriate length from a DNA sequence of a gene marker shown in the sequence listing; labeling the probe appropriately by fluorescent labeling, etc.; hybridizing the probe with a test substance. As the DNA probe, it is possible to use a probe for detecting mesenchymal stem cell marker genes comprising whole or part of an antisense strand of the base sequence of the gene marker of the present invention shown in the sequence listing. The probe can be used also in a form of a microarray or a DNA chip for detecting marker genes, wherein at least one of the probes is immobilized.

In the construction of the DNA probe mentioned above, the condition for the base sequence of the present invention “to hybridize with a DNA sequence of a marker gene for detecting mesenchymal stem cells under a stringent condition” is exemplified by hybridization at 42° C., and washing treatment at 42° C. with buffer solution containing 1×SSC (0.15 M NaCl, 0.015 M sodium citrate), and 0.1% SDS (sodium dodecyl sulfate), and more preferably exemplified by hybridization at 65° C., and washing treatment at 65° C. with buffer solution containing 0.1×SSC, and 0.1% SDS. There are various factors other than the temperature conditions mentioned above that affect the hybridization stringency and those skilled in the art can actualize the same stringency as that of the hybridization referred to in the above by appropriately combining various factors.

Further, when detecting the polypeptide marker for detecting mesenchymal stem cells of the present invention, an antibody induced by the polypeptide of the protein and specifically binds to the polypeptide can be used in the present invention. Examples of the antibody include monoclonal and polyclonal antibodies. The antibody can be constructed by ordinary methods with the polypeptide marker of the present invention as an antigen. In order to detect the expression of the marker polypeptide for detecting mesenchymal stem cells of the present invention in a test cell by using the antigen of the present invention, immunoassay methods using known antibodies can be used. As for such immunoassay methods, known immunoassay methods such as RIA, ELISA, and fluorescent antibody method are exemplified.

In the present invention, it is possible to detect and distinguish the expression of a marker gene for detecting mesenchymal stem cells and/or a marker polypeptide for detecting mesenchymal stem cells in a test cell by using the probe for detecting mesenchymal stem cells of the present invention and/or the antibody for detecting mesenchymal stem cells of the present invention, and to separate and obtain mesenchymal stem cells. When detecting a marker gene for detecting mesenchymal stem cells in a test cell, quantitative or semiquantitative PCR can be used to amplify the gene of the test cell. RT-PCR (reverse transcription PCR) can be used as the PCR. When conducting the PCR, primers comprising a sense primer and an antisense primer for amplifying a marker gene for detecting mesenchymal stem cells of the present invention are used.

Examples of the primers for amplifying a marker gene for detecting mesenchymal stem cells of the present invention include: a sense primer having a base sequence shown in SEQ ID NO: 20 in the sequence listing, and an antisense primer having a base sequence shown in SEQ ID NO: 21 in the sequence listing; a sense primer having a base sequence shown in SEQ ID NO: 22 in the sequence listing, and an antisense primer having a base sequence shown in SEQ ID NO: 23 in the sequence listing; a sense primer having a base sequence shown in SEQ ID NO: 24 in the sequence listing, and an antisense primer having a base sequence shown in SEQ ID NO: 25 in the sequence listing; a sense primer having a base sequence shown in SEQ ID NO: 26 in the sequence listing, and an antisense primer having a base sequence shown in SEQ ID NO: 27 in the sequence listing; a sense primer having a base sequence shown in SEQ ID NO: 28 in the sequence listing, and an antisense primer having a base sequence shown in SEQ ID NO: 29 in the sequence listing; a sense primer having a base sequence shown in SEQ ID NO: 30 in the sequence listing, and an antisense primer having a base sequence shown in SEQ ID NO: 31 in the sequence listing; a sense primer having a base sequence shown in SEQ ID NO: 32 in the sequence listing, and an antisense primer having a base sequence shown in SEQ ID NO: 33 in the sequence listing; a sense primer having a base sequence shown in SEQ ID NO: 34 in the sequence listing, and an antisense primer having a base sequence shown in SEQ ID NO: 35 in the sequence listing; a sense primer having a base sequence shown in SEQ ID NO: 36 in the sequence listing, and an antisense primer having a base sequence shown in SEQ ID NO: 37 in the sequence listing; a sense primer having a base sequence shown in SEQ ID NO: 38 in the sequence listing, and an antisense primer having a base sequence shown in SEQ ID NO: 39 in the sequence listing; a sense primer having a base sequence shown in SEQ ID NO: 40 in the sequence listing, and an antisense primer having a base sequence shown in SEQ ID NO: 41 in the sequence listing; a sense primer having a base sequence shown in SEQ ID NO: 42 in the sequence listing, and an antisense primer having a base sequence shown in SEQ ID NO: 43 in the sequence listing; or a sense primer having a base sequence shown in SEQ ID NO: 44 in the sequence listing, and an antisense primer having a base sequence shown in SEQ ID NO: 45 in the sequence listing.

Examples of the primers for amplifying a marker gene for detecting mesenchymal stem cells of the present invention further include: a real time PCR primer for amplifying a mesenchymal stem cell marker gene which comprises a sense primer having a base sequence shown in SEQ ID NO: 47 in the sequence listing, and an antisense primer having a base sequence shown in SEQ ID NO: 48 in the sequence listing; a sense primer having a base sequence shown in SEQ ID NO: 49 in the sequence listing, and an antisense primer having a base sequence shown in SEQ ID NO: 50 in the sequence listing; a sense primer having a base sequence shown in SEQ ID NO: 51 in the sequence listing, and an antisense primer having a base sequence shown in SEQ ID NO: 52 in the sequence listing; a sense primer having a base sequence shown in SEQ ID NO: 53 in the sequence listing, and an antisense primer having a base sequence shown in SEQ ID NO: 54 in the sequence listing; a sense primer having a base sequence shown in SEQ ID NO: 55 in the sequence listing, and an antisense primer having a base sequence shown in SEQ ID NO: 56 in the sequence listing; a sense primer having a base sequence shown in SEQ ID NO: 57 in the sequence listing, and an antisense primer having a base sequence shown in SEQ ID NO: 58 in the sequence listing; a sense primer having a base sequence shown in SEQ ID NO: 59 in the sequence listing, and an antisense primer having a base sequence shown in SEQ ID NO: 60 in the sequence listing; a sense primer having a base sequence shown in SEQ ID NO: 61 in the sequence listing, and an antisense primer having a base sequence shown in SEQ ID NO: 62 in the sequence listing; a sense primer having a base sequence shown in SEQ ID NO: 63 in the sequence listing, and an antisense primer having a base sequence shown in SEQ ID NO: 64 in the sequence listing; a sense primer having a base sequence shown in SEQ ID NO: 65 in the sequence listing, and an antisense primer having a base sequence shown in SEQ ID NO: 66 in the sequence listing; a sense primer having a base sequence shown in SEQ ID NO: 67 in the sequence listing, and an antisense primer having a base sequence shown in SEQ ID NO: 68 in the sequence listing; or a sense primer having a base sequence shown in SEQ ID NO: 69 in the sequence listing, and an antisense primer having a base sequence shown in SEQ ID NO: 70 in the sequence listing.

Examples of the primers for amplifying a marker gene for detecting mesenchymal stem cells of the present invention further include: an RT-PCR primer for amplifying a mesenchymal stem cell marker gene which comprises a sense primer having a base sequence shown in SEQ ID NO: 71 in the sequence listing, and an antisense primer having a base sequence shown in SEQ ID NO: 72 in the sequence listing; a sense primer having a base sequence shown in SEQ ID NO: 73 in the sequence listing, and an antisense primer having a base sequence shown in SEQ ID NO: 74 in the sequence listing; a sense primer having a base sequence shown in SEQ ID NO: 75 in the sequence listing, and an antisense primer having a base sequence shown in SEQ ID NO: 76 in the sequence listing; a sense primer having a base sequence shown in SEQ ID NO: 77 in the sequence listing, and an antisense primer having a base sequence shown in SEQ ID NO: 78 in the sequence listing; a sense primer having a base sequence shown in SEQ ID NO: 79 in the sequence listing, and an antisense primer having a base sequence shown in SEQ ID NO: 80 in the sequence listing; a sense primer having a base sequence shown in SEQ ID NO: 81 in the sequence listing, and an antisense primer having a base sequence shown in SEQ ID NO: 82 in the sequence listing; a sense primer having a base sequence shown in SEQ ID NO: 83 in the sequence listing, and an antisense primer having a base sequence shown in SEQ ID NO: 84 in the sequence listing; a sense primer having a base sequence shown in SEQ ID NO: 85 in the sequence listing, and an antisense primer having a base sequence shown in SEQ ID NO: 86 in the sequence listing; a sense primer having a base sequence shown in SEQ ID NO: 87 in the sequence listing, and an antisense primer having a base sequence shown in SEQ ID NO: 88 in the sequence listing; a sense primer having a base sequence shown in SEQ ID NO: 89 in the sequence listing, and an antisense primer having a base sequence shown in SEQ ID NO: 90 in the sequence listing; a sense primer having a base sequence shown in SEQ ID NO: 91 in the sequence listing, and an antisense primer having a base sequence shown in SEQ ID NO: 92 in the sequence listing; a sense primer having a base sequence shown in SEQ ID NO: 93 in the sequence listing, and an antisense primer having a base sequence shown in SEQ ID NO: 94 in the sequence listing; a sense primer having a base sequence shown in SEQ ID NO: 95 in the sequence listing, and an antisense primer having a base sequence shown in SEQ ID NO: 96 in the sequence listing; a sense primer having a base sequence shown in SEQ ID NO: 97 in the sequence listing, and an antisense primer having a base sequence shown in SEQ ID NO: 98 in the sequence listing; a sense primer having a base sequence shown in SEQ ID NO: 99 in the sequence listing, and an antisense primer having a base sequence shown in SEQ ID NO: 100 in the sequence listing; a sense primer having a base sequence shown in SEQ ID NO: 101 in the sequence listing, and an antisense primer having a base sequence shown in SEQ ID NO: 102 in the sequence listing; a sense primer having a base sequence shown in SEQ ID NO: 103 in the sequence listing, and an antisense primer having a base sequence shown in SEQ ID NO: 104 in the sequence listing; a sense primer having a base sequence shown in SEQ ID NO: 105 in the sequence listing, and an antisense primer having a base sequence shown in SEQ ID NO: 106 in the sequence listing; a sense primer having a base sequence shown in SEQ ID NO: 107 in the sequence listing, and an antisense primer having a base sequence shown in SEQ ID NO: 108 in the sequence listing; a sense primer having a base sequence shown in SEQ ID NO: 109 in the sequence listing, and an antisense primer having a base sequence shown in SEQ ID NO: 110 in the sequence listing; a sense primer having a base sequence shown in SEQ ID NO: 111 in the sequence listing, and an antisense primer having a base sequence shown in SEQ ID NO: 112 in the sequence listing; a sense primer having a base sequence shown in SEQ ID NO: 113 in the sequence listing, and an antisense primer having a base sequence shown in SEQ ID NO: 114 in the sequence listing; or a sense primer having a base sequence shown in SEQ ID NO: 115 in the sequence listing, and an antisense primer having a base sequence shown in SEQ ID NO: 116 in the sequence listing.

In order to distinguish mesenchymal stem cells in the present invention, a gene in a test cell is amplified by using at least one pair of primers comprising the sense primer and the antisense primer mentioned above, and the amplified gene is detected by using the probe for detecting mesenchymal stem cell marker genes of the present invention.

With regard to the marker genes for detecting mesenchymal stem cells of the present invention, some genes exhibit specifically high expression and some genes exhibit specifically low expression in mesenchymal stem cells. By detecting the expressions of these genes in combination, accuracy in detecting mesenchymal stem cells can be improved. Examples of genes exhibiting specifically high expression in mesenchymal stem cells include a mesenchymal stem cell marker gene comprising SEQ ID NOs: 1, 11 and 19 in the sequence listing, and those of genes exhibiting specifically low expression in mesenchymal stem cells include a mesenchymal stem cell marker gene comprising SEQ ID NOs: 2, 4, 6, 7, 9, 10, 12, 14, 15, 17 and 46 in the sequence listing. The mesenchymal stem cell marker gene confirmed in the present invention is also exemplified. Therefore, by combining the expression of one or more genes from each gene group, mesenchymal stem cells can be distinguished with more improved detection accuracy.

In order to detect the expression of a mesenchymal stem cell marker polypeptide in a test cell by a fluorescent antibody method using the antibody of the present invention, and to distinguish and separate mesenchymal stem cells, mesenchymal stem cells can be labeled and separated by known methods. As for the labeling method, for instance, a fluorescent antibody method is exemplified. The labeling of undifferentiated hemopoietic cells by the fluorescent antibody method can be conducted by: fluorescently labeling an antibody that specifically binds to the polypeptide marker for detecting mesenchymal stem cells of the present invention, and labeling mesenchymal stem cells by binding the antibody to mesenchymal stem cells expressing an antigen (direct fluorescent antibody method); or binding the unlabeled specific antibody of the present invention to a mesenchymal stem cell expressing an antigen, and then, by binding a labeled secondary antibody (anti-immunoglobulin antibody), labeling mesenchymal stem cells (indirect fluorescent antibody method). Subsequently, the labeled mesenchymal stem cells are separated and collected.

The probe for detecting mesenchymal stem cell marker genes used for detecting and distinguishing mesenchymal stem cells of the present invention, the microarray or the DNA chip for detecting mesenchymal stem cell marker genes in which the probe is immobilized, and the antibody for detecting mesenchymal stem cells of the present invention can be commercialized as a kit for distinguishing mesenchymal stem cells in which they are included.

The present invention is described below more specifically with reference to Examples, however, the technical scope of the present invention is not limited to these exemplification.

### EXAMPLE 1

### Materials and Methods

(Cells)

Human mesenchymal stem cells and human fibroblasts were cultured at 37° C. in a Dalbecco's modified Eagle's medium (low glucose) (Sigma) supplemented with 10% fetal calf serum and 1 ng/ml bFGF.

(DNA Microarray)

Total RNA was extracted from each cell in one 10 cm-dish by using TRIZOL reagent (Invitrogen), and then mRNA was purified by using micro poly (A) purist (Ambion) to conduct DNA microarray (Incyte Genomics: Kurabo Life Array analysis service; Lot. No. KL01081). The DNA microarray analysis was commissioned to Kurabo.

(Semiquantitative RT-PCR)

Total RNA was extracted from each cell by using TRIZOL reagent (Invitrogen), and then cDNA was synthesized by conducting a reverse transcription reaction at 42° C. for 50 minutes with SuperScript first-strand synthesis system for RT-PCR (Invitrogen) using 1 mg each of total RNA as a template. Each gene was amplified with Advantage 2 PCR enzyme system (Clontech) using the amplified CDNA as a template. The reaction was conducted in 30 cycles, and each cycle included denaturation at 94° C. for 30 seconds and annealing at 68° C. for 1 minute. The primer sequences of each gene used for amplification are tabulated in Table 1. The amplified genes were separated by electrophoresis using 1% agarose gel, and detected by ethidium bromide staining.

TABLE 1

| | Primers (5′ to 3′) | Primers (5′ to 3′) | Probes for |
|---|---|---|---|
| Gene name | for RT-PCR | for real time PCR | real time PCR |
| Adrenomedullin | AGGAATAGTCGCGCAAGCATC | GGTTTCCGTCGCCCTGAT | ACCTGGGTTCGCTCGCCTTCCT |
| | CACGCATTGCACTTTTCCTCTT | GAGCCCACTTATTCCACTTCTTTC | AG |
| Matrix metaroproteinase | CGACTCTAGAAACACAAGAGCAAGA | GATGGACCTGGAGGAAATCTTG | TCATGCTTTTCAACCAGGCCCA |
| 1 | AAGGTTAGCTTACTGTCACACGCTT | CCGCAACACGATGTAAGTTGTACT | GGTATT |
| Tissue factor pathway | GTCGATTCTGCTTTTCC | GGCAACGCCAACAATTTCTAC | CTGGGAGGCTTGCGACGATGC |
| inhibitor 2 | ATGGAATTTTCTTTGGTGCG | CAAACTTTGGGAACTTTTTCTATCCT | |
| Seine (or cysteine) | CCAAACTTTGAAAGCCAAGGTG | TGGGTGGAGAATAACACAAACAA | CTGGTGAAAGATTTGGTATCCC |
| proteinase inhibitor | TGCACTTCAAAGACCAGCAGG | CCAGATAAGTGGCAGCATCAAA | CAAGGG |
| Protein tyrosine | TGGAACCAGAGCGTACGACTGT | GGGAGTTCCTTAATATTCTCAAGTTCTG | TCTCTTGGCCCACTGGTCCCAC |
| kinase 7 | TGGCTTTGCAGCGCTTCTT | GCTGTGTCCTCTATAATCCTGGTCTAG | TTG |
| Collagen, type XV, | ACCAAACCCTATTTCAAGTGCC | CCAGCAACCCACATCAGCTT | TGCCTCCACCAAACCCTATTTC |
| alpha 1 | TAGTTATCCACAAGGCGGACG | ATGCAGAGCAGGCTTCTCATAAT | AAGTGC |
| Sam 68-like | AGGAGAAGGCAAGGATGAAGAA | TCACAACATCAGACAGTACAATCAGTATC | CTCTGTAGACGCCCTTTGCTGT |
| phosphotyrosine protein | TGTTAACTCCTGGAGCTGTGCT | ACGGGCAAGAAGAGTGGACTAA | CCTCG |
| Apolipoprotein D | TGAAGCCACCCCAGTTAACCT | TGAGAAGATCCCAACAACCTTTG | ATGGACGCTGCATCCAGGCCAA |
| | GGTCAACTTCCTTTGTGCGTGGT | TGATCTTTCCGTTTTCCATTAGTG | CTA |
| Major histocompatibility | TGTGTATCCTGCAAAGACCCAG | GGCTGAAGTCCAGAGTGTCCTT | CCTGAAGTAGATGAACAGCCCG |
| complex DR beta | TCCTGTTGGCTGAAGTCCAGA | GCTGGGCCTGCTCTTCCT | GCC |
| Major histocompatibility | TAGCTGTGGACAAAGCCAACCT | GCCCAGGGAAGACCACCTT | TCCGCAAGTTCCACTATCTCCC |
| complex DR alpha | GGCACACACCACGTTCTCTGTA | CAGTCGTAAACGTCCTCAGTTGA | CTTCCT |
| C-type lectin | GCCCCTATGATTGGATTGGTTT | ATCCATTTTCTTTCGGTGTAACATCTA | CTGTTGCTGCACCATCATCGCT |
| | CATCCTTCACTCCCTCTCATGC | CATGAGAGGGAGTGAAGGATGTG | GAG |
| CUG triplet repeat | ATGTTTGTCGGACAGATCCCC | CATGAATGCTTTACAGTTGCAGAA | CTCAGCCACCAGCACCAATGCA |
| RNA-binding protein 2 | ATGTGACAAACGCACAGCCTC | GCGCTGCTCGTGGTAGAGA | AAC |

### EXAMPLE 2

### Detection Results

(DNA Microarray)

As a result of DNA microarray, it was revealed that 63 out of 9400 genes exhibited significantly higher expression, and 141 out of 9400 genes exhibited significantly lower expression in mesenchymal stem cells than in fibroblasts. Significant differences were not observed in other genes. Among 87 genes which exhibited a difference of three-fold or more, 29 genes exhibited higher expression and 58 genes exhibited lower expression in mesenchymal stem cells.

(Semiquantitative RT-PCR)

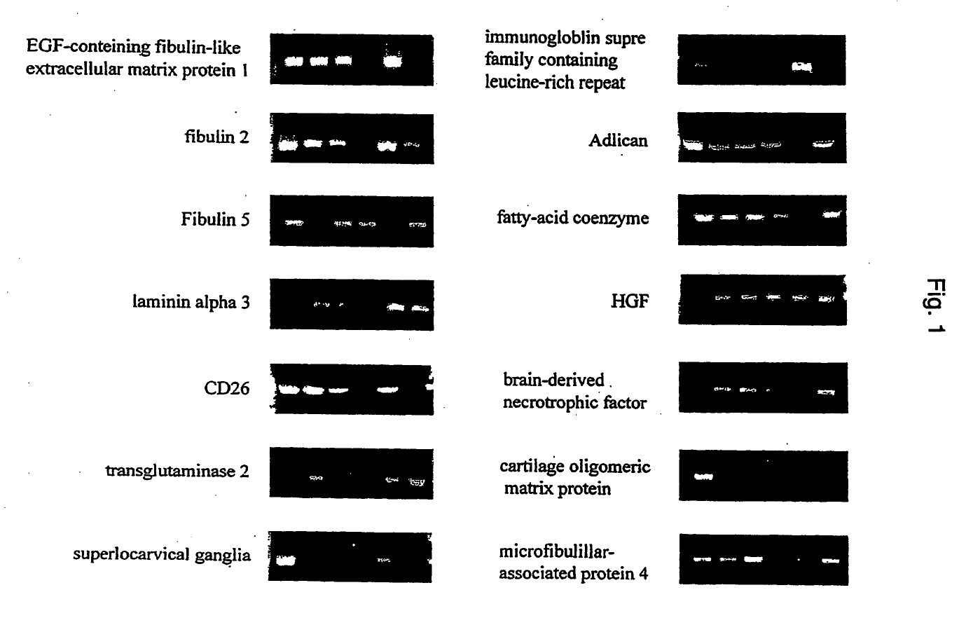

Total RNAs were extracted from mesenchymal stem cells derived from 3 individuals and fibroblasts derived from 3 individuals, and by using them as a template, the expression levels of the above-mentioned 87 genes were examined by semiquantitative RT-PCR. As a result, most genes exhibited no difference in the expression levels. In addition, there were many cases wherein differences in the expression were considered to be caused by individual differences, because differences in the expression levels were observed between mesenchymal stem cells, or fibroblasts, from different individuals (FIG. 1).

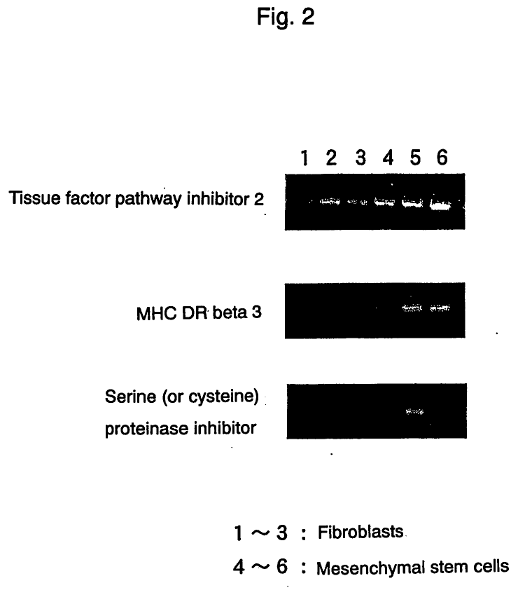

The following 3 genes were confirmed to exhibit high expression in mesenchymal stem cells by RT-PCR: serine (or cysteine) proteinase inhibitor (SEQ ID NO: 1 in the sequence listing), major histocompatibility complex class2 DR beta 3 (SEQ ID NO: 11), and tissue factor pathway inhibitor 2 (SEQ ID NO: 19) (FIG. 2).

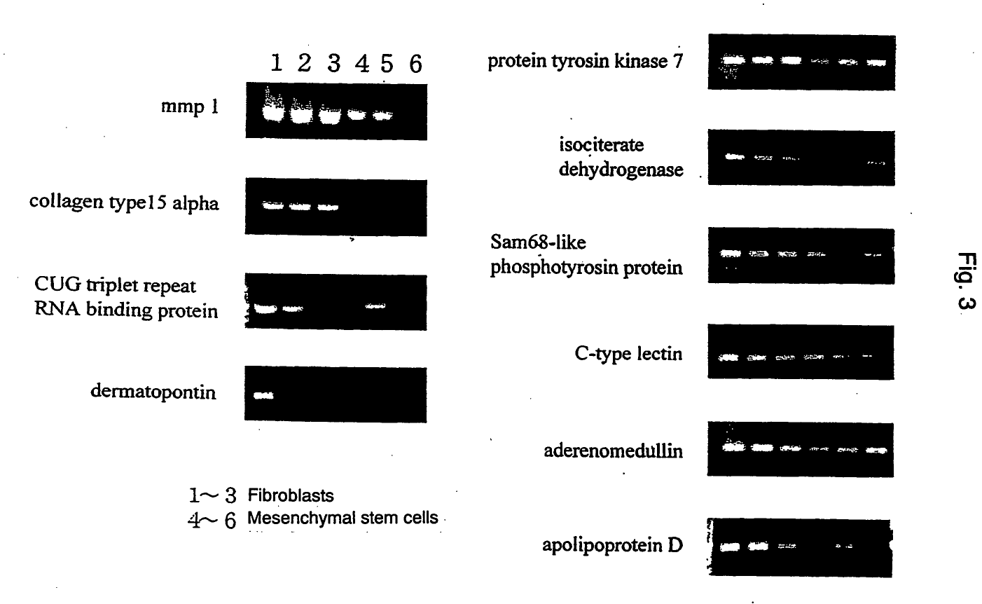

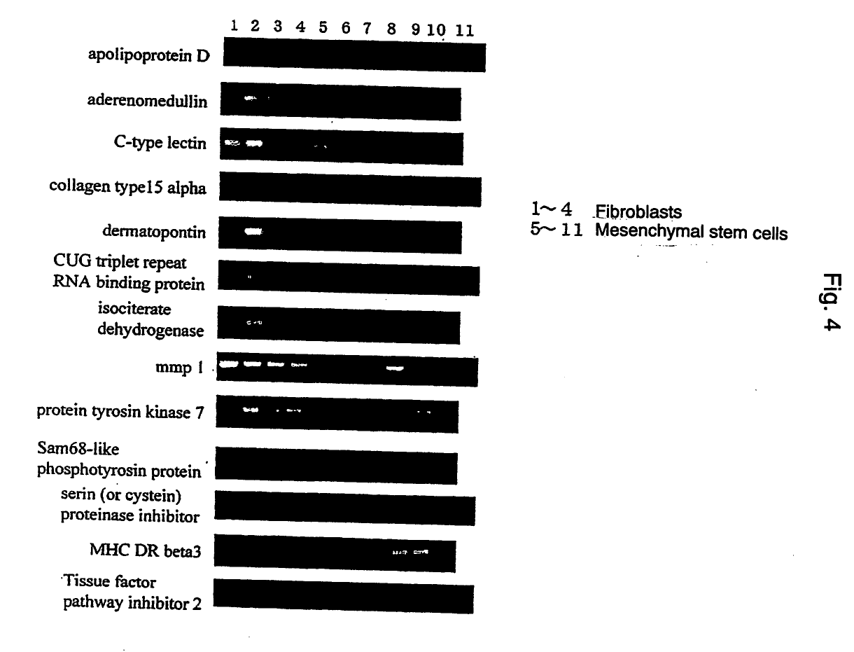

In addition, the following 10 genes were confirmed to exhibit low expression in mesenchymal stem cells by RT-PCR: adrenomedullin (SEQ ID NO: 2), apolipoprotein D (SEQ ID NO: 4), collagenase type XV alpha 1 (SEQ ID NO: 6), CUG triplet repeat RNA binding protein (SEQ ID NO: 7), dermatopontin protein (SEQ ID NO: 9), isocitrate dehydrogenase 2 (SEQ ID NO: 10), tyrosine kinase 7 (SEQ ID NO: 12), Sam68-like phosphotyrosine protein (SEQ ID NO: 14), C-type lectin superfamily member 2 (SEQ ID NO: 15), and matrix metalloprotease 1 (SEQ ID NO: 17) (FIG. 3). Further, with regard to the 13 genes, the expression levels were confirmed by semiquantitative RT-PCR using total RNA extracted from mesenchymal stem cells derived from 7 individuals and fibroblasts derived from 4 individuals as a template (FIG. 4).

### EXAMPLE 3

### Measurement of the Expression of Each Gene in Mesenchymal Stem Cells I

The expressions of the gene marker of the present invention in human mesenchymal stem cells and human fibroblasts were measured in the same manner as in Example 1. The primers for RT-PCR and for real time PCR, and probes for real time PCR used are shown in Table 2.

TABLE 2

| | Primers (5′ to 3′) | Primers (5′ to 3′) | Probes for real |
|---|---|---|---|
| Gene name | for RT-PCR | for real time PCR | time PCR |
| Adrenomedullin | AGGAATAGTCGCGCAAGCATC | GGTTTCCGTCGCCCTGAT | ACCTGGGTTCGCTCGCCTTCCT |
| | CACGCATTGCACTTTTCCTCTT | GAGCCCACTTATTCCACTTCTTTC | AG |
| Matrix metaroproteinase | CGACTCTAGAAACACAAGAGCAAGA | GATGGACCTGGAGGAAATCTTG | TCATGCTTTTCAACCAGGCCCA |
| 1 | AAGGTTAGCTTACTGTCACACGCTT | CCGCAACACGATGTAAGTTGTACT | GGTATT |
| Tissue factor | GTCGATTCTGCTTTTCC | GGCAACGCCAACAATTTCTAC | CTGGGAGGCTTGCGACGATGC |
| pathway inhibitor 2 | ATGGAATTTTCTTTGGTGCG | CAAACTTTGGGAACTTTTTCTATCCT | |
| Serine (or cysteine) | CCAAACTTTGAAAGCCAAGGTG | TGGGTGGAGAATAACACAAACAA | CTGGTGAAAGATTTGGTATCCC |
| proteinase inhibitor | TGCACTTCAAAGACCAGCAGG | CCAGATAAGTGGCAGCATCAAA | CAAGGG |
| Protein tyrosine | TGGAACCAGAGCGTACGACTGT | GGGAGTTCCTTAATATTCTCAAGTTCTG | TCTCTTGGCCCACTGGTCCCAC |
| kinase 7 | TGGCTTTGCAGCGCTTCTT | GCTGTGTCCTCTATAATCCTGGTCTAG | TTG |
| Collagen, type XV, | ACCAAACCCTATTTCAAGTGCC | CCAGCAACCCACATCAGCTT | TGCCTCCACCAAACCCTATTTC |
| alpha 1 | TAGTTATCCACAAGGCGGACG | ATGCAGAGCAGGCTTCTCATAAT | AAGTGC |
| Sam 68-like | AGGAGAAGGCAAGGATGAAGAA | TCACAACATCAGACAGTACAATCAGTATC | CTCTGTAGACGCCCTTTGCTGT |
| phosphotyrosine protein | TGTTAACTCCTGGAGCTGTGCT | ACGGGCAAGAAGAGTGGACTAA | CCTCG |
| Apolipoprotein D | TGAAGCCACCCCAGTTAACCT | TGAGAAGATCCCAACAACCTTTG | ATGGACGCTGCATCCAGGCCAA |
| | GGTCAACTTCCTTTGTGCGTGGT | TGATCTTTCCGTTTTCCATTAGTG | CTA |
| Major histocompatibility | TGTGTATCCTGCAAAGACCCAG | GGCTGAAGTCCAGAGTGTCCTT | CCTGAAGTAGATGAACAGCCCG |
| complex DR beta | TCCTGTTGGCTGAAGTCCAGA | GCTGGGCCTGCTCTTCCT | GCC |
| Major histocompatibility | TAGCTGTGGACAAAGCCAACCT | GCCCAGGGAAGACCACCTT | TCCGCAAGTTCCACTATCTCCC |
| complex DR alpha | GGCACACACCACGTTCTCTGTA | CAGTCGTAAACGTCCTCAGTTGA | CTTCCT |
| C-type lectin | GCCCCTATGATTGGATTGGTTT | ATCCATTTTCTTTCGGTGTAACATCTA | CTGTTGCTGCACCATCATCGCT |
| | CATCCTTCACTCCCTCTCATGC | CATGAGAGGGAGTGAAGGATGTG | GAG |
| CUG triplet repeat | ATGTTTGTCGGACAGATCCCC | CATGAATGCTTTACAGTTGCAGAA | CTCAGCCACCAGCACCAATGCA |
| RNA-binding protein 2 | ATGTGACAAACGCACAGCCTC | GCGCTGCTCGTGGTAGAGA | AAC |

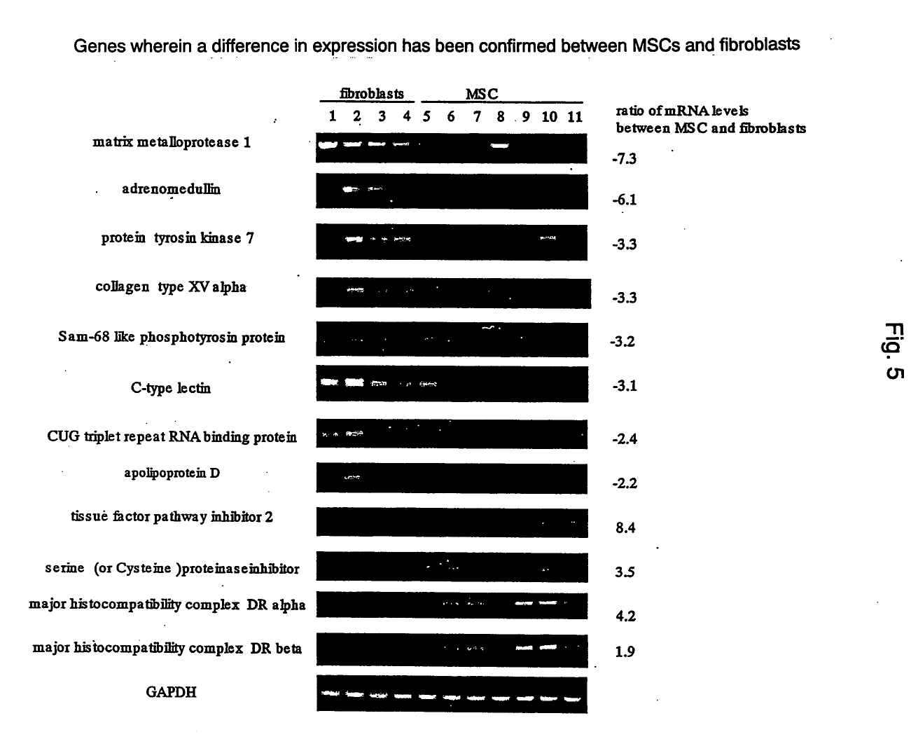

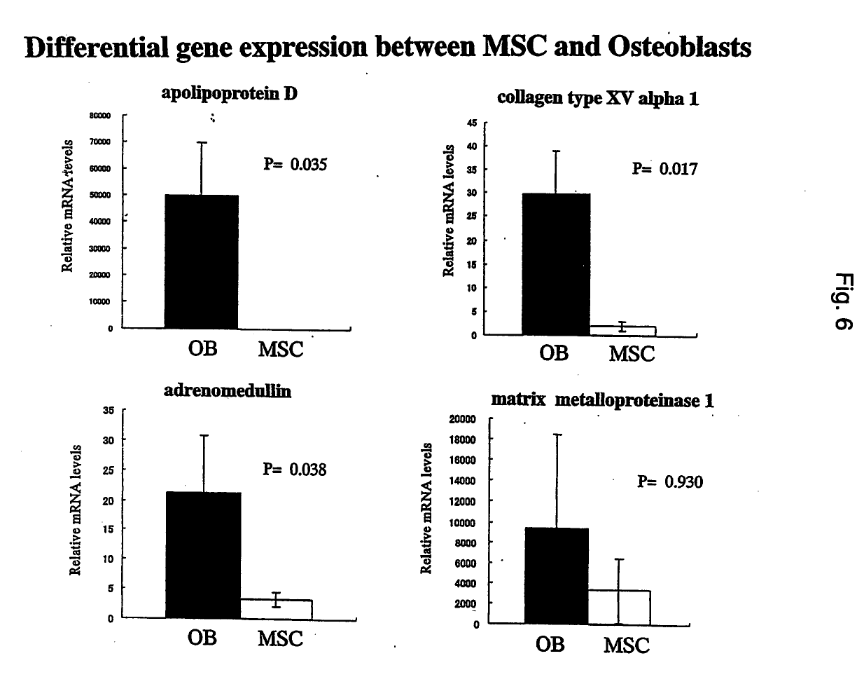

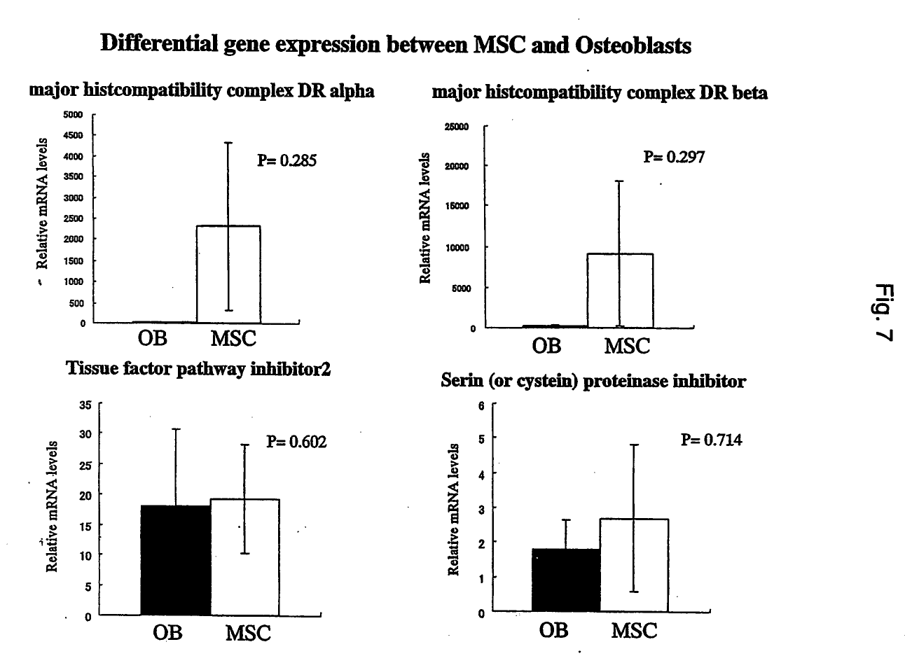

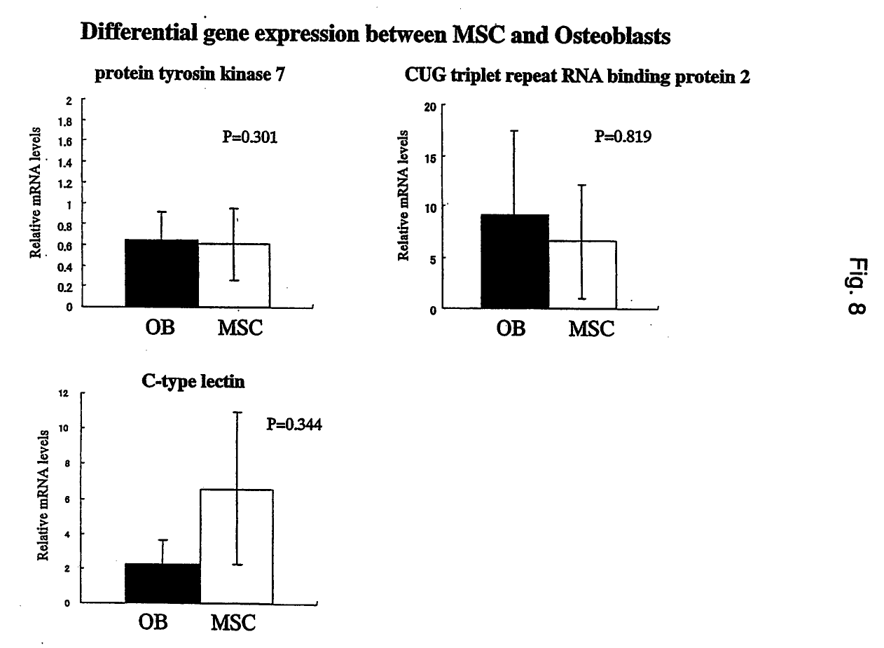

The expression state of mRNA level in mesenchymal stem cells and human fibroblasts are shown in FIG. 5. The relative expression state of mRNA level in mesenchymal stem cells and human fibroblasts are shown in FIGS. 6, 7, and 8.

### EXAMPLE 4

### Measurement of the Expression of Each Gene in Mesenchymal Stem Cells II

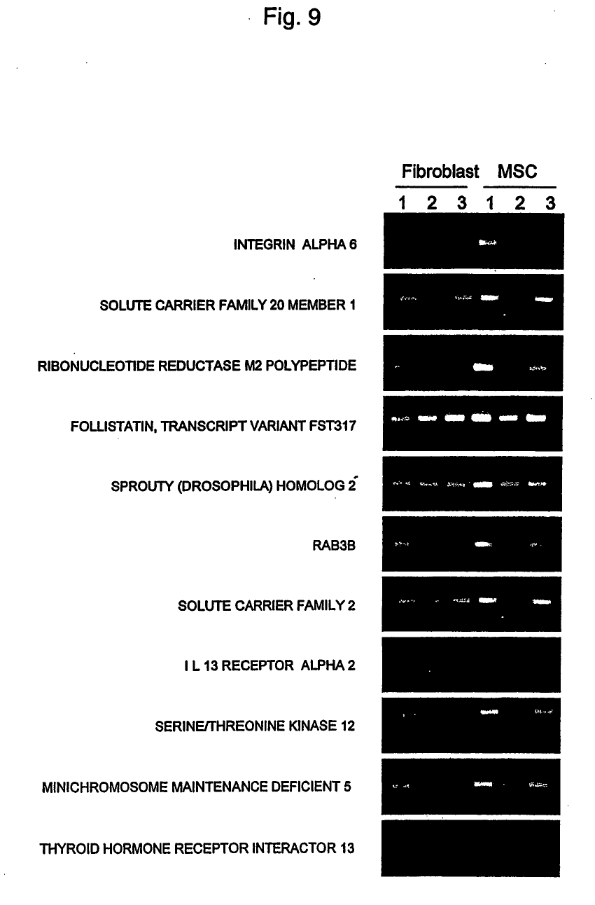

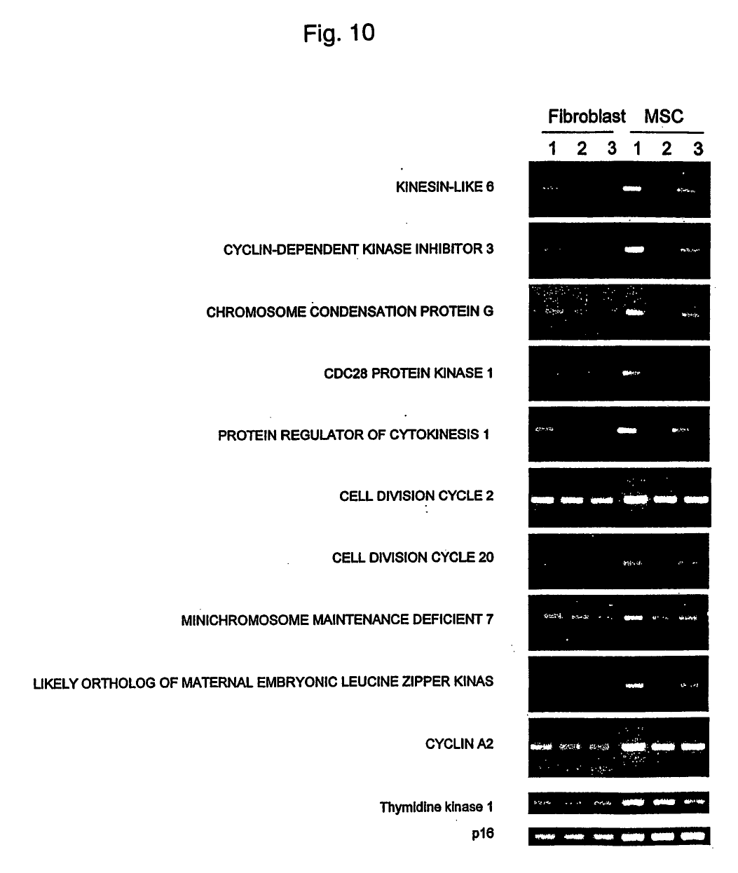

The expressions of each gene marker of the present invention in human mesenchymal stem cells and human fibroblasts were measured in the same manner as in Example 1. The sense and antisense primers for RT-PCR used are shown in SEQ ID NOs: 71 to 116 in the sequence listing. The expression state of genes in mesenchymal stem cells and human fibroblasts are shown in FIGS. 9 and 10. The expression ratio, mesenchymal stem cell/fibroblast, is shown in Table 3.

TABLE 3

| MSC/fibroblast | Gene/name |
|---|---|
| 11.6 | NTEGRIN, ALPHA 6 (ITGA6), MRNA |
| 8.7 | SOLUTE CARRIER FAMILY 20 (PHOSPHATE TRANSPORTER), MEMBER 1 |
| 7.8 | RIBONUCLEOTIDE REDUCTASE M2 POLYPEPTIDE (RRM2), MRNA |
| 7.6 | FOLLISTATIN (FST), TRANSCRIPT VARIANT FST317, MRNA |
| 7.3 | SPROUTY (DROSOPHILA) HOMOLOG 2 (SPRY2), MRNA |
| 6.9 | RAB3B, MEMBER RAS ONCOGENE FAMILY (RAB3B), MRNA |
| 6.5 | SOLUTE CARRIER FAMILY 2 (FACILITATED GLUCOSE TRANSPORTER) |
| 6.1 | INTERLEUKIN 13 RECEPTOR, ALPHA 2 (IL13RA2), MRNA |
| 5.9 | SERINE/THREONINE KINASE 12 (STK12), MRNA |
| 5.7 | MINICHROMOSOME MAINTENANCE DEFICIENT (S. CEREVISIAE) |
| 5.6 | THYROID HORMONE RECEPTOR INTERACTOR 13 (TRIP13), MRNA |
| 5.5 | KINESIN-LIKE 6 (MITOTIC CENTROMERE-ASSOCIATED KINESIN) |
| 5.5 | CYCLIN-DEPENDENT KINASE INHIBITOR 3 |
| 5.4 | CHROMOSOME CONDENSATION PROTEIN G (HCAP-G), MRNA |
| 5.2 | CDC28 PROTEIN KINASE 1 (CKS1), MRNA |
| 5.2 | PROTEIN REGULATOR OF CYTOKINESIS 1 (PRC1), MRNA |
| 5.2 | CELL DIVISION CYCLE 2, G1 TO S AND G2 TO M (CDC2), MRNA |
| 5.2 | CDC20 (CELL DIVISION CYCLE 20, S. CEREVISIAE, HOMOLOG) (CD |
| 5.1 | LIKELY ORTHOLOG OF MATERNAL EMBRYONIC LEUCINE ZIPPER KINAS |
| 5.1 | MINICHROMOSOME MAINTENANCE DEFICIENT (S. CEREVISIAE) 7 |
| 4.9 | YCLIN A2 (CCNA2), MRNA |
| 3.6 | THYMIDINE KINASE 1, SOLUBLE (TK1), MRNA |
| 0.6 | cyclin-dependent kinase inhibitor 2A (melanoma, p16, inhibits CDK4) |
| | (CDKN2A), mRNA |

### EXAMPLE 5

### Measurement of the Expression of Each Gene in Mesenchymal Stem Cells III

The expressions of the gene marker of the present invention in human mesenchymal stem cells and human fibroblasts were measured in the same manner as in Example 1. The expression ratio, mesenchymal stem cell/fibroblast, is shown in Table 4.

TABLE 4

| (Mesenchymal stem cells/fibroblasts) | (Gene name) |
|---|---|
| 8.9 | EGF-containing fibulin-like extracellular matrix protein 1 |
| 8.4 | Human insulin-like growth factor binding protein 5 (IGFBP5) mRNA |
| 7.7 | Homo sapiens clone 24775 mRNA sequence |
| 7 | “proteoglycan 1, secretory granule” |
| 6.8 | insulin-like growth factor binding protein 5 |
| 6.7 | “solute carrier family 21 (organic anion transporter), member 3” |
| 6.5 | “transglutaminase 2 (C polypeptide, proteinglutamine-gamma-glutamyl transferase)” |
| 5.8 | coagulation factor II (thrombin) receptor |
| 5.6 | “plasminogen activator, urokinase” |
| 5.5 | “tissue inhibitor of metalloproteinase 3 (Sorsby fundus dystrophy, pseudoinflammatory)” |
| 5.1 | matrix Gla protein |
| 4.6 | B-cell CLL/lymphoma 1 |
| 4.6 | “CD74 antigen (invariant polypeptide of major histocompatibility complex, class II |
| | antigen-associated)” |
| 4.2 | adducin 3 (gamma) |
| 4.2 | “solute carrier family 16 (monocarboxylic acid transporters), member 4” |
| 3.4 | protein S (alpha) |
| 3.3 | “phosphatidylinositol (4,5) bisphosphate 5-phosphatase homolog; phosphatidylinositol |
| | polyphosphate 5-phosphatase type IV” |
| 3.2 | progesterone membrane binding protein |
| 3.2 | brain-derived neurotrophic factor |
| 3 | apolipoprotein E |
| 38.5 | “GI\|6005949\|REF\|NM_007191.1\| HOMO SAPIENS WNT INHIBITORY FACTOR-1 |
| | (WIF-1), MRNA” |
| 12.7 | “GI\|8922916\|REF\|NM_018349.1\| HOMO SAPIENS HYPOTHETICAL PROTEIN |
| | FLJ11175 (FLJ11175), MRNA” |
| 11.2 | “GI\|10835001\|REF\|NM_001175.1\| HOMO SAPIENS RHO GDP DISSOCIATION |
| | INHIBITOR (GDI) BETA (ARHGDIB), MRNA” |
| 10.3 | “GI\|11055973\|REF\|NM_021614.1\| HOMO SAPIENS POTASSIUM |
| | INTERMEDIATE/SMALL CONDUCTANCE CALCIUM-ACTIVATE” |
| 8.7 | “GI\|5731346\|REF\|NM_004289.3\| HOMO SAPIENS NUCLEAR FACTOR |
| | (ERYTHROID-DERIVED 2)-LIKE 3 (NFE2L3), MRNA” |
| 7.6 | “GI\|11056053\|REF\|NM_021643.1\| HOMO SAPIENS GS3955 PROTEIN (GS3955), |
| | MRNA” |
| 7.5 | “EST10324 ADIPOSE TISSUE, WHITE I HOMO SAPIENS CDNA 3′ END SIMILAR |
| | TO SIMILAR TO GLYCOPROTEIN MUC18,” |
| 6.6 | “GI\|13375818\|REF\|NM_024609.1\| HOMO SAPIENS HYPOTHETICAL PROTEIN |
| | FLJ21841 (FLJ21841), MRNA” |
| 6.5 | “GI\|6005971\|REF\|NM_007150.1\| HOMO SAPIENS ZINC FINGER |
| | PROTEIN 185 (LIM DOMAIN) (ZNF185), MRNA” |
| 6.5 | “GNL\|UG\|HS#S2334464 SC1 = PUTATIVE TRANS-ACTING FACTOR INVOLVED IN |
| | CELL CYCLE CONTROL [HUMAN, MRNA, 24” |
| 6.4 | “GI\|7657368\|REF\|NM_014222.1\| HOMO SAPIENS NADH DEHYDROGENASE |
| | (UBIQUINONE) 1 ALPHA SUBCOMPLEX, 8 (19K” |
| 6.3 | “GI\|8924142\|REF\|NM_018518.1\| HOMO SAPIENS HOMOLOG OF YEAST MCM10; |
| | HYPOTHETICAL PROTEIN PRO2249 (PRO2” |
| 6.2 | “GI\|4502960\|REF\|NM_000094.1\| HOMO SAPIENS COLLAGEN, TYPE VII, ALPHA 1 |
| | (EPIDERMOLYSIS BULLOSA, DYSTRO” |
| 6.2 | “GI\|4502074\|REF\|NM_001144.1\| HOMO SAPIENS AUTOCRINE MOTILITY FACTOR |
| | RECEPTOR (AMFR), MRNA” |
| 6.2 | GNL\|UG\|HS#S1090551 HOMO SAPIENS CLONE 24421 MRNA SEQUENCE |
| | /CDS = UNKNOWN /GB = AF070641 /GI = 3283914 /UG = |
| 6.2 | GNL\|UG\|HS#S1570341 HOMO SAPIENS MRNA; CDNA DKFZP586E1621 (FROM |
| | CLONE DKFZP586E1621) /CDS = UNKNOWN /GB |
| 6.1 | “GI\|11545760\|REF\|NM_022055.1\| HOMO SAPIENS RESERVED (KCNK12), MRNA” |
| 6.0 | “GI\|5902145\|REF\|NM_007019.1\| HOMO SAPIENS UBIQUITIN-CONJUGATING |
| | ENZYME E2C (UBE2C), MRNA” |
| 5.9 | “GNL\|UG\|HS#S1970614 HOMO SAPIENS CDNA FLJ10517 FIS, CLONE |
| | NT2RP2000812 /CDS = (61,918) /GB = AK001379 /G” |
| 5.8 | “GI\|4503052\|REF\|NM_001884.1\| HOMO SAPIENS CARTILAGE LINKING PROTEIN |
| | 1 (CRTL1), MRNA” |
| 5.8 | GNL\|UG\|HS#S1972520 HOMO SAPIENS MRNA; CDNA DKFZP434F2322 (FROM |
| | CLONE DKFZP434F2322) /CDS = UNKNOWN /GB |
| 5.7 | “GNL\|UG\|HS#S3837475 HOMO SAPIENS , CLONE MGC: 9549 IMAGE: 3857382, |
| | MRNA, COMPLETE CDS /CDS = (92,1285) /G” |
| 5.4 | “GI\|14150008\|REF\|NM_032270.1\| HOMO SAPIENS HYPOTHETICAL PROTEIN |
| | DKFZP586J1119 (DKFZP586J1119), MRNA” |
| 5.4 | “GI\|4758439\|REF\|NM_004877.1\| HOMO SAPIENS GLIA MATURATION FACTOR, |
| | GAMMA (GMFG), MRNA” |
| 5.4 | “GNL\|UG\|HS#S2654530 HOMO SAPIENS CDNA: FLJ23095 FIS, CLONE LNG07413 |
| | /CDS = UNKNOWN /GB = AK026748 /GI = 10” |
| 5.3 | “GI\|8922180\|REF\|NM_018410.1\| HOMO SAPIENS HYPOTHETICAL PROTEIN |
| | DKFZP762E1312 (DKFZP762E1312), MRNA” |
| 5.3 | “GI\|5031806\|REF\|NM_005853.1\| HOMO SAPIENS IROQUOIS-CLASS |
| | HOMEODOMAIN PROTEIN (IRX-2A), MRNA” |
| 5.2 | “GI\|8922501\|REF\|NM_018131.1\| HOMO SAPIENS HYPOTHETICAL PROTEIN |
| | FLJ10540 (FLJ10540), MRNA” |
| 5.1 | “GNL\|UG\|HS#S1972896 HOMO SAPIENS MRNA; CDNA DKFZP434C1915 (FROM |
| | CLONE DKFZP434C1915); PARTIAL CDS /C” |
| 5.1 | “GI\|14149622\|REF\|NM_005916.1\| HOMO SAPIENS MINICHROMOSOME |
| | MAINTENANCE DEFICIENT (S. CEREVISIAE) 7 (M” |
| 5.0 | “GI\|4758177\|REF\|NM_004411.1\| HOMO SAPIENS DYNEIN, CYTOPLASMIC, |
| | INTERMEDIATE POLYPEPTIDE 1 (DNCI1), M” |
| 5.0 | HUMAN MRNA FOR PROTEIN GENE PRODUCT (PGP) 9.5. |
| 5.0 | “GI\|5453903\|REF\|NM_006479.1\|HOMO SAPIENS RAD51-INTERACTING PROTEIN |
| | (PIR51), MRNA” |
| 4.9 | “GI\|4502144\|REF\|NM_001168.1\| HOMO SAPIENS BACULOVIRAL IAP |
| | REPEAT-CONTAINING 5 (SURVIVIN) (BIRC5), MR” |
| 4.9 | “GI\|9945386\|REF\|NM_004482.2\| HOMO SAPIENS UDP-N-ACETYL-ALPHA- |
| | D-GALACTOSAMINE:POLYPEPTIDE N-ACETYLGAL” |
| 4.9 | “GI\|14327895\|REF\|NM_031966.1\| HOMO SAPIENS CYCLIN B1 (CCNB1), MRNA” |
| 4.8 | “GI\|6325465\|REF\|NM_004111.3\| HOMO SAPIENS FLAP STRUCTURE-SPECIFIC |
| | ENDONUCLEASE 1 (FEN1), MRNA” |
| 4.8 | “GI\|4507274\|REF\|NM_003600.1\| HOMO SAPIENS SERINE/THREONINE KINASE 15 |
| | (STK15), MRNA” |
| 4.8 | “GI\|6466452\|REF\|NM_002358.2\| HOMO SAPIENS MAD2 (MITOTIC ARREST |
| | DEFICIENT, YEAST, HOMOLOG)-LIKE 1 (MA” |
| 4.7 | “GI\|15421859\|REF\|NM_018454.2\| HOMO SAPIENS NUCLEOLAR PROTEIN ANKT |
| | (ANKT), MRNA” |
| 4.7 | “GI\|7661807\|REF\|NM_014176.1\| HOMO SAPIENS HSPC150 PROTEIN SIMILAR TO |
| | UBIQUITIN-CONJUGATING ENZYME (H” |
| 4.7 | “GI\|9845284\|REF\|NM_019885.1\| HOMO SAPIENS CYTOCHROME P450 RETINOID |
| | METABOLIZING PROTEIN (P450RAI-2),” |
| 4.7 | “GI\|4505372\|REF\|NM_002497.1\| HOMO SAPIENS NIMA (NEVER IN MITOSIS |
| | GENE A)-RELATED KINASE 2 (NEK2), MR” |
| 4.7 | “GI\|4502422\|REF\|NM_001202.1\| HOMO SAPIENS BONE MORPHOGENETIC |
| | PROTEIN 4 (BMP4), MRNA” |
| 4.7 | “GNL\|UG\|HS#S1970775 HOMO SAPIENS CDNA FLJ10674 FIS, CLONE |
| | NT2RP2006436 /CDS = UNKNOWN /GB = AK001536 /GI” |
| 4.7 | “GI\|4585861\|REF\|NM_001809.2\| HOMO SAPIENS CENTROMERE PROTEIN A |
| | (17 KD) (CENPA), MRNA” |
| 4.6 | GNL\|UG\|HS#S1368141 HOMO SAPIENS MRNA; CDNA DKFZP564P116 (FROM |
| | CLONE DKFZP564P116) /CDS = UNKNOWN /GB = A |
| 4.6 | “GI\|4507718\|REF\|NM_003318.1\| HOMO SAPIENS TTK PROTEIN KINASE (TTK), |
| | MRNA” |
| 4.6 | “GI\|4504896\|REF\|NM_002266.1\| HOMO SAPIENS KARYOPHERIN ALPHA 2 (RAG |
| | COHORT 1, IMPORTIN ALPHA 1) (KPNA” |
| 4.6 | “GI\|5729983\|REF\|NM_006596.1\| HOMO SAPIENS POLYMERASE (DNA |
| | DIRECTED), THETA (POLQ), MRNA” |
| 4.5 | “GI\|4758315\|REF\|NM_004454.1\| HOMO SAPIENS ETS VARIANT GENE 5 |
| | (ETS-RELATED MOLECULE) (ETV5), MRNA” |
| 4.5 | “HOMO SAPIENS TRANSFORMING, ACIDIC COILED-COIL CONTAINING |
| | PROTEIN 3 (TAC” |
| 4.5 | “HOMO SAPIENS KINESIN-LIKE 1 (KNSL1), MRNA” |
| 4.5 | “HOMO SAPIENS CENTROMERE PROTEIN E (312 KD) (CENPE), MRNA” |
| 4.5 | “HOMO SAPIENS CDNA, 3′ END /CLONE = IMAGE: 1651289 /CLONE_END = 3′ |
| | /GB = A\|12” |
| 4.4 | “HOMO SAPIENS DISTAL-LESS HOMEO BOX 5 (DLX5), MRNA” |
| 4.4 | “HOMO SAPIENS V-MYB AVIAN MYELOBLASTOSIS VIRAL ONCOGENE |
| | HOMOLOG-LIKE 2” |
| 4.4 | “HOMO SAPIENS SERUM DEPRIVATION RESPONSE (PHOSPHATIDYLSERINE- |
| | BINDING PRO” |
| 4.4 | “HOMO SAPIENS HYPOTHETICAL PROTEIN FLJ10604 (FLJ10604), MRNA” |
| 4.4 | “HOMO SAPIENS RIBONUCLEASE HI, LARGE SUBUNIT (RNASEHI), MRNA |
| 4.4 | “HOMO SAPIENS ANILLIN (DROSOPHILA SCRAPS HOMOLOG), ACTIN BINDING |
| | PROTEIN” |
| 4.4 | “HOMO SAPIENS H4 HISTONE FAMILY, MEMBER G (H4FG), MRNA” |
| 4.3 | “HOMO SAPIENS G PROTEIN-COUPLED RECEPTOR 37 (ENDOTHELIN |
| | RECEPTOR TYPE B-” |
| 4.3 | “HOMO SAPIENS UBIQUITIN CARRIER PROTEIN (E2-EPF), MRNA” |
| 4.3 | “HOMO SAPIENS , SIMILAR TO TUMOR DIFFERENTIALLY EXPRESSED 1, CLONE |
| | IMAGE: 3639252,” |
| 4.2 | “HOMO SAPIENS HYPOTHETICAL PROTEIN MGC2577 (MGC2577), MRNA” |
| 4.2 | “HOMO SAPIENS HYPOTHETICAL PROTEIN FLJ13912 (FLJ13912), MRNA” |
| 4.2 | “HOMO SAPIENS ANNEXIN A3 (ANXA3), MRNA” |
| 4.2 | “HOMO SAPIENS STATHMIN 1/ONCOPROTEIN 18 (STMN1), MRNA” |
| 4.2 | “GNL\|UG\|HS#S3837587 HOMO SAPIENS , SIMILAR TO RIBOSOMAL PROTEIN L39, |
| | CLONE MGC: 20168 IMAGE: 4555759, M” |
| 4.2 | “HOMO SAPIENS POLO (DROSOPHIA)-LIKE KINASE (PLK), MRNA” |

### EXAMPLE 6

### Measurement of the Expression of Each Gene in Mesenchymal Stem Cells VI

The expression of the gene marker of the present invention in human mesenchymal stem cells and human fibroblasts was measured in the same manner as in Example 1. The expression ratio, mesenchymal stem cell/fibroblast, is shown in Table 5. In the table, “-” indicates that the gene exhibits no or extremely low expression in mesenchymal stem cells.

TABLE 5

| (Mesenchymal stem cells/fibroblasts) | (Gene name) |
|---|---|
| −53.576 | “HOMO SAPIENS PROTEASOME (PROSOME, MACROPAIN) 26S SUBUNIT, |
| | ATPASE, 5 (PS” |
| −45.692 | “HOMO SAPIENS EUKARYOTIC TRANSLATION INITIATION FACTOR 4 GAMMA, |
| | 2 (EIF4G” |
| −27.6354 | “HOMO SAPIENS DIHYDROPYRIMIDINASE-LIKE 3 (DPYSL3), MRNA” |
| −20.5446 | “HOMO SAPIENS ADAPTOR-RELATED PROTEIN COMPLEX 1, SIGMA 2 |
| | SUBUNIT (AP1S2)” |
| −19.5809 | “HOMO SAPIENS PLECTIN 1, INTERMEDIATE FILAMENT BINDING PROTEIN, |
| | 500 KD P” |
| −16.9025 | “HOMO SAPIENS HETEROGENEOUS NUCLEAR RIBONUCLEOPROTEIN U |
| | (SCAFFOLD ATTAC” |
| −14.97 | “HOMO SAPIENS HYPOTHETICAL PROTEIN MGC5306 (MGC5306), MRNA” |
| −14.6106 | “HOMO SAPIENS MHC CLASS I POLYPEPTIDE-RELATED SEQUENCE A |
| | (MICA), MRNA” |
| −13.8886 | “HOMO SAPIENS PROTEIN ASSOCIATED WITH PRK1 (AWP1), MRNA” |
| −13.6578 | “HOMO SAPIENS PROGESTERONE RECEPTOR MEMBRANE COMPONENT 2 |
| | (PGRMC2), MRNA” |
| −11.4783 | “HOMO SAPIENS POLYGLUTAMINE BINDING PROTEIN 1 (PQBP1), MRNA” |
| −10.2392 | “HOMO SAPIENS S-ADENOSYLMETHIONINE DECARBOXYLASE 1 (AMD1), |
| | MRNA” |
| −9.9506 | “HOMO SAPIENS INTERFERON-INDUCED, HEPATITIS C-ASSOCIATED |
| | MICROTUBULAR AG” |
| −9.2594 | “HOMO SAPIENS PROTEIN KINASE, AMP-ACTIVATED, GAMMA 1 |
| | NON-CATALYTIC SUBUN” |
| −7.518 | “HOMO SAPIENS PROCOLLAGEN-PROLINE, 2-OXOGLUTARATE |
| | 4-DIOXYGENASE (PROLINE” |
| −6.0365 | “HOMO SAPIENS ENDOTHELIAL DIFFERENTIATION, LYSOPHOSPHATIDIC |
| | ACID G-PROTE” |
| −5.4442 | “HOMO SAPIENS KIAA0127 GENE PRODUCT (KIAA0127), MRNA””” |

### EXAMPLE 7

### Measurement of the Expression of Mesenchymal Stem Cell DR in Human Cells

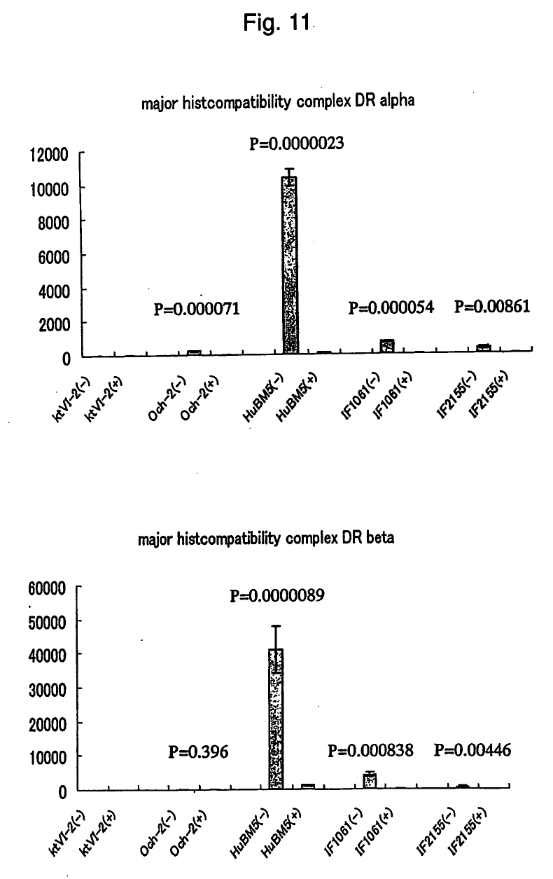

The expression of mesenchymal stem cell DR in human cells was measured with flow cytometry. The results are shown in FIG. 11.

### EXAMPLE 8

### Changes in the Expression of Each Gene in Differentiation Induction of Mesenchymal Stem Cells

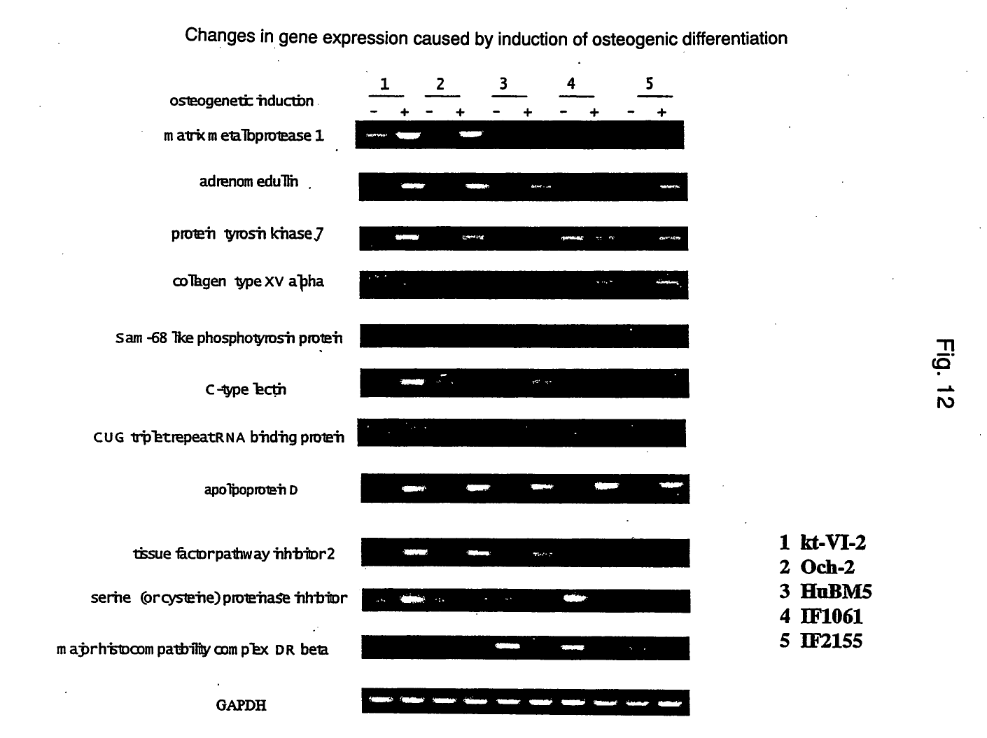

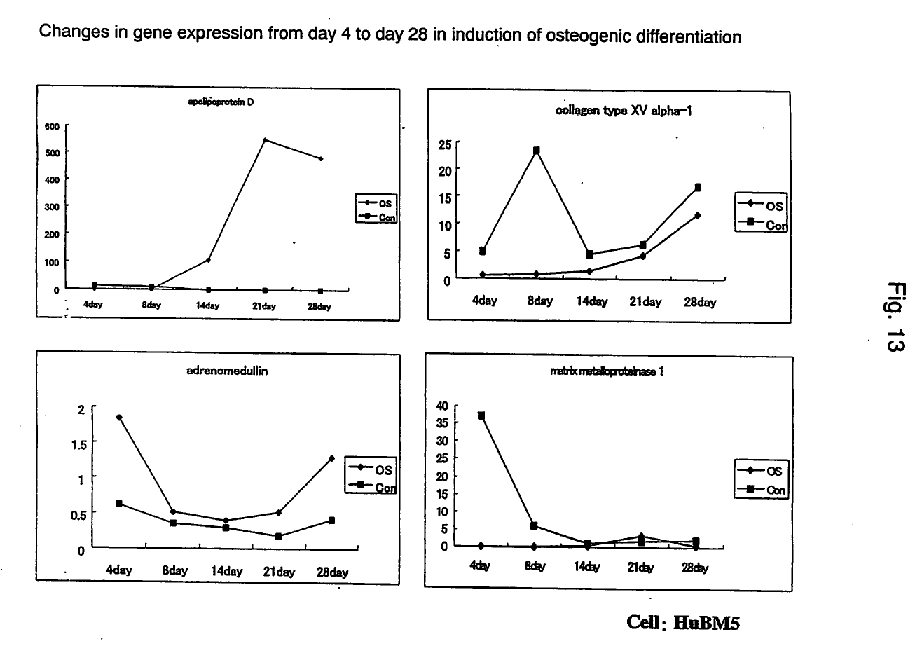

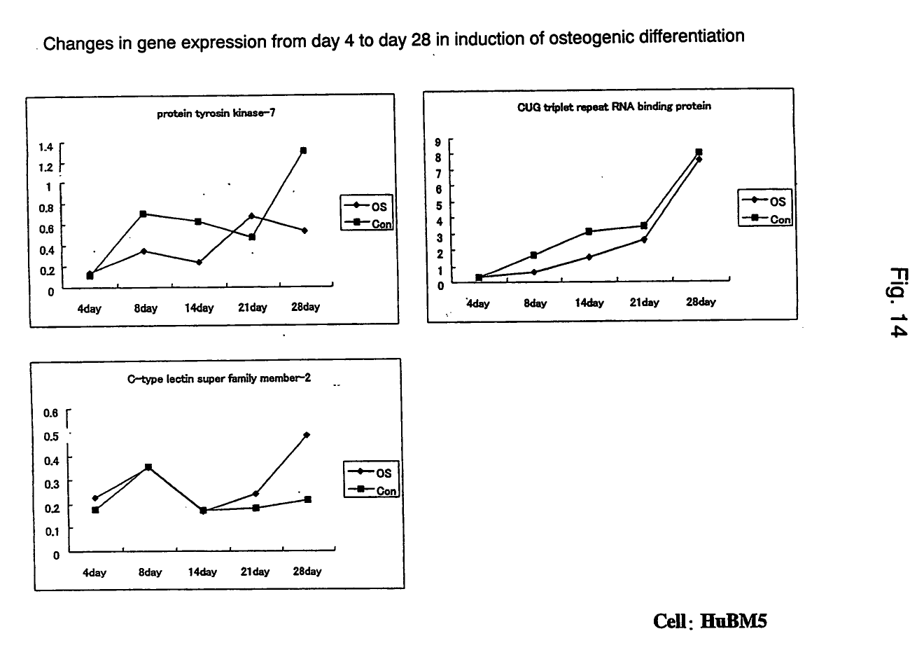

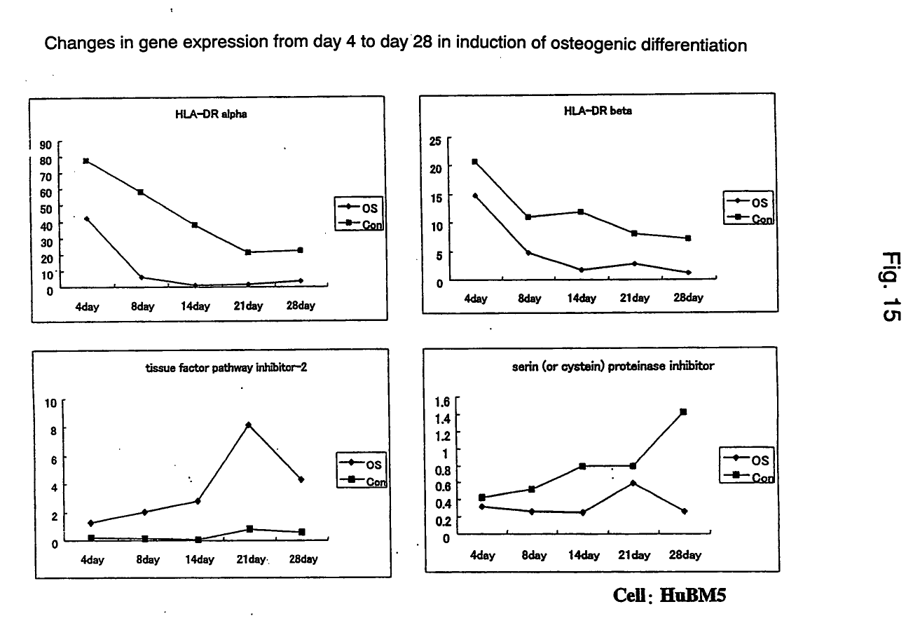

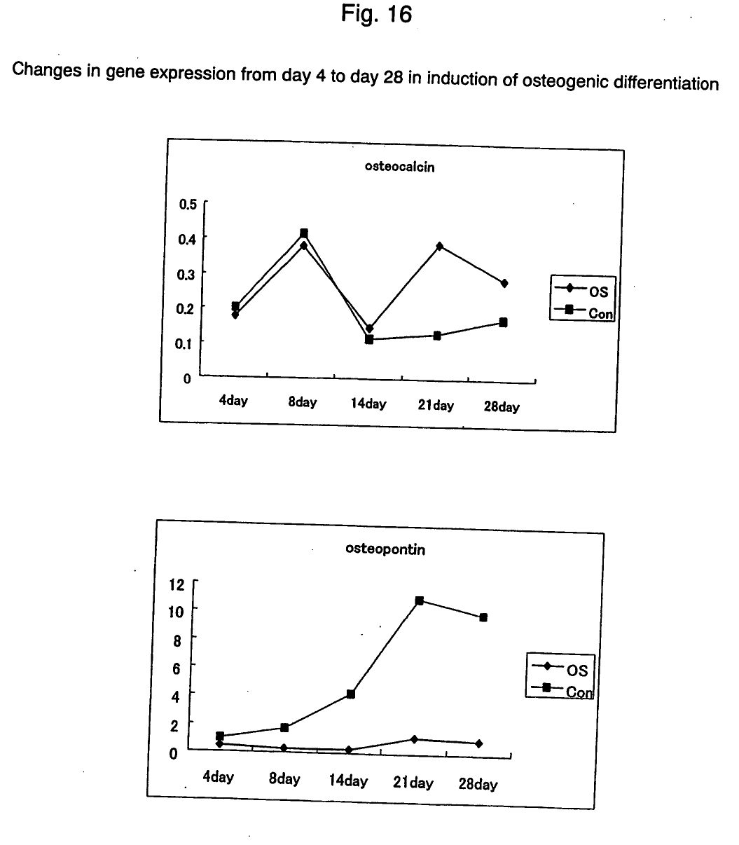

The changes in the expression of each gene in differentiation induction of mesenchymal stem cells were measured by using RT-PCR in the same manner as in Example 1. The expressions of each gene in differentiation induction of osteoblasts are shown in FIGS. 12 to 16.

### INDUSTRIAL APPLICABILITY

Recently, with the development of regenerative medicine, mesenchymal stem cells draw attention as xenograft, homograft and autograft materials for regenerative medicine of many tissues. In order to use mesenchymal stem cells for regenerative medicine of tissues, it is necessary to develop techniques for securing a sufficient amount of mesenchymal stem cells, and to confirm that the cultured cells are mesenchymal stem cells, and a method for detecting and distinguishing the mesenchymal stem cells becomes important. It is also helpful for separating mesenchymal stem cells from a group of many kinds of cells by using antibodies, etc. Marker genes which characterize mesenchymal stem cells are identified in the present invention, and as a result, it becomes possible to detect and distinguish mesenchymal stem cells easily and assuredly, and thereby substantial contribution to the practical use of mesenchymal stem cells in regenerative medicine is expected.

1. A gene marker for detecting a mesenchymal stem cell which is a gene having a base sequence shown in SEQ ID NO: 1, 2, 4, 6, 7, 9, 10, 11, 12, 14, 15, 17, 19 or 46 in the sequence listing.

2. A gene marker for detecting a mesenchymal stem cell which is a gene of INTEGRIN, ALPHA 6 (ITGA6), MRNA (NM_{—}000210); a gene of SOLUTE CARRIER FAMILY 20 (PHOSPHATE TRANSPORTER), MEMBER 1 (NM_{—}005415); a gene of RIBONUCLEOTIDE REDUCTASE M2 POLYPEPTIDE (RRM2), MRNA (NM_{—}001034); a gene of FOLLISTATIN (FST), TRANSCRIPT VARIANT FST317, MRNA (NM_{—}006350); a gene of SPROUTY (DROSOPHILA) HOMOLOG 2 (SPRY2), MRNA (NM_{—}005842); a gene of RAB3B, MEMBER RAS ONCOGENE FAMILY (RAB3B), MRNA (NM_{—}002867); a gene of SOLUTE CARRIER FAMILY 2 (FACILITATED GLUCOSE TRANSPORTER) (NM_{—}006516); a gene of INTERLEUKIN 13 RECEPTOR, ALPHA 2 (IL13RA2), MRNA (NM_{—}000640); a gene of SERINE/THREONINE KINASE 12 (STK12), MRNA (NM_{—}004217); a gene of MINICHROMOSOME MAINTENANCE DEFICIENT (S. CEREVISIAE) 5 (CE) (NM_{—}006739); a gene of THYROID HORMONE RECEPTOR INTERACTOR 13 (TRIP13), MRNA (NM_{—}004237); a gene of KINESIN-LIKE 6 (MITOTIC CENTROMERE-ASSOCIATED KINESIN) (K) (NM_{—}006845); a gene of CYCLIN-DEPENDENT KINASE INHIBITOR 3 (CDK2-ASSOCIATED DUAL (NM_{—}005192); a gene of CHROMOSOME CONDENSATION PROTEIN G (HCAP-G), MRNA (NM_{—}022346); a gene of CDC28 PROTEIN KINASE 1 (CKS1), MRNA(NM_{—}001826); a gene of PROTEIN REGULATOR OF CYTOKINESIS 1 (PRC1), MRNA (NM_{—}003981); a gene of CELL DIVISION CYCLE 2, G1 TO S AND G2 TO M (CDC2), MRNA (NM_{—}001786); a gene of CDC20 (CELL DIVISION CYCLE 20, S. CEREVISIAE, HOMOLOG) (CD) (NM_{—}001255); a gene of LIKELY ORTHOLOG OF MATERNAL EMBRYONIC LEUCINE ZIPPER KINAS (NM_{—}014791); a gene of MINICHROMOSOME MAINTENANCE DEFICIENT (S. CEREVISIAE) 7 (M) (NM_{—}005916); a gene of CYCLIN A2 (CCNA2), MRNA (NM_{—}001237); a gene of THYMIDINE KINASE 1, SOLUBLE (TK1), MRNA (NM_{—}003258); or a gene of cyclin-dependent kinase inhibitor 2A (melanoma, p16, inhibits CDK4) (CDKN2A), mRNA (NM_{—}000077).

3. A gene marker for detecting a mesenchymal stem cell which is a gene of EGF-containing fibulin-like extracellular matrix protein 1 (NM_{—}018894); a gene of Human insulin-like growth factor binding protein 5 (IGFBP5) mRNA (AU132011); a gene of Homo sapiens clone 24775 MRNA sequence (AA402981); proteoglycan 1, secretory granule (AV734015); a gene of insulin-like growth factor binding protein 5 (AA374325); a gene of solute carrier family 21 (organic anion transporter), member 3 (AF085224); a gene of transglutaminase 2 (C polypeptide, protein-glutamine-gamma-glutamyl transferase) (AL552373); a gene of coagulation factor II (thrombin) receptor (M62424); a gene of plasminogen activator, urokinase (NM_{—}002658); a gene of tissue inhibitor of metalloproteinase 3 (Sorsby fundus dystrophy, pseudoinflammatory) (W96324); a gene of matrix Gla protein (BF668572); a gene of B-cell CLL/lymphoma 1 (Z23022); a gene of CD74 antigen (invariant polypeptide of major histocompatibility complex, class II antigen-associated) (BG333618); a gene of adducin 3 (gamma) (AL135243); a gene of solute carrier family 16 (monocarboxylic acid transporters), member 4 (NM_{—}004696); a gene of protein S (alpha) (NM_{—}000313); a gene of phosphatidylinositol (4,5) bisphosphate 5-phosphatase homolog; phosphatidylinositol polyphosphate 5-phosphatase type IV (AF187891); a gene of progesterone membrane binding protein (BE858855); a gene of brain-derived neurotrophic factor (X60201); or a gene of apolipoprotein E (BF967316).

4. A gene marker for detecting a mesenchymal stem cell which is a gene of HOMO SAPIENS WNT INHIBITORY FACTOR-1 (WIF-1), MRNA(NM 007191); a gene of HOMO SAPIENS HYPOTHETICAL PROTEIN FLJ11175 (FLJ11175), MRNA (NM_{—}018349); a gene of HOMO SAPIENS RHO GDP DISSOCIATION INHIBITOR (GDI) BETA (ARHGDIB), MRNA (NM_{—}001175); a gene of HOMO SAPIENS POTASSIUM INTERMEDIATE/SMALL CONDUCTANCE CALCIUM-ACTIVATE (NM_{—}021614); a gene of HOMO SAPIENS NUCLEAR FACTOR (ERYTHROID-DERIVED 2)-LIKE 3 (NFE2L3), MRNA (NM_{—}004289); a gene of HOMO SAPIENS GS3955 PROTEIN (GS3955), MRNA (NM 021643); a gene of HOMO SAPIENS CDNA 3′ END SIMILAR TO SIMILAR TO GLYCOPROTEIN MUC18 (AA302605); a gene of HOMO SAPIENS HYPOTHETICAL PROTEIN FLJ21841 (FLJ21841), MRNA (NM_{—}024609); a gene of HOMO SAPIENS ZINC FINGER PROTEIN 185 (LIM DOMAIN) (ZNF185), MRNA (NM_{—}007150); a gene of SC1=PUTATIVE TRANS-ACTING FACTOR INVOLVED IN CELL CYCLE CONTROL [HUMAN, MRNA, 24] (S53374); a gene of HOMO SAPIENS NADH DEHYDROGENASE (UBIQUINONE) 1 ALPHA SUBCOMPLEX, 8 (19K) (NM_{—}014222); a gene of HOMO SAPIENS HOMOLOG OF YEAST MCM10; HYPOTHETICAL PROTEIN PRO2249 (PRO2) (NM_{—}018518); a gene of HOMO SAPIENS COLLAGEN, TYPE VII, ALPHA 1 (EPIDERMOLYSIS BULLOSA, DYSTRO (NM_{—}000094); a gene of HOMO SAPIENS AUTOCRINE MOTILITY FACTOR RECEPTOR (AMFR), MRNA (NM_{—}001144); a gene of HOMO SAPIENS CLONE 24421 MRNA SEQUENCE /CDS=UNKNOWN/GB=AF070641/GI=3283914/UG=(AF070641); a gene of HOMO SAPIENS MRNA; a gene of CDNA DKFZP586E1621 (FROM CLONE DKFZP586E1621)/CDS=UNKNOWN/GB (AL080235).

5. A gene marker for detecting a mesenchymal stem cell which is a gene of HOMO SAPIENS RESERVED (KCNK12), MRNA (NM_{—}022055); a gene of HOMO SAPIENS UBIQUITIN-CONJUGATING ENZYME E2C (UBE2C), MRNA (NM_{—}007019); a gene of HOMO SAPIENS CDNA FLJ10517 FIS, CLONE NT2RP2000812/CDS=(61,918)/GB=AK001379/G (AK001379); a gene of HOMO SAPIENS CARTILAGE LINKING PROTEIN 1 (CRTL1), MRNA (NM_{—}001884); a gene of HOMO SAPIENS MRNA; CDNA DKFZP434F2322 (FROM CLONE DKFZP434F2322)/CDS=UNKNOWN/GB (AL133105); a gene of HOMO SAPIENS, CLONE MGC:9549 IMAGE:3857382, MRNA, COMPLETE CDS/CDS=(92,1285)/G (BC012453); a gene of HOMO SAPIENS HYPOTHETICAL PROTEIN DKFZP586J1119 (DKFZP586J1119), MRNA (NM_{—}032270); a gene of HOMO SAPIENS GLIA MATURATION FACTOR, GAMMA (GMFG), MRNA (NM_{—}004877); a gene of HOMO SAPIENS CDNA: FLJ23095 FIS, CLONE LNG07413/CDS=UNKNOWN/GB=AK026748/GI=10 (AK026748); a gene of HOMO SAPIENS HYPOTHETICAL PROTEIN DKFZP762E1312 (DKFZP762E1312), MRNA (NM_{—}018410); a gene of HOMO SAPIENS IROQUOIS-CLASS HOMEODOMAIN PROTEIN (IRX-2A), MRNA (NM_{—}005853); or a gene of HOMO SAPIENS HYPOTHETICAL PROTEIN FLJ10540 (FLJ10540), MRNA (NM_{—}018131).

6. A gene marker for detecting a mesenchymal stem cell which is a gene of HOMO SAPIENS MRNA; CDNA DKFZP434C1915 (FROM CLONE DKFZP434C1915); PARTIAL CDS/C (AL137698); a gene of HOMO SAPIENS MINICHROMOSOME MAINTENANCE DEFICIENT (S. CEREVISIAE) 7 (M) (NM_{—}005916); a gene of HOMO SAPIENS DYNEIN, CYTOPLASMIC, INTERMEDIATE POLYPEPTIDE 1 (DNCI1), M (NM_{—}004411); a gene of HUMAN MRNA FOR PROTEIN GENE PRODUCT (PGP) 9.5. (X04741); a gene of HOMO SAPIENS RAD51-INTERACTING PROTEIN (PIR51), MRNA (NM_{—}006479); a gene of HOMO SAPIENS BACULOVIRAL IAP REPEAT-CONTAINING 5 (SURVIVIN) (BIRC5), MR (NM_{—}001168); a gene of HOMO SAPIENS UDP-N-ACETYL-ALPHA-D-GALACTOSAMINE:POLYPEPTIDE N-ACETYLGAL (NM_{—}004482); a gene of HOMO SAPIENS CYCLIN B1 (CCNB1), MRNA (NM_{—}031966); a gene of HOMO SAPIENS FLAP STRUCTURE-SPECIFIC ENDONUCLEASE 1 (FEN1), MRNA (NM_{—}004111); a gene of HOMO SAPIENS SERINE/THREONINE KINASE 15 (STK15), MRNA (NM_{—}003600); a gene of HOMO SAPIENS MAD2 (MITOTIC ARREST DEFICIENT, YEAST, HOMOLOG)-LIKE 1 (MA) (NM_{—}002358); a gene of HOMO SAPIENS NUCLEOLAR PROTEIN ANKT (ANKT), MRNA (NM_{—}018454); a gene of HOMO SAPIENS HSPC150 PROTEIN SIMILAR TO UBIQUITIN-CONJUGATING ENZYME (H) (NM_{—}014176); a gene of HOMO SAPIENS CYTOCHROME P450 RETINOID METABOLIZING PROTEIN (P450RAI-2), (NM_{—}019885); a gene of HOMO SAPIENS NIMA (NEVER IN MITOSIS GENE A)-RELATED KINASE 2 (NEK2), MR (NM_{—}002497); a gene of HOMO SAPIENS BONE MORPHOGENETIC PROTEIN 4 (BMP4), MRNA (NM_{—}001202); a gene of HOMO SAPIENS CDNA FLJ10674 FIS, CLONE NT2RP2006436/CDS=UNKNOWN/GB=AK001536/GI (AK001536); or a gene of HOMO SAPIENS CENTROMERE PROTEIN A (17 KD) (CENPA), MRNA (NM_{—}001809).

7. A gene marker for detecting a mesenchymal stem cell which is a gene of HOMO SAPIENS MRNA; CDNA DKFZP564P116 (FROM CLONE DKFZP564P116)/CDS=UNKNOWN/GB=A (AL049338); a gene of HOMO SAPIENS TTK PROTEIN KINASE (TTK), MRNA (NM_{—}003318); a gene of HOMO SAPIENS KARYOPHERIN ALPHA 2 (RAG COHORT 1, IMPORTIN ALPHA 1) (KPNA (NM_{—}002266); a gene of HOMO SAPIENS POLYMERASE (DNA DIRECTED), THETA (POLQ), MRNA (NM_{—}006596); a gene of HOMO SAPIENS ETS VARIANT GENE 5 (ETS-RELATED MOLECULE) (ETV5), MRNA (NM_{—}004454); a gene of HOMO SAPIENS TRANSFORMING, ACIDIC COILED-COIL CONTAINING PROTEIN 3 (TAC) (NM_{—}006342); a gene of HOMO SAPIENS KINESIN-LIKE 1 (KNSL1), MRNA (NM_{—}004523); a gene of HOMO SAPIENS CENTROMERE PROTEIN E (312 KD) (CENPE), MRNA (NM_{—}001813); a gene of HOMO SAPIENS CDNA, 3′ END/CLONE=IMAGE:1651289/CLONE_END=3′/GB=AI12 (AI123815); a gene of HOMO SAPIENS DISTAL-LESS HOMEO BOX 5 (DLX5), MRNA (NM_{—}005221); a gene of HOMO SAPIENS V-MYB AVIAN MYELOBLASTOSIS VIRAL ONCOGENE HOMOLOG-LIKE 2 (NM_{—}002466); or a gene of HOMO SAPIENS SERUM DEPRIVATION RESPONSE (PHOSPHATIDYLSERINE-BINDING PRO (NM_{—}004657).

8. A gene marker for detecting a mesenchymal stem cell which is a gene of HOMO SAPIENS HYPOTHETICAL PROTEIN FLJ10604 (FLJ10604), MRNA (NM_{—}018154); a gene of HOMO SAPIENS RIBONUCLEASE HI, LARGE SUBUNIT (RNASEHI), MRNA (NM_{—}006397); a gene of HOMO SAPIENS ANILLIN (DROSOPHILA SCRAPS HOMOLOG), ACTIN BINDING PROTEIN (NM_{—}018685); a gene of HOMO SAPIENS H4 HISTONE FAMILY, MEMBER G (H₄FG), MRNA (NM_{—}003542); a gene of HOMO SAPIENS G PROTEIN-COUPLED RECEPTOR 37 (ENDOTHELIN RECEPTOR TYPE B-(NM_{—}005302) HOMO SAPIENS UBIQUITIN CARRIER PROTEIN (E2-EPF), MRNA (NM_{—}014501); a gene of HOMO SAPIENS, SIMILAR TO TUMOR DIFFERENTIALLY EXPRESSED 1, CLONE IMAGE:3639252, (BC007375); a gene of HOMO SAPIENS HYPOTHETICAL PROTEIN MGC2577 (MGC2577), MRNA (NM_{—}031299); a gene of HOMO SAPIENS HYPOTHETICAL PROTEIN FLJ13912 (FLJ13912), MRNA (NM_{—}022770); a gene of HOMO SAPIENS ANNEXIN A3 (ANXA3), MRNA (NM_{—}005139); a gene of HOMO SAPIENS STATHMIN 1/ONCOPROTEIN 18 (STMN1), MRNA (NM_{—}005563) HOMO SAPIENS, SIMILAR TO RIBOSOMAL PROTEIN L39, CLONE MGC:20168 IMAGE:4555759, M (BC012328); or a gene of HOMO SAPIENS POLO (DROSOPHIA)-LIKE KINASE (PLK), MRNA (NM 005030).

9. A gene marker for detecting a mesenchymal stem cell which is a gene of HOMO SAPIENS PROTEASOME (PROSOME, MACROPAIN) 26S SUBUNIT, ATPASE, 5 (PS) (NM_{—}002805); a gene of HOMO SAPIENS EUKARYOTIC TRANSLATION INITIATION FACTOR 4 GAMMA, 2 (EIF4G) (NM_{—}001418); a gene of HOMO SAPIENS DIHYDROPYRIMIDINASE-LIKE 3 (DPYSL3), MRNA) (NM_{—}001387); a gene of HOMO SAPIENS ADAPTOR-RELATED PROTEIN COMPLEX 1, SIGMA 2 SUBUNIT (AP1S2) (NM_{—}003916); a gene of HOMO SAPIENS PLECTIN 1, INTERMEDIATE FILAMENT BINDING PROTEIN, 500 KD (P) (NM_{—}000445); a gene of HOMO SAPIENS HETEROGENEOUS NUCLEAR RIBONUCLEOPROTEIN U (SCAFFOLD ATTAC (NM_{—}031844); a gene of HOMO SAPIENS HYPOTHETICAL PROTEIN MGC5306 (MGC5306), MRNA (NM_{—}024116); a gene of HOMO SAPIENS MHC CLASS I POLYPEPTIDE-RELATED SEQUENCE A (MICA), MRNA (NM_{—}000247); a gene of HOMO SAPIENS PROTEIN ASSOCIATED WITH PRK1 (AWP1), MRNA (NM_{—}019006); a gene of HOMO SAPIENS PROGESTERONE RECEPTOR MEMBRANE COMPONENT 2 (PGRMC2), MRNA (NM_{—}006320); a gene of HOMO SAPIENS POLYGLUTAMINE BINDING PROTEIN 1 (PQBP1), MRNA (NM_{—}005710); a gene of HOMO SAPIENS S-ADENOSYLMETHIONINE DECARBOXYLASE 1 (AMD1), MRNA (NM_{—}001634); HOMO SAPIENS INTERFERON-INDUCED, HEPATITIS C-ASSOCIATED MICROTUBULAR AG (NM_{—}006417); a gene of HOMO SAPIENS PROTEIN KINASE, AMP-ACTIVATED, GAMMA 1 NON-CATALYTIC SUBUN (NM_{—}002733); HOMO SAPIENS PROCOLLAGEN-PROLINE, 2-OXOGLUTARATE 4-DIOXYGENASE (PROLINE (NM_{—}004199); a gene of HOMO SAPIENS ENDOTHELIAL DIFFERENTIATION, LYSOPHOSPHATIDIC ACID G-PROTE (NM_{—}012152); or a gene of HOMO SAPIENS KIAA0127 GENE PRODUCT (KIAA0127), MRNA (NM_{—}014755).

10. A probe for detecting a mesenchymal stem cell marker gene having a DNA sequence which hybridizes with the marker gene for detecting a mesenchymal stem cell according to any one of claims 1 to 9 under a stringent condition.

11. The probe for detecting a mesenchymal stem cell marker gene according to claim 10, which comprises whole or part of an antisense strand of the base sequence according to any one of claims 1 to 9.

12. A microarray or a DNA chip for detecting a mesenchymal stem cell marker gene, wherein at least one of the DNA according to claim 10 or 11 is immobilized.

13. The microarray or the DNA chip for detecting a mesenchymal stem cell marker gene according to claim 12, wherein a probe for detecting two or more genes in a group of genes having a base sequence shown in SEQ ID NO: 1, 2, 4, 6, 7, 9, 10, 11, 12, 14, 15, 17, 19 or 46 in the sequence listing and a group of the genes according to any one of claims 2 to 9 is immobilized.

14. A polypeptide marker for detecting a mesenchymal stem cell which is a polypeptide having an amino acid sequence shown in SEQ ID NO: 3, 5, 8, 13, 16 or 18 in the sequence listing.

15. An antibody which is induced by using the polypeptide according to claim 14 and which specifically binds to the polypeptide.

16. The antibody according to claim 15, which is a monoclonal antibody.

17. The antibody according to claim 15, which is a polyclonal antibody.

18. A method for distinguishing a mesenchymal stem cell wherein expression of the mesenchymal stem cell marker gene according to any one of claims 1 to 9 in a test cell is detected.

19. The method for distinguishing a mesenchymal stem cell according to claim 18, wherein the expression of the mesenchymal stem cell marker gene is detected by using Northern blotting.

20. The method for distinguishing a mesenchymal stem cell according to claim 18, wherein the expression of the mesenchymal stem cell marker gene is detected by using the probe for detecting a mesenchymal stem cell marker gene according to claim 10 or 11.

21. The method for distinguishing a mesenchymal stem cell according to claim 18, wherein the expression of the mesenchymal stem cell marker gene is detected by using the microarray or the DNA chip for detecting a mesenchymal stem cell marker gene according to claim 12 or 13.

22. A method for distinguishing a mesenchymal stem cell, wherein the detection of the expression of the mesenchymal stem cell marker gene according to any one of claims 18 to 21 comprises use of quantitative or semiquantitative PCR.

23. The method for distinguishing a mesenchymal stem cell according to claim 22, wherein the use of quantitative or semiquantitative PCR is RT-PCR method.

24. An RT-PCR primer for amplifying a mesenchymal stem cell marker gene which comprises a sense primer having a base sequence shown in SEQ ID NO: 20 in the sequence listing, and an antisense primer having a base sequence shown in SEQ ID NO: 21 in the sequence listing; a sense primer having a base sequence shown in SEQ ID NO: 22 in the sequence listing, and an antisense primer having a base sequence shown in SEQ ID NO: 23 in the sequence listing; a sense primer having a base sequence shown in SEQ ID NO: 24 in the sequence listing, and an antisense primer having a base sequence shown in SEQ ID NO: 25 in the sequence listing; a sense primer having a base sequence shown in SEQ ID NO: 26 in the sequence listing, and an antisense primer having a base sequence shown in SEQ ID NO: 27 in the sequence listing; a sense primer having a base sequence shown in SEQ ID NO: 28 in the sequence listing, and an antisense primer having a base sequence shown in SEQ ID NO: 29 in the sequence listing; a sense primer having a base sequence shown in SEQ ID NO: 30 in the sequence listing, and an antisense primer having a base sequence shown in SEQ ID NO: 31 in the sequence listing; a sense primer having a base sequence shown in SEQ ID NO: 32 in the sequence listing, and an antisense primer having a base sequence shown in SEQ ID NO: 33 in the sequence listing; a sense primer having a base sequence shown in SEQ ID NO: 34 in the sequence listing, and an antisense primer having a base sequence shown in SEQ ID NO: 35 in the sequence listing; a sense primer having a base sequence shown in SEQ ID NO: 36 in the sequence listing, and an antisense primer having a base sequence shown in SEQ ID NO: 37 in the sequence listing; a sense primer having a base sequence shown in SEQ ID NO: 38 in the sequence listing, and an antisense primer having a base sequence shown in SEQ ID NO: 39 in the sequence listing; a sense primer having a base sequence shown in SEQ ID NO: 40 in the sequence listing, and an antisense primer having a base sequence shown in SEQ ID NO: 41 in the sequence listing; a sense primer having a base sequence shown in SEQ ID NO: 42 in the sequence listing, and an antisense primer having a base sequence shown in SEQ ID NO: 43 in the sequence listing; or a sense primer having a base sequence shown in SEQ ID NO: 44 in the sequence listing, and an antisense primer having abase sequence shown in SEQ ID NO: 45 in the sequence listing.

25. A real time PCR primer for amplifying a mesenchymal stem cell marker gene which comprises a sense primer having a base sequence shown in SEQ ID NO: 47 in the sequence listing, and an antisense primer having a base sequence shown in SEQ ID NO: 48 in the sequence listing; a sense primer having a base sequence shown in SEQ ID NO: 49 in the sequence listing, and an antisense primer having a base sequence shown in SEQ ID NO: 50 in the sequence listing; a sense primer having a base sequence shown in SEQ ID NO: 51 in the sequence listing, and an antisense primer having a base sequence shown in SEQ ID NO: 52 in the sequence listing; a sense primer having a base sequence shown in SEQ ID NO: 53 in the sequence listing, and an antisense primer having a base sequence shown in SEQ ID NO: 54 in the sequence listing; a sense primer having a base sequence shown in SEQ ID NO: 55 in the sequence listing, and an antisense primer having a base sequence shown in SEQ ID NO: 56 in the sequence listing; a sense primer having a base sequence shown in SEQ ID NO: 57 in the sequence listing, and an antisense primer having a base sequence shown in SEQ ID NO: 58 in the sequence listing; a sense primer having a base sequence shown in SEQ ID NO: 59 in the sequence listing, and an antisense primer having a base sequence shown in SEQ ID NO: 60 in the sequence listing; a sense primer having a base sequence shown in SEQ ID NO: 61 in the sequence listing, and an antisense primer having a base sequence shown in SEQ ID NO: 62 in the sequence listing; a sense primer having a base sequence shown in SEQ ID NO: 63 in the sequence listing, and an antisense primer having a base sequence shown in SEQ ID NO: 64 in the sequence listing; a sense primer having a base sequence shown in SEQ ID NO: 65 in the sequence listing, and an antisense primer having a base sequence shown in SEQ ID NO: 66 in the sequence listing; a sense primer having a base sequence shown in SEQ ID NO: 67 in the sequence listing, and an antisense primer having a base sequence shown in SEQ ID NO: 68 in the sequence listing; or a sense primer having a base sequence shown in SEQ ID NO: 69 in the sequence listing, and an antisense primer having a base sequence shown in SEQ ID NO: 70 in the sequence listing.

26. An RT-PCR primer for amplifying a mesenchymal stem cell marker gene which comprises a sense primer having a base sequence shown in SEQ ID NO: 71 in the sequence listing, and an antisense primer having a base sequence shown in SEQ ID NO: 72 in the sequence listing; a sense primer having a base sequence shown in SEQ ID NO: 73 in the sequence listing, and an antisense primer having a base sequence shown in SEQ ID NO: 74 in the sequence listing; a sense primer having a base sequence shown in SEQ ID NO: 75 in the sequence listing, and an antisense primer having a base sequence shown in SEQ ID NO: 76 in the sequence listing; a sense primer having a base sequence shown in SEQ ID NO: 77 in the sequence listing, and an antisense primer having a base sequence shown in SEQ ID NO: 78 in the sequence listing; a sense primer having a base sequence shown in SEQ ID NO: 79 in the sequence listing, and an antisense primer having a base sequence shown in SEQ ID NO: 80 in the sequence listing; a sense primer having a base sequence shown in SEQ ID NO: 81 in the sequence listing, and an antisense primer having a base sequence shown in SEQ ID NO: 82 in the sequence listing; a sense primer having a base sequence shown in SEQ ID NO: 83 in the sequence listing, and an antisense primer having a base sequence shown in SEQ ID NO: 84 in the sequence listing; a sense primer having a base sequence shown in SEQ ID NO: 85 in the sequence listing, and an antisense primer having a base sequence shown in SEQ ID NO: 86 in the sequence listing; a sense primer having a base sequence shown in SEQ ID NO: 87 in the sequence listing, and an antisense primer having a base sequence shown in SEQ ID NO: 88 in the sequence listing; a sense primer having a base sequence shown in SEQ ID NO: 89 in the sequence listing, and an antisense primer having a base sequence shown in SEQ ID NO: 90 in the sequence listing; a sense primer having a base sequence shown in SEQ ID NO: 91 in the sequence listing, and an antisense primer having a base sequence shown in SEQ ID NO: 92 in the sequence listing; a sense primer having a base sequence shown in SEQ ID NO: 93 in the sequence listing, and an antisense primer having a base sequence shown in SEQ ID NO: 94 in the sequence listing; a sense primer having a base sequence shown in SEQ ID NO: 95 in the sequence listing, and an antisense primer having a base sequence shown in SEQ ID NO: 96 in the sequence listing; a sense primer having a base sequence shown in SEQ ID NO: 97 in the sequence listing, and an antisense primer having a base sequence shown in SEQ ID NO: 98 in the sequence listing; a sense primer having a base sequence shown in SEQ ID NO: 99 in the sequence listing, and an antisense primer having a base sequence shown in SEQ ID NO: 100 in the sequence listing; a sense primer having a base sequence shown in SEQ ID NO: 101 in the sequence listing, and an antisense primer having a base sequence shown in SEQ ID NO: 102 in the sequence listing; a sense primer having a base sequence shown in SEQ ID NO: 103 in the sequence listing, and an antisense primer having a base sequence shown in SEQ ID NO: 104 in the sequence listing; a sense primer having a base sequence shown in SEQ ID NO: 105 in the sequence listing, and an antisense primer having a base sequence shown in SEQ ID NO: 106 in the sequence listing; a sense primer having a base sequence shown in SEQ ID NO: 107 in the sequence listing, and an antisense primer having a base sequence shown in SEQ ID NO: 108 in the sequence listing; a sense primer having a base sequence shown in SEQ ID NO: 109 in the sequence listing, and an antisense primer having a base sequence shown in SEQ ID NO: 110 in the sequence listing; a sense primer having a base sequence shown in SEQ ID NO: 111 in the sequence listing, and an antisense primer having a base sequence shown in SEQ ID NO: 112 in the sequence listing; a sense primer having a base sequence shown in SEQ ID NO: 113 in the sequence listing, and an antisense primer having a base sequence shown in SEQ ID NO: 114 in the sequence listing; or a sense primer having a base sequence shown in SEQ ID NO: 115 in the sequence listing, and an antisense primer having a base sequence shown in SEQ ID NO: 116 in the sequence listing.

27. The method for distinguishing a mesenchymal stem cell according to claim 18, wherein a gene in a test cell is amplified by using at least one pair of primers comprising the sense primer and the antisense primer according to any one of claims 24 to 26, and the amplified gene is detected by using the probe for detecting a mesenchymal stem cell marker gene according to claim 10 or 11.

28. A method for distinguishing a mesenchymal stem cell, wherein expression of a mesenchymal stem cell marker polypeptide in a test cell is detected by using the antibody according to any one of claims 15 to 17.

29. A method for distinguishing a mesenchymal stem cell, wherein expression of one or more genes in a group of mesenchymal stem cell marker genes comprising SEQ ID NOs: 1, 11 and 19 in the sequence listing, and expression of one or more genes in a group of mesenchymal stem cell marker genes comprising SEQ ID NOs: 2, 4, 6, 7, 9, 10, 12, 14, 15 and 17 in the sequence listing, in a test cell, are evaluated in combination.

30. A method for distinguishing and separating a mesenchymal stem cell, wherein expression of a marker gene for detecting a mesenchymal stem cell and/or a marker polypeptide for detecting a mesenchymal stem cell in a test cell is detected by using the probe for detecting a mesenchymal stem cell marker gene according to claim 10 or 11 and/or the antibody according to any one of claims 15 to 17, and the distinguished mesenchymal stem cell is separated.

31. The method for distinguishing and separating a mesenchymal stem cell according to claim 30, wherein a mesenchymal stem cell in a test cell is labeled by a fluorescent antibody method using the antibody according to any one of claims 15 to 17, and the labeled mesenchymal stem cell is separated.

32. A kit for distinguishing a mesenchymal stem cell which comprises at least one of the probe for detecting a mesenchymal stem cell marker gene according to claim 10 or 11 the microarray or the DNA chip for detecting a mesenchymal stem cell marker gene according to claim 12 or 13 in which the probe is immobilized, and the antibody according to any one of claims 15 to 17.
